(19) United States
(12) Patent Application Publication
(10) Pub. No.: US 2013/0034813 A1
(43) Pub. Date: Feb. 7, 2013

(54) CHEMICALLY AMPLIFIED POSITIVE RESIST COMPOSITION FOR ArF IMMERSION LITHOGRAPHY AND PATTERN FORMING PROCESS

(71) Applicants: Youichi Ohsawa, Joetsu-shi (JP); Masayoshi Sagehashi, Joetsu-shi (JP); Koji Hasegawa, Joetsu-shi (JP); Takeshi Sasami, Joetsu-shi (JP)

(72) Inventors: Youichi Ohsawa, Joetsu-shi (JP); Masayoshi Sagehashi, Joetsu-shi (JP); Koji Hasegawa, Joetsu-shi (JP); Takeshi Sasami, Joetsu-shi (JP)

(73) Assignee: SHIN-ETSU CHEMICAL CO., LTD., Tokyo (JP)

(21) Appl. No.: 13/558,855

(22) Filed: Jul. 26, 2012

(30) Foreign Application Priority Data
Aug. 5, 2011 (JP) JP2011-171551

(51) Int. Cl. G03F 7/027; C07C 381/12; G03F 7/20; C07C 51/09

(52) U.S. Cl. 430/281.1; 562/586; 430/326

(57) ABSTRACT

A chemically amplified positive resist composition comprising (A) a sulfonium salt of 3,3,3-trifluoro-2-hydroxy-2-trifluoromethylpropionic acid, (B) an acid generator, (C) a base resin, and (D) an organic solvent is suited for ArF immersion lithography. The carboxylic acid sulfonium salt is highly hydrophobic and little leached out in immersion water. By virtue of controlled acid diffusion, a pattern profile with high resolution can be constructed.

### CROSS-REFERENCE TO RELATED APPLICATION

This non-provisional application claims priority under 35 U.S.C. §119(a) on Patent Application No. 2011-171551 filed in Japan on Aug. 5, 2011, the entire contents of which are hereby incorporated by reference.

### TECHNICAL FIELD

This invention relates to (1) a chemically amplified positive resist composition for ArF immersion lithography comprising a specific carboxylic acid sulfonium salt, (2) a pattern forming process using the resist composition, and (3) the synthesis of the carboxylic acid sulfonium salt.

### BACKGROUND ART

While a number of recent efforts are being made to achieve a finer pattern rule in the drive for higher integration and operating speeds in LSI devices, DUV and EUV lithography is thought to hold particular promise as the next generation in microfabrication technology. In particular, photolithography using an ArF excimer laser as the light source is thought requisite to the micropatterning technique capable of achieving a feature size of 0.13 μm or less.

The ArF lithography started partial use from the fabrication of 130-nm node devices and became the main lithography since 90-nm node devices. Although lithography using F₂ laser (157 nm) was initially thought promising as the next lithography for 45-nm node devices, its development was retarded by several problems. A highlight was suddenly placed on the ArF immersion lithography that introduces a liquid having a higher refractive index than air (e.g., water, ethylene glycol, glycerol) between the projection lens and the wafer, allowing the projection lens to be designed to a numerical aperture (NA) of 1.0 or higher and achieving a higher resolution. While the ArF immersion lithography has entered the commercial stage, the technology still needs a resist material which is substantially non-leachable in water.

In the ArF lithography (193 nm), a high sensitivity resist material capable of achieving a high resolution at a small dose of exposure is needed to prevent the degradation of precise and expensive optical system materials. Among several measures for providing high sensitivity resist material, the most common is to select each component which is highly transparent at the wavelength of 193 nm. For example, polyacrylic acid and derivatives thereof, norbornene-maleic anhydride alternating copolymers, polynorbornene, ring-opening metathesis polymerization (ROMP) polymers, and hydrogenated ROMP polymers have been proposed as the base resin. This choice is effective to some extent in enhancing the transparency of a resin alone.

Studies have also been made on photoacid generators (PAGs) and diffusion regulators. Sulfonium salts such as triphenylsulfonium nonalfluorobutanesulfonate are typically used as the PAG because of stability in resist compositions. Amines and weak acid onium salts are typically used as the diffusion regulator. JP-A H11-295887 describes that the addition of triphenylsulfonium acetate ensures to form a satisfactory resist pattern without T-top profile, a difference in line width between isolated and grouped patterns, and standing waves. JP-A H11-327143 reports improvements in sensitivity, resolution and exposure margin by the addition of sulfonic acid ammonium salt or carboxylic acid ammonium salt. Also, JP 4231622 describes that a resist composition for KrF or EB lithography comprising a PAG capable of generating a fluorinated carboxylic acid is improved in resolution and process latitude such as exposure margin and depth of focus. Further, JP 4116340 describes that a resist composition for F₂ laser lithography comprising a PAG capable of generating a fluorinated carboxylic acid is improved in line edge roughness (LER) and solves the footing problem. While these four patent documents refer to the KrF, EB and F₂ lithography, JP 4226803 describes a positive photosensitive composition for ArF excimer laser lithography comprising a carboxylic acid onium salt. These systems are based on the mechanism that a salt exchange occurs between a weak acid onium salt and a strong acid (sulfonic acid) generated by another PAG upon exposure, to form a weak acid and a strong acid onium salt. That is, the strong acid (sulfonic acid) having high acidity is replaced by a weak acid (carboxylic acid), thereby suppressing acid-aided decomposition reaction of acid labile group and reducing or controlling the distance of acid diffusion.

However, even when a weak acid onium salt is used, there still remain problems. Pattern collapse can occur, eventually achieving no improvements in resolution. Low dissolution in alkaline developer may cause defects after development. The salt component can be leached out in immersion liquid (water) to contaminate the immersion lithography tool. The LER problem is not overcome.

### CITATION LIST

Patent Document 1: JP-A H11-295887 (U.S. Pat. No. 6,479,210)
Patent Document 2: JP-A H11-327143
Patent Document 3: JP 4231622 (U.S. Pat. No. 6,485,883)
Patent Document 4: JP 4116340 (U.S. Pat. No. 7,214,467)
Patent Document 5: JP 4226803 (U.S. Pat. No. 6,492,091)

### DISCLOSURE OF INVENTION

An object of the invention is to provide (1) a chemically amplified positive resist composition for ArF immersion lithography comprising a specific carboxylic acid sulfonium salt serving as an acid diffusion control agent so that it may form a resist pattern with improved resolution and minimal defects after development, (2) a pattern forming process using the resist composition, and (3) a method for the synthesis of the carboxylic acid sulfonium salt.

The inventors have found that a resist composition comprising a sulfonium salt of 3,3,3-trifluoro-2-hydroxy-2-trifluoromethylpropionic acid having the general formula (1-1), shown below, as the acid diffusion control agent forms a resist film having improved resolution and minimal defects after development and is suited for high accuracy micropatterning.

In one aspect, the invention provides a chemically amplified positive resist composition for ArF immersion lithography, comprising

(A) a sulfonium salt of 3,3,3-trifluoro-2-hydroxy-2-trifluoromethylpropionic acid having the general formula (1-1):

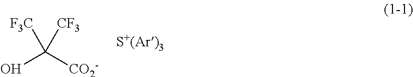

wherein Ar′ is a substituted or unsubstituted C₆-C₂₀ aryl group which may contain a heteroatom, or a plurality of Ar′ groups may bond directly or via an oxygen atom, methylene, sulfone or carbonyl moiety to form an aromatic-containing ring with the sulfur atom to which they are attached,

(B) one or more acid generator having the general formula (1-2):

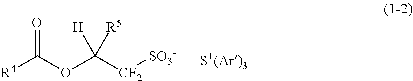

wherein R⁴ is a C₁-C₃₀ alkyl, alkenyl or aralkyl group which may contain a heteroatom, R⁵ is hydrogen or trifluoromethyl, and Ar′ is as defined above,

(C) a base resin having an acidic functional group protected with an acid labile group, which is insoluble or substantially insoluble in alkaline developer, but turns soluble in alkaline developer upon deprotection of the acid labile group, and

(D) an organic solvent.

In another aspect, the invention provides a chemically amplified positive resist composition for ArF immersion lithography, comprising

(A) a sulfonium salt of 3,3,3-trifluoro-2-hydroxy-2-trifluoromethylpropionic acid having the general formula (1-1),

(C′) a base resin having an acidic functional group protected with an acid labile group, which is insoluble or substantially insoluble in alkaline developer, but turns soluble in alkaline developer upon deprotection of the acid labile group, the base resin comprising recurring units having the general formula (1-2′):

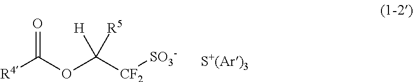

wherein R^{4′} is a backbone portion constituting some recurring units of the base resin, R⁵ is hydrogen or trifluoromethyl, and Ar′ is as defined above, and

(D) an organic solvent.

In a preferred embodiment, the resist composition further comprises a polymer comprising recurring units having the general formula (1a) as a surfactant.

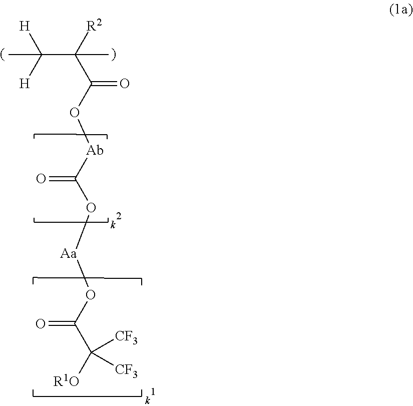

Herein R¹ is hydrogen or a straight, branched or cyclic C₁-C₂₀ monovalent hydrocarbon group in which a constituent moiety —CH₂— may be replaced by —O— or —C(═O)—, R² is hydrogen, fluorine, methyl or trifluoromethyl, Aa is a straight, branched or cyclic C₁-C₂₀ hydrocarbon or fluorinated hydrocarbon group having a valence of k¹+1, Ab is a straight, branched or cyclic C₁-C₆ divalent hydrocarbon group, k¹ is an integer of 1 to 3, and k² is 0 or 1.

In a preferred embodiment, the base resin comprises recurring units having an acid labile group represented by the general formula (3) and recurring units of at least one type selected from the general formulae (4) to (6).

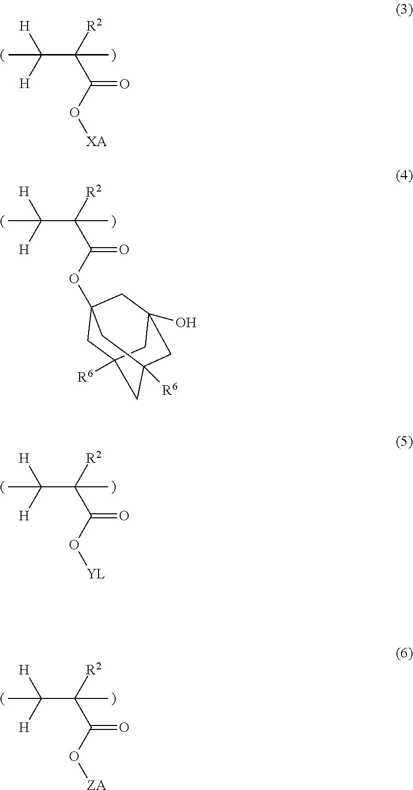

Herein R² is hydrogen, fluorine, methyl or trifluoromethyl, XA is an acid labile group, R⁶ is each independently hydrogen or hydroxyl, YL is a substituent group having a lactone structure, ZA is hydrogen, a C₁-C₁₅ fluoroalkyl group or C₁-C₁₅ fluoroalcohol-containing substituent group.

In a further aspect, the invention provides a pattern forming process comprising the steps of applying the resist composition defined above onto a substrate, prebaking to form a resist film, exposing the resist film to high-energy radiation through a photomask while interposing water between the substrate and a projection lens, optionally baking, and developing in a developer.

In a still further aspect, the invention provides a method for synthesizing a sulfonium salt of 3,3,3-trifluoro-2-hydroxy-2-trifluoromethylpropionic acid having the general formula (1-1), comprising the steps of providing methyl 3,3,3-trifluoro-2-hydroxy-2-trifluoromethylpropionate, effecting hydrolysis reaction into 3,3,3-trifluoro-2-hydroxy-2-trifluoromethylpropionic acid or a salt thereof, and obtaining the desired sulfonium salt therefrom.

In a yet further aspect, the invention provides a resist composition comprising a sulfonium salt of 3,3,3-trifluoro-2-hydroxy-2-trifluoromethylpropionic acid having the general formula (1-1) as synthesized by the above method.

It is noted that the resist composition of the invention can be applied to the immersion lithography. The immersion lithography is designed to expose the prebaked resist film to light from a projection lens with an immersion medium interposed between the resist film and the projection lens. The ArF immersion lithography generally uses pure water as the immersion medium. This technology, combined with a projection lens having a NA of at least 1.0, is important for the ArF lithography to survive to the 65 nm node and forth, with a further development thereof being accelerated.

The resist composition of the invention allows the feature size of the pattern after development to be reduced by various shrinkage techniques. For example, the hole size can be shrunk by such known techniques as thermal flow, RELACS, SAFIRE, and WASOOM. More effective shrinkage of hole size by thermal flow is possible particularly when a hydrogenated cycloolefin ROMP polymer having a low Tg or the like is blended in the composition.

### ADVANTAGEOUS EFFECTS OF INVENTION

When used in resist material, the specific carboxylic acid sulfonium salt is highly hydrophobic because of a fluorinated anion. The salt has advantages of least leaching in immersion water and controlled acid diffusion, and enables to construct a pattern profile with high resolution. Since the specific carboxylic acid sulfonium salt which has not undergone decomposition or acid exchange by exposure has high solubility in or high affinity to alkaline developer, few defects form after development. Thus the chemically amplified positive resist composition is quite useful for the ArF immersion lithography.

### DESCRIPTION OF EMBODIMENTS

As used herein, the singular forms “a,” “an” and “the” include plural referents unless the context clearly dictates otherwise. The notation (Cn-Cm) means a group containing from n to m carbon atoms per group.

The abbreviations and acronyms have the following meaning.

Mw: weight average molecular weight

Mn: number average molecular weight

Mw/Mn: molecular weight distribution or dispersity

GPC: gel permeation chromatography

PEB: post-exposure bake

PAG: photoacid generator

In structural formulae, the broken line indicates a valence bond. Me, Ph, and Ac stand for methyl, phenyl, and acetyl, respectively.

A first embodiment of the invention is a chemically amplified positive resist composition for ArF immersion lithography, comprising as essential components,

(A) a sulfonium salt of 3,3,3-trifluoro-2-hydroxy-2-trifluoromethylpropionic acid having the general formula (1-1),

(B) at least one acid generator having the general formula (1-2),

(C) a base resin having an acidic functional group protected with an acid labile group, which is insoluble or substantially insoluble in alkaline developer, but turns soluble in alkaline developer upon deprotection of the acid labile group, and

(D) an organic solvent.

The sulfonium salt having formula (1-1) is described in detail.

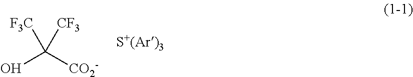

Herein Ar′ is a substituted or unsubstituted C₆-C₂₀ aryl group which may contain a heteroatom, or a plurality of Ar′ groups may bond directly or via an oxygen atom, methylene, sulfone or carbonyl moiety to form an aromatic-containing ring with the sulfur atom to which they are attached.

In formula (1-1), Ar′ is a substituted or unsubstituted C₆-C₂₀ aryl group which may contain a heteroatom, or a plurality of Ar′ groups may bond directly or via an oxygen atom, methylene, sulfone or carbonyl moiety to form a ring. Typical of the heteroatom contained are oxygen, nitrogen, sulfur and halogen atoms, with the oxygen or fluorine atom being preferred. Suitable substituent radicals include straight, branched or cyclic C₁-C₆ alkyl radicals, straight, branched or cyclic C₁-C₆ alkoxy radicals, alkoxyalkyl radicals, hydroxyl radicals, fluoro, chloro, N,N-dialkylamino radicals in which the alkyl moiety has 1 to 4 carbon atoms, mono- or polycyclic lactone radicals of 4 to 10 carbon atoms, straight, branched or cyclic C₃-C₁₄ alkyloxycarbonylmethoxy radicals, methylthio radicals, phenylthio radicals, and C₁-C₁₁ acyloxy radicals. Although the number of substituent radicals is arbitrary, mono or di-substitution is preferred, if any, with mono-substitution being most preferred. Exemplary substituent radicals include

methyl, ethyl, propyl, 1-methylethyl, butyl, 1,1-dimethylethyl, hexyl, cyclohexyl, methoxy, ethoxy, propoxy, butoxy, 1,1-dimethylethoxy, hexyloxy, cyclohexyloxy, 2-methoxyethoxy, 2-(2-methoxyethoxy)ethoxy, 2,2,2-trifluoroethoxy, N,N-dimethylamino, 1,1-dimethylethoxycarbonylmethoxy, 1-methyladamantan-1-yloxycarbonylmethoxy, acetyl, pivaloyloxy, and adamantan-1-ylcarbonyloxy.

Suitable groups of Ar′ include, but are not limited to, phenyl, naphthyl (with any substitution position to the sulfur atom of sulfonium cation), anthryl, phenanthryl, pyrenyl, tolyl, xylyl, trimethylphenyl (with any substitution position to the sulfur atom of sulfonium cation), ethylphenyl, biphenylyl, methoxyphenyl, fluorophenyl, difluorophenyl, t-butylphenyl, ethoxyphenyl, butoxyphenyl, t-butoxyphenyl, methylthiophenyl, trifluoromethylphenyl, acetoxyphenyl, hydroxyphenyl, N,N-dimethylaminophenyl, methylnaphthyl, hydroxynaphthyl, dihydroxynaphthyl, methoxynaphthyl, butoxynaphthyl, 2,2,2-trifluoroethoxynaphthyl, and (2-methoxyethoxy)naphthyl. Also included are aryl groups having a polymerizable substituent radical such as acryloyloxy or methacryloyloxy. Exemplary such groups include 4-acryloyloxyphenyl, 4-methacryloyloxyphenyl, 4-acryloyloxy-3,5-dimethylphenyl, 4-methacryloyloxy-3,5-dimethylphenyl, 4-vinyloxyphenyl, and 4-vinylphenyl. Inter alia, phenyl, 4-tert-butylphenyl, and 4-tert-butoxyphenyl groups are preferred.

When a plurality of Ar′ groups bond directly or via an oxygen atom, methylene, sulfone or carbonyl moiety, dibenzothiophene skeleton, phenoxathiin skeleton, and other partial structures as shown below are exemplary.

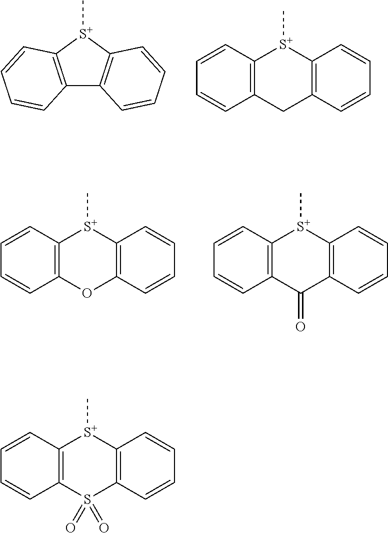

Herein the broken line denotes a bond to another Ar′ group.

Examples of the sulfonium cation include, but are not limited to, triphenylsulfonium,

4-hydroxyphenyldiphenylsulfonium, bis(4-hydroxyphenyl)phenylsulfonium, tris(4-hydroxyphenyl)sulfonium, 4-tert-butoxyphenyldiphenylsulfonium, bis(4-tert-butoxyphenyl)phenylsulfonium, tris(4-tert-butoxyphenyl)sulfonium, 3-tert-butoxyphenyldiphenylsulfonium, bis(3-tert-butoxyphenyl)phenylsulfonium, tris(3-tert-butoxyphenyl)sulfonium, 4-tert-butylphenyldiphenylsulfonium, tris(4-tert-butylphenyl)sulfonium, 3,4-di-tert-butoxyphenyldiphenylsulfonium, bis(3,4-di-tert-butoxyphenyl)phenylsulfonium, tris(3,4-di-tert-butoxyphenyl)sulfonium, diphenyl(4-thiophenoxyphenyl)sulfonium, 10-phenylphenoxathiinium, S-phenyldibenzothiophenium, 4-tert-butoxycarbonylmethyloxyphenyldiphenylsulfonium, tris(4-tert-butoxycarbonylmethyloxyphenyl)sulfonium, (4-tert-butoxyphenyl)bis(4-dimethylaminophenyl)sulfonium, tris(4-dimethylaminophenyl)sulfonium, 2-naphthyldiphenylsulfonium, (4-hydroxy-3,5-dimethylphenyl)diphenylsulfonium, and (4-n-hexyloxy-3,5-dimethylphenyl)diphenylsulfonium. Also included are 4-methacryloyloxyphenyldiphenylsulfonium, 4-acryloyloxyphenyldiphenylsulfonium, 4-methacryloyloxyphenyldimethylsulfonium, 4-acryloyloxyphenyldimethylsulfonium, (4-methacryloyloxy-3,5-dimethylphenyl)diphenylsulfonium, and (4-acryloyloxy-3,5-dimethylphenyl)diphenylsulfonium. Of these, triphenylsulfonium, 4-tert-butylphenyldiphenylsulfonium, 4-tert-butoxyphenyldiphenylsulfonium, 10-phenylphenoxathiinium, and S-phenyldibenzothiophenium are more preferred. Inter alia, triphenylsulfonium, 4-tert-butylphenyldiphenylsulfonium, and 4-tert-butoxyphenyldiphenylsulfonium are most preferred.

The sulfonium cation may be a so-called alkylsulfonium cation that is a cation having an alkyl group directly bonded thereto. However, since a combination of an alkylsulfonium cation which is highly active to high-energy radiation and a nucleophilic agent (such as 4-butoxynaphthyl-1-thiacyclo-pentanium cation) with 3,3,3-trifluoro-2-hydroxy-2-trifluoro-methylpropionic acid anion often has low stability by itself or in resist solution, a so-called triarylsulfonium cation as represented by formula (1-1) is desirable in order to provide such a combination with stability.

The specific carboxylic acid used herein is per se known and may be synthesized by addition of hydrogen cyanide to hexafluoroacetone and subsequent hydrolysis. However, hexafluoroacetone and hydrogen cyanide are deadly toxic and need careful handling.

Another embodiment of the invention is a method for synthesizing a sulfonium salt of 3,3,3-trifluoro-2-hydroxy-2-trifluoromethylpropionic acid having the general formula (1-1) by hydrolysis reaction of methyl 3,3,3-trifluoro-2-hydroxy-2-trifluoromethylpropionate into a corresponding carboxylic acid or a salt thereof, and converting it into the desired sulfonium salt.

Notably, 3,3,3-trifluoro-2-hydroxy-2-(trifluoromethyl)-propionic acid derivatives, especially methyl esters thereof, are obtainable by starting with octafluoroisobutylene which is one of byproducts formed during synthesis of hexafluoropropene, for example. Since the starting reactant is available as a byproduct of a commercially useful product, it is a fluorine compound supplied in plenty and at relatively low cost.

Specifically, the hydrolysis reaction is preferably base hydrolysis. Suitable bases include lithium hydroxide, sodium hydroxide, potassium hydroxide, calcium hydroxide, tetramethylammonium hydroxide, tetraethylammonium hydroxide, and tetrabutylammonium hydroxide, with sodium hydroxide and tetramethylammonium hydroxide being preferred. It is also possible to synthesize the desired sulfonium salt having formula (1-1) directly using triphenylsulfonium hydroxide.

An amount of the base is 1 to 3 equivalents, preferably 1 to 1.2 equivalents. The reaction medium may be water, water/methanol, water/ethanol or the like, with water being preferred. The reaction temperature and time are arbitrary. It is preferred to heat at 60 to 80° C. to accelerate consumption of the starting reactant. The reaction solution may be used directly as the carboxylic acid salt solution in the subsequent step of ion exchange reaction. Alternatively, the reaction solution may be concentrated, from which the carboxylic acid salt may be recovered in crude crystal form. An acid such as hydrochloric acid may be added to neutralize the basicity, or distillation/purification may be effected in the co-presence of sulfuric acid, thereby recovering the carboxylic acid.

Suitable salts of 3,3,3-trifluoro-2-hydroxy-2-(trifluoromethyl)propionic acid include lithium, sodium, potassium, calcium, tetramethylammonium, tetraethylammonium, and tetrabutylammonium salts.

The synthesis of sulfonium cation is well known. Synthesis may be carried out according to the teachings of JP-A 2007-145797, JP-A 2009-007327, and JP-A 2009-091350, for example.

With respect to the polymerizable sulfonium cation, reference may be made to JP-A H04-230645 and JP-A 2005-84365. The polymerizable sulfonium cation may also be used as the monomer from which units of a polymer to be described later are derived.

Ion exchange reaction between the foregoing 3,3,3-trifluoro-2-hydroxy-2-(trifluoromethyl)propionic acid or a salt thereof and a sulfonium salt may be performed in an organic solvent (e.g., dichloromethane, ethyl acetate, methyl isobutyl ketone, methanol, ethanol or acetonitrile) alone or in admixture with water. After removal of the salt component by-produced, the product may be purified by a standard technique such as recrystallization or chromatography.

The specific carboxylic acid sulfonium salt having formula (1-1) may be used alone or in admixture of two or more.

In the resist composition, the specific carboxylic acid sulfonium salt having formula (1-1) may be used in any desired amount as long as the benefits of the invention are not compromised. An appropriate amount is 0.1 to 10 parts, more preferably 0.1 to 8 parts by weight per 100 parts by weight of the base resin to be described later. Outside the range, an excess amount of the salt having formula (1-1) may cause a lowering of sensitivity and degradation of resolution.

Component (B) is a photoacid generator having the general formula (1-2):

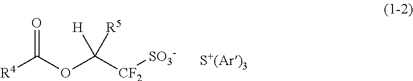

wherein R⁴ is a C₁-C₃₀ alkyl, alkenyl or aralkyl group which may contain a heteroatom, R⁵ is hydrogen or trifluoromethyl, and Ar′ is as defined above.

In formula (1-2), R⁴ is a C₁-C₃₀ alkyl, alkenyl or aralkyl group which may contain a heteroatom. Exemplary heteroatoms which may be contained in R⁴ include oxygen, nitrogen, sulfur and halogen atoms, with oxygen being preferred. The C₁-C₃₀ alkyl, alkenyl or aralkyl group may be straight, branched or cyclic while it is preferred for forming a fine feature size pattern of high resolution that these groups have 6 to 30 carbon atoms. It is undesirable that R⁴ be aryl, because the resulting resist pattern may have less smooth sidewalls. Suitable groups of R⁴ include, but are not limited to, methyl, ethyl, propyl, isopropyl, butyl, isobutyl, s-butyl, t-butyl, pentyl, neopentyl, cyclopentyl, hexyl, cyclohexyl, 3-cyclohexenyl, heptyl, 2-ethylhexyl, nonyl, undecyl, tridecyl, pentadecyl, heptadecyl, 1-adamantyl, 2-adamantyl, 1-adamantylmethyl, norbornyl, norbornylmethyl, tricyclodecanyl, tetracyclododecanyl, tetracyclododecanylmethyl, dicyclohexylmethyl, eicosanyl, allyl, benzyl, diphenylmethyl, tetrahydrofuryl, methoxymethyl, ethoxymethyl, methylthiomethyl, acetamidomethyl, trifluoromethyl, (2-methoxyethoxy)methyl, acetoxymethyl, 2-carboxy-1-cyclohexyl, 2-oxopropyl, 4-oxo-1-adamantyl, and 3-oxocyclohexyl. When R⁴ stands for a backbone of the base resin (C), preferably the acid generator is incorporated within a base resin by copolymerizing a monomer wherein R⁴ is vinyl or isopropenyl during preparation of the base resin.

In formula (1-2), R⁵ is hydrogen or trifluoromethyl. It is preferred that R⁵ be trifluoromethyl, because the corresponding acid generator having formula (1-2) is more soluble in solvent.

With respect to Ar′ in formula (1-2), the same as described in conjunction with formula (1-1) is true.

With respect to the synthesis of sulfonium salt having formula (1-2), reference may be made to JP-A 2007-145797, JP-A 2008-106045, JP-A 2009-007327, and JP-A 2009-258695.

Preferred examples of the PAG are given below.

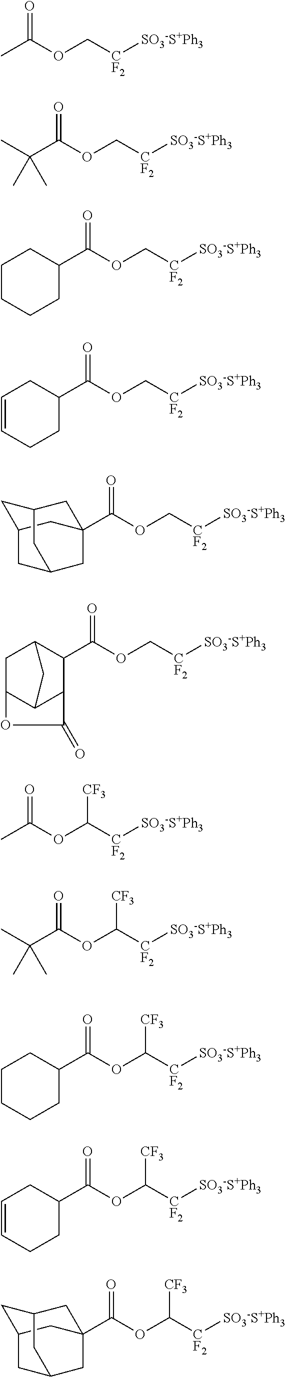

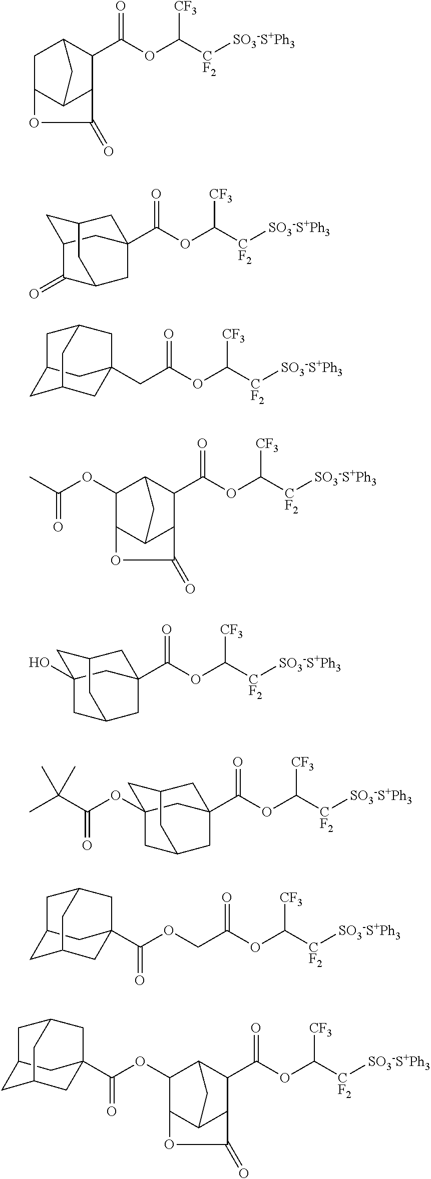

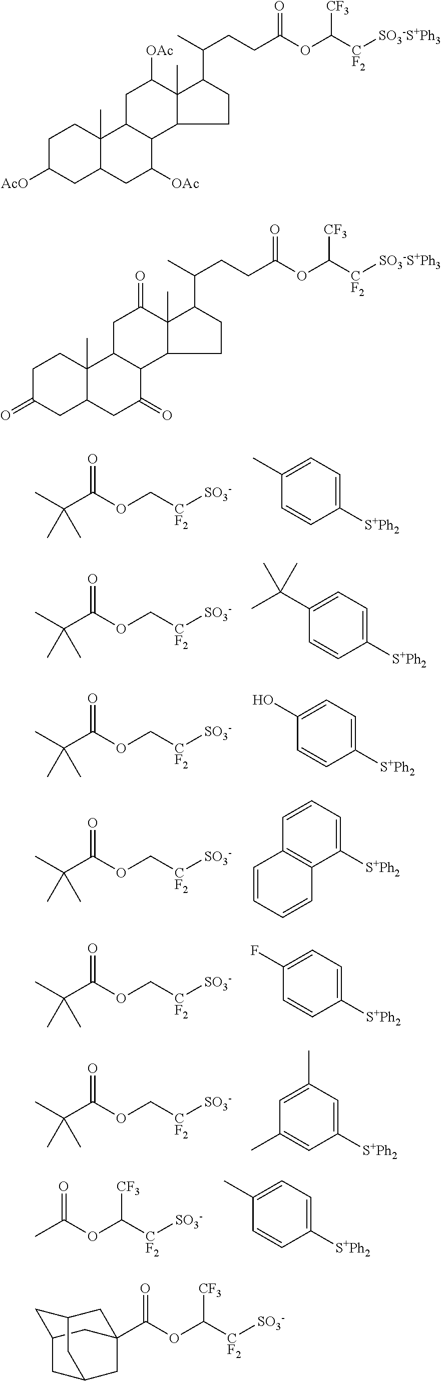

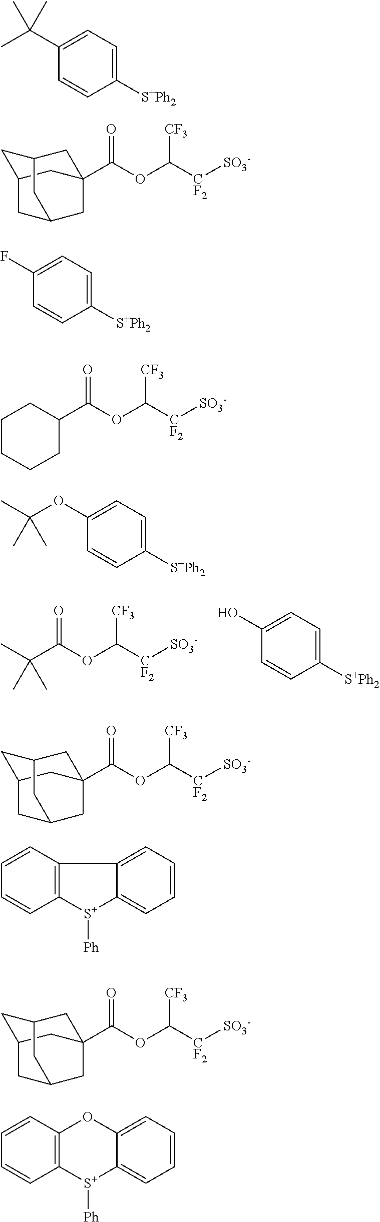

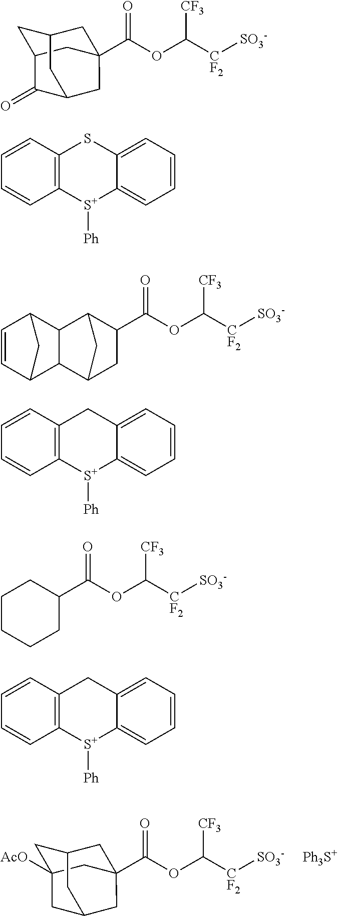

In the acid generator having formula (1-2) compounded in the resist composition, difluoromethylene is at α-position of the sulfonate group in the anion. Therefore, the acid generated by this acid generator is a super-strong acid equivalent to trifluoromethanesulfonic acid or the like, so that decomposition reaction of the resist base resin in the exposed region may proceed to a full extent, affording a high dissolution contrast. In addition, the presence of acyloxy group in the anion increases polarity and molecular weight, controls the volatility and diffusion rate of the generated acid, and eventually contributes to an improvement in resolution of a fine pattern. By a choice of R⁴, R⁵ and Ar′ in formula (1-2) from their alternatives, the properties (including transmittance, acid generation efficiency, solvent solubility, polarity, hydrophilicity, in-film distribution, and stability) of the acid generator and the properties (including acidity, diffusion rate, volatility, and affinity to base resin) of the generated acid can be adjusted in accordance with a particular resist base resin and exposure method used. Accordingly the performance (including resolution) of the resist composition can be adjusted optimum.

An appropriate amount of the acid generator having formula (1-2) used is 0.1 to 40 parts, more preferably 1 to 20 parts by weight per 100 parts by weight of the base resin (C). Less than 0.1 part of the acid generator may generate a less amount of necessary acid upon exposure, leading to low sensitivity and resolution. More than 40 parts of the acid generator may interfere with formation of a satisfactory resist film and reduce the transmittance and resolution thereof.

In addition to the PAG of formula (1-2), another PAG may be added. The other PAG may be any compound capable of generating an acid upon exposure to high-energy radiation including UV, DUV, EB, EUV, x-ray, excimer laser, γ-ray, and synchrotron radiation. Suitable PAGs include sulfonium salts, iodonium salts, sulfonyldiazomethane, N-sulfonyloxydicarboxylmide, O-arylsulfonyloxime, and O-alkylsulfonyloxime generators. The other acid generators may be used alone or in admixture of two or more.

Sulfonium salts are salts of sulfonium cations with sulfonates, bis(substituted alkylsulfonyl)imides and tris(substituted alkylsulfonyl)methides. Exemplary sulfonium cations include those sulfonium cations described in conjunction with formula (1-2). Exemplary sulfonates include trifluoromethanesulfonate, pentafluoroethanesulfonate, heptafluoropropanesulfonate, nonafluorobutanesulfonate, tridecafluorohexanesulfonate, 2,2,2-trifluoroethanesulfonate, pentafluorobenzenesulfonate, 1,1-difluoro-2-naphthylethanesulfonate, 1,1,2,2-tetrafluoro-2-(norbornan-2-yl)ethanesulfonate, 1,1,2,2-tetrafluoro-2-(tetracyclo[6.2.1.1^{3,6}.0^{2,7}]dodec-3-en-8-yl)ethanesulfonate, 2-benzoyloxy-1,1,3,3,3-pentafluoropropanesulfonate, 1,1-difluoro-2-tosyloxyethanesulfonate, adamantanemethoxycarbonyldifluoromethanesulfonate, 1-(3-hydroxymethyladamantane)methoxycarbonyldifluoromethane-sulfonate, methoxycarbonyldifluoromethanesulfonate, 1-(hexahydro-2-oxo-3,5-methano-2H-cyclopenta[b]furan-6-yl-oxycarbonyl)difluoromethanesulfonate, and 4-oxo-1-adamantyloxycarbonyldifluoromethanesulfonate. Exemplary bis(substituted alkylsulfonyl)imides include bis(trifluoromethylsulfonyl)imide, bis(pentafluoroethylsulfonyl)imide, bis(heptafluoropropylsulfonyl)imide, and perfluoro(1,3-propylenebissulfonyl)imide. A typical tris(substituted alkylsulfonyl)methide is tris(trifluoromethylsulfonyl)methide. Sulfonium salts based on combination of the foregoing examples are included.

Examples of the iodonium salt, N-sulfonyloxydicarboxylmide, O-arylsulfonyloxime, and O-alkylsulfonyloxime acid generators are described in JP-A 2009-269953.

Preferred examples of the other PAG include triphenylsulfonium nonafluorobutanesulfonate, triphenylsulfonium bis(trifluoromethylsulfonyl)imide, triphenylsulfonium perfluoro(1,3-propylenebissulfonyl)imide, triphenylsulfonium tris(trifluoromethanesulfonyl)methide, N-nonafluorobutanesulfonyloxy-1,8-naphthalenedicarboxylmide, 2-(2,2,3,3,4,4-hexafluoro-1-(nonafluorobutylsulfonyloxy-imino)butyl)fluorene, and 2-(2,2,3,3,4,4,5,5-octafluoro-1-(nonafluorobutylsulfonyloxy-imino)pentyl)fluorene.

The amount of the other PAG added is not particularly limited as long as the benefits of the invention are not compromised. An appropriate amount of the other PAG added is 0 to 20 parts, more preferably 0.1 to 10 parts by weight per 100 parts by weight of the base resin. Too high a proportion of the other PAG may give rise to problems such as degraded resolution and foreign particles during development and resist film stripping. The other PAG may be used alone or in admixture of two or more. A total amount of the acid generator having formula (1-2) and the other acid generator is preferably 0.1 to 40 parts, more preferably 1 to 20 parts by weight per 100 parts by weight of the base resin.

The acid generators having formula (1-2) may be used alone or in admixture of two or more. It is possible to control the transmittance of a resist film by selecting an acid generator having a low transmittance at the exposure wavelength and adjusting the amount of the acid generator added. If desired, the acid generator having formula (1-2) may be used in combination with another known acid generator. The other known acid generator which can be used in combination is not particularly limited and may be selected from the compounds described in U.S. Pat. No. 7,537,880 (JP-A 2008-111103, paragraphs [0122] to [0142]).

Component (C) is a base resin having an acidic functional group protected with an acid labile group, which resin is insoluble or substantially insoluble in alkaline developer, but turns soluble in alkaline developer upon deprotection of the acid labile group.

In the other embodiment, the base resin is (C′) a base resin having an acidic functional group protected with an acid labile group, wherein the base resin is insoluble or substantially insoluble in alkaline developer, but turns soluble in alkaline developer upon deprotection of the acid labile group, and the base resin comprises recurring units having the general formula (1-2′):

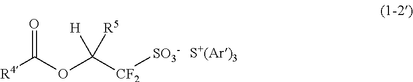

wherein R^{4′} is a backbone portion constituting some recurring units of the base resin, R⁵ is hydrogen or trifluoromethyl, and Ar′ is as defined above.

In the other embodiment using the base resin comprising recurring units having formula (1-2′), the acid generator (B) need not be compounded. In formula (1-2′), R^{4′} may be either of the following.

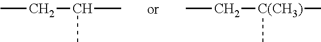

It is noted that the broken line denotes a valence bond to the carbon atom of the carbonyl group (C═O).

When the acid generator has formula (1-2) wherein R⁴ is R^{4′}, that is, represents a backbone of the base resin (C), it is meant that the recurring unit corresponding to formula (1-2) is contained in the base resin. In this case, the acid generated by the acid generator is bound to the polymer, so that acid diffusion may be fully restrained. This is desirable for the purpose of forming a fine pattern having a pitch of less than 80 nm. Examples of the recurring unit in this embodiment are given below, but not limited thereto.

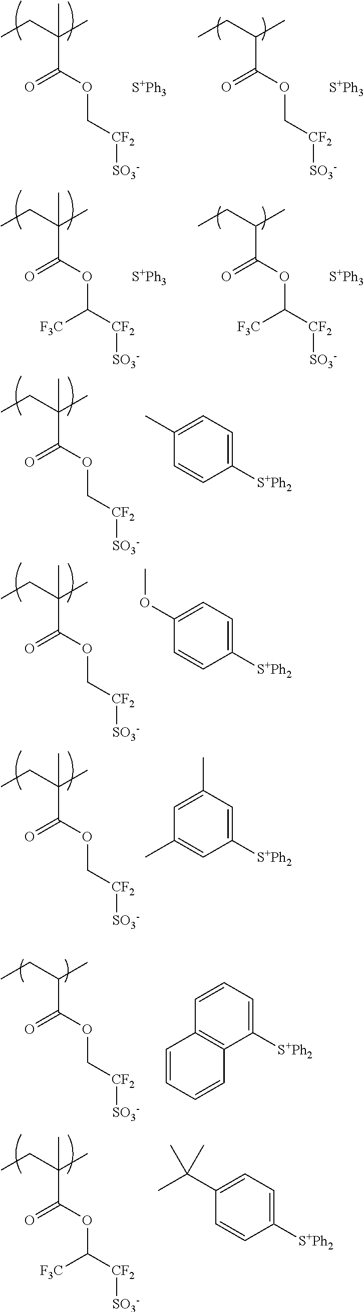

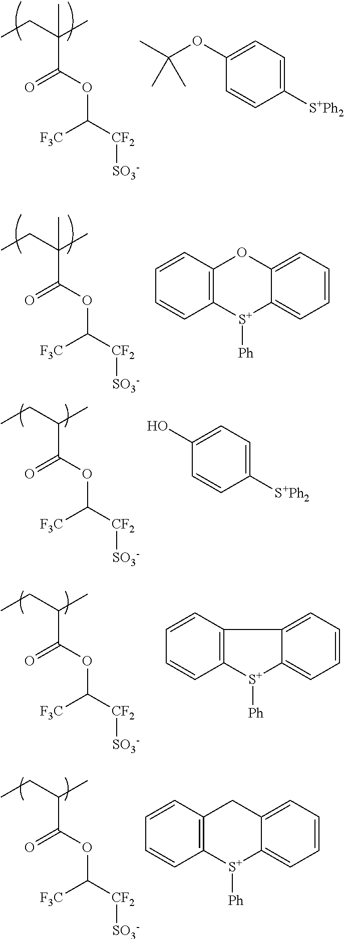

In the embodiment wherein recurring units having formula (1-2′) are incorporated in the base resin, the content of recurring units having formula (1-2′) is preferably 0.2 to 20 mol %, more preferably 0.5 to 15 mol % based on the total recurring units of the base resin. Too low a content may fail to achieve the incorporation effect whereas too high a content may reduce the solvent solubility of a base resin, causing more coating defects.

With respect to the base resin comprising recurring units having formula (1-2′), reference may be made to U.S. Pat. Nos. 7,569,326 and 8062828 (JP-A 2008-133448 and 2009-217253).

Examples of the base resin or polymer (C) include, but are not limited to, (meth)acrylic acid ester polymers, alternating copolymers of cycloolefin with maleic anhydride, copolymers further containing vinyl ethers or (meth)acrylic acid esters, polynorbornene, cycloolefin ring-opening metathesis polymerization (ROMP) polymers, and hydrogenated cycloolefin ROMP polymers. The base resins may be used alone or in admixture of two or more. In the case of positive resist compositions, it is a common practice to substitute an acid labile group for the hydroxyl moiety of a carboxyl group for reducing the rate of dissolution in unexposed regions.

In a preferred embodiment, the base resin may comprise recurring units having an acid labile group represented by the general formula (3) and recurring units of at least one type selected from the general formulae (4) to (6).

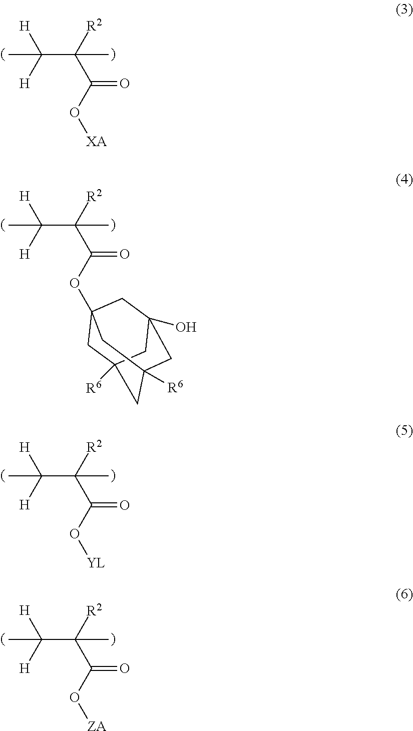

Herein R² is hydrogen, fluorine, methyl or trifluoromethyl. XA is an acid labile group. R⁶ is each independently hydrogen or hydroxyl. YL is a substituent group having a lactone structure. ZA is hydrogen, a C₁-C₁₅ fluoroalkyl group or C₁-C₁₅ fluoroalcohol-containing substituent group.

A polymer comprising recurring units having formula (3) functions such that it may be decomposed under the action of an acid and turn alkali soluble. The acid labile group represented by XA may be selected from a variety of such groups. Examples of the acid labile group include groups of the following general formulae (L1) to (L4), tertiary alkyl groups of 4 to 20 carbon atoms, preferably 4 to 15 carbon atoms, trialkylsilyl groups in which each alkyl moiety has 1 to 6 carbon atoms, and oxoalkyl groups of 4 to 20 carbon atoms.

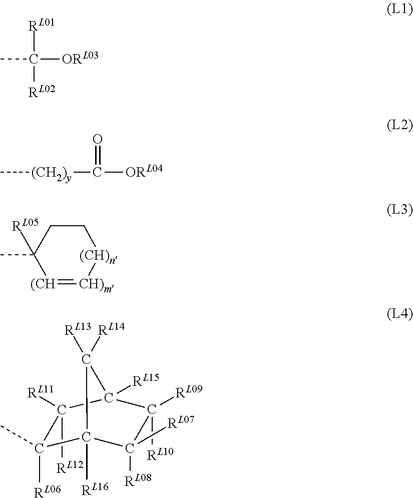

It is noted that the broken line denotes a valence bond.

In formula (L1), R^{L01} and R^{L02} are hydrogen or straight, branched or cyclic alkyl groups of 1 to 18 carbon atoms, preferably 1 to 10 carbon atoms. Exemplary alkyl groups include methyl, ethyl, propyl, isopropyl, n-butyl, sec-butyl, tert-butyl, cyclopentyl, cyclohexyl, 2-ethylhexyl, n-octyl, norbornyl, tricyclodecanyl, tetracyclododecanyl, and adamantyl. R^{L03} is a monovalent hydrocarbon group of 1 to 18 carbon atoms, preferably 1 to 10 carbon atoms, which may contain a hetero atom such as oxygen, examples of which include unsubstituted straight, branched or cyclic alkyl groups and straight, branched or cyclic alkyl groups in which some hydrogen atoms are replaced by hydroxyl, alkoxy, oxo, amino, alkylamino or the like. Exemplary straight, branched or cyclic alkyl groups include methyl, ethyl, propyl, isopropyl, n-butyl, sec-butyl, tert-butyl, cyclopentyl, cyclohexyl, 2-ethylhexyl, n-octyl, norbornyl, tricyclodecanyl, tetracyclododecanyl, and adamantyl. Illustrative examples of the substituted alkyl groups are shown below.

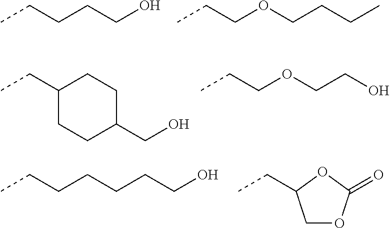

A pair of R^{L01} and R^{L02}, R^{L01} and R^{L03}, or R^{L02} and R^{L03} may bond together to form a ring with the carbon and oxygen atom to which they are attached. Each of R^{L01}, R^{L02} and R^{L03} is a straight or branched alkylene group of 1 to 18 carbon atoms, preferably 1 to 10 carbon atoms when they form a ring.

In formula (L2), R^{L04} is a tertiary alkyl group of 4 to 20 carbon atoms, preferably 4 to 15 carbon atoms, a trialkylsilyl group in which each alkyl moiety has 1 to 6 carbon atoms, an oxoalkyl group of 4 to 20 carbon atoms, or a group of formula (L1). Exemplary tertiary alkyl groups are tert-butyl, tert-amyl, 1,1-diethylpropyl, 2-cyclopentylpropan-2-yl, 2-cyclohexylpropan-2-yl, 2-(bicyclo[2.2.1]heptan-2-yl)propan-2-yl, 2-(adamantan-1-yl)propan-2-yl, 1-ethylcyclopentyl, 1-butylcyclopentyl, 1-ethylcyclohexyl, 1-butylcyclohexyl, 1-ethyl-2-cyclopentenyl, 1-ethyl-2-cyclohexenyl, 2-methyl-2-adamantyl, and 2-ethyl-2-adamantyl. Exemplary trialkylsilyl groups are trimethylsilyl, triethylsilyl, and dimethyl-tert-butylsilyl. Exemplary oxoalkyl groups are 3-oxocyclohexyl, 4-methyl-2-oxooxan-4-yl, and 5-methyl-2-oxooxolan-5-yl. Letter y is an integer of 0 to 6.

In formula (L3), R^{L05} is an optionally substituted, straight, branched or cyclic C₁-C₈ alkyl group or an optionally substituted C₆-C₂₀ aryl group. Examples of the optionally substituted alkyl group include straight, branched or cyclic alkyl groups such as methyl, ethyl, propyl, isopropyl, n-butyl, sec-butyl, tert-butyl, tert-amyl, n-pentyl, n-hexyl, cyclopentyl and cyclohexyl, and substituted forms of the foregoing in which some hydrogen atoms are substituted by hydroxyl, alkoxy, carboxyl, alkoxycarbonyl, oxo, amino, alkylamino, cyano, mercapto, alkylthio, sulfo or the like. Examples of the optionally substituted aryl groups include phenyl, methylphenyl, naphthyl, anthryl, phenanthryl, and pyrenyl. Letter m′ is equal to 0 or 1, n′ is equal to 0, 1, 2 or 3, and 2m′+n′ is equal to 2 or 3.

In formula (L4), R^{L06} is an optionally substituted, straight, branched or cyclic C₁-C₈ alkyl group or an optionally substituted C₆-C₂₀ aryl group. Examples of these groups are the same as exemplified for R^{L05}. R^{L07} to R^{L16} independently represent hydrogen or C₁-C₁₅ monovalent hydrocarbon groups. Exemplary hydrocarbon groups are straight, branched or cyclic alkyl groups such as methyl, ethyl, propyl, isopropyl, n-butyl, sec-butyl, tert-butyl, tert-amyl, n-pentyl, n-hexyl, n-octyl, n-nonyl, n-decyl, cyclopentyl, cyclohexyl, cyclopentylmethyl, cyclopentylethyl, cyclopentylbutyl, cyclohexylmethyl, cyclohexylethyl and cyclohexylbutyl, and substituted forms of the foregoing in which some hydrogen atoms are replaced by hydroxyl, alkoxy, carboxyl, alkoxycarbonyl, oxo, amino, alkylamino, cyano, mercapto, alkylthio, sulfo or other groups. Alternatively, two of R^{L07} to R^{L16} may bond together to form a ring with the carbon atom to which they are attached (for example, a pair of R^{L07} and R^{L08}, R^{L07} and R^{L09}, R^{L08} and R^{L10}, R^{L09} and R^{L10}, R^{L11} and R^{L12}, R^{L13} and R^{L14}, or a similar pair form a ring).

Each of R^{L07} to R^{L16} represents a divalent C₁-C₁₅ hydrocarbon group when they form a ring, examples of which are the ones exemplified above for the monovalent hydrocarbon groups, with one hydrogen atom being eliminated. Two of R^{L07} to R^{L16} which are attached to vicinal carbon atoms may bond together directly to form a double bond (for example, a pair of R^{L07} and R^{L09}, R^{L09} and R^{L15}, R^{L13} and R^{L15}, or a similar pair).

Of the acid labile groups of formula (L1), the straight and branched ones are exemplified by the following groups.

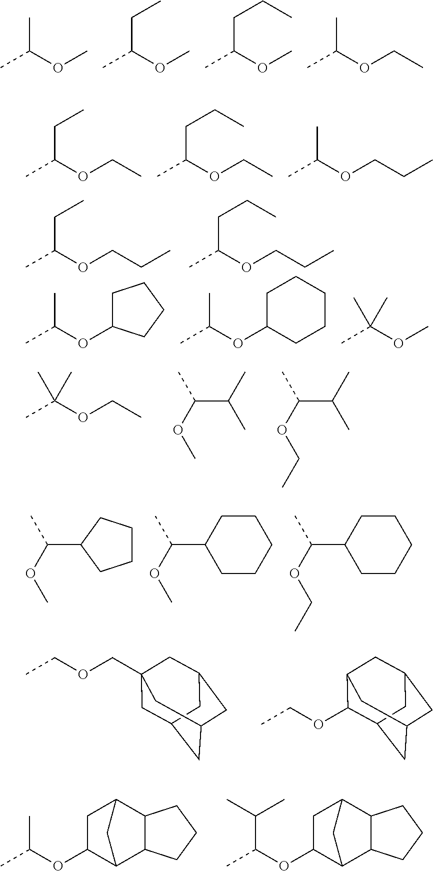

Of the acid labile groups of formula (L1), the cyclic ones are, for example, tetrahydrofuran-2-yl, 2-methyltetrahydrofuran-2-yl, tetrahydropyran-2-yl, and 2-methyltetrahydropyran-2-yl.

Examples of the acid labile groups of formula (L2) include tert-butoxycarbonyl, tert-butoxycarbonylmethyl, tert-amyloxycarbonyl, tert-amyloxycarbonylmethyl, 1,1-diethylpropyloxycarbonyl, 1,1-diethylpropyloxycarbonylmethyl, 1-ethylcyclopentyloxycarbonyl, 1-ethylcyclopentyloxycarbonylmethyl, 1-ethyl-2-cyclopentenyloxycarbonyl, 1-ethyl-2-cyclopentenyloxycarbonylmethyl, 1-ethoxyethoxycarbonylmethyl, 2-tetrahydropyranyloxycarbonylmethyl, and 2-tetrahydrofuranyloxycarbonylmethyl groups.

Examples of the acid labile groups of formula (L3) include 1-methylcyclopentyl, 1-ethylcyclopentyl, 1-n-propylcyclopentyl, 1-isopropylcyclopentyl, 1-n-butylcyclopentyl, 1-sec-butylcyclopentyl, 1-cyclohexylcyclopentyl, 1-(4-methoxy-n-butyl)cyclopentyl, 1-methylcyclohexyl, 1-ethylcyclohexyl, 3-methyl-1-cyclopenten-3-yl, 3-ethyl-1-cyclopenten-3-yl, 3-methyl-1-cyclohexen-3-yl, and 3-ethyl-1-cyclohexen-3-yl groups.

Of the acid labile groups having formula (L4), groups having the following formulas (L4-1) to (L4-4) are preferred.

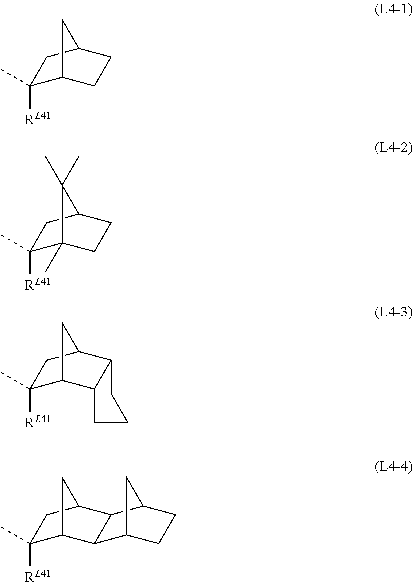

In formulas (L4-1) to (L4-4), the broken line denotes a bonding site and direction. R^{L41} is each independently a monovalent hydrocarbon group, typically a straight, branched or cyclic C₁-C₁₀ alkyl group, such as methyl, ethyl, propyl, isopropyl, n-butyl, sec-butyl, tert-butyl, tert-amyl, n-pentyl, n-hexyl, cyclopentyl and cyclohexyl.

For formulas (L4-1) to (L4-4), there can exist enantiomers and diastereomers. Each of formulae (L4-1) to (L4-4) collectively represents all such stereoisomers. Such stereoisomers may be used alone or in admixture.

For example, the general formula (L4-3) represents one or a mixture of two selected from groups having the following general formulas (L4-3-1) and (L4-3-2).

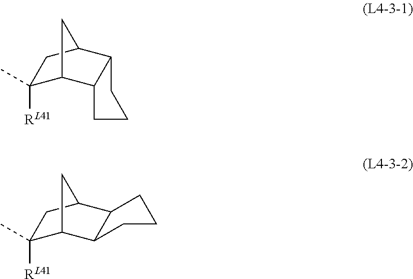

Similarly, the general formula (L4-4) represents one or a mixture of two or more selected from groups having the following general formulas (L4-4-1) to (L4-4-4).

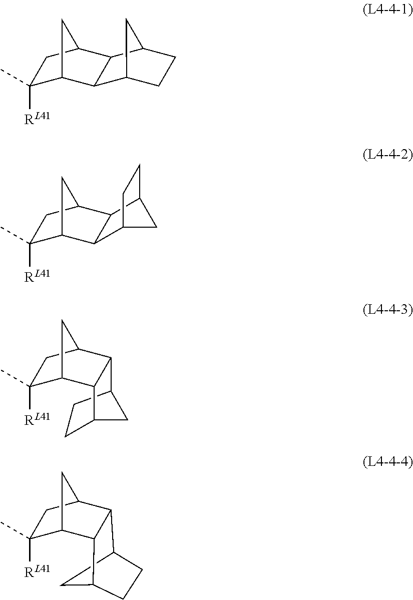

Each of formulas (L4-1) to (L4-4), (L4-3-1) and (L4-3-2), and (L4-4-1) to (L4-4-4) collectively represents an enantiomer thereof and a mixture of enantiomers.

It is noted that in the above formulas (L4-1) to (L4-4), (L4-3-1) and (L4-3-2), and (L4-4-1) to (L4-4-4), the bond direction is on the exo side relative to the bicyclo[2.2.1]heptane ring, which ensures high reactivity for acid catalyzed elimination reaction (see JP-A 2000-336121). In preparing these monomers having a tertiary exo-alkyl group of bicyclo[2.2.1]heptane skeleton as a substituent group, there may be contained monomers substituted with an endo-alkyl group as represented by the following formulas (L4-1-endo) to (L4-4-endo). For good reactivity, an exo proportion of at least 50 mol % is preferred, with an exo proportion of at least 80 mol % being more preferred.

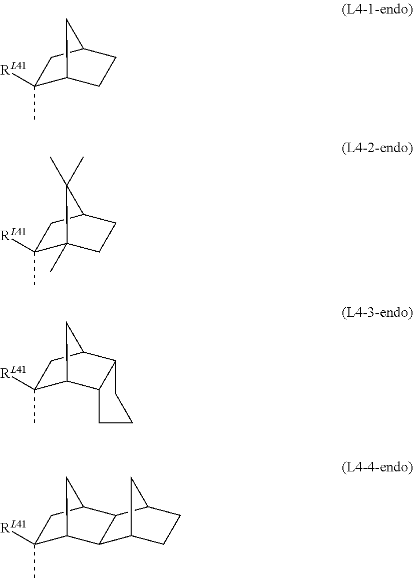

Illustrative examples of the acid labile group of formula (L4) are given below.

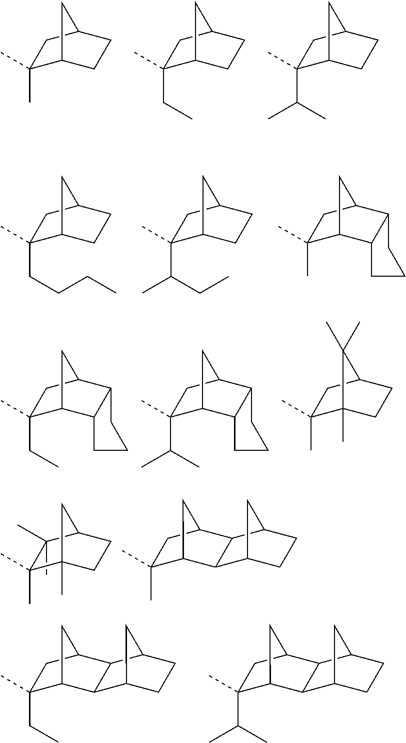

Examples of the tertiary C₄-C₂₀ alkyl groups, trialkylsilyl groups in which each alkyl moiety has 1 to 6 carbon atoms, and C₄-C₂₀ oxoalkyl groups are as exemplified for R^{L04}.

Illustrative examples of the recurring units of formula (3) are given below, but not limited thereto.

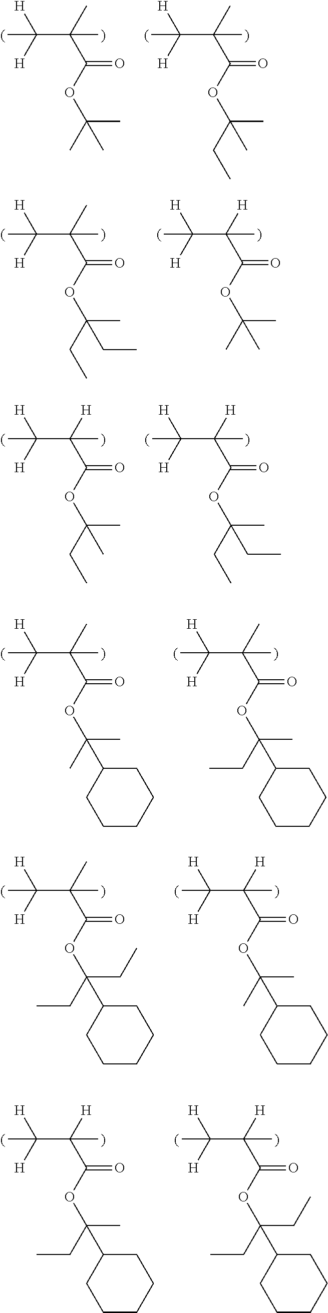

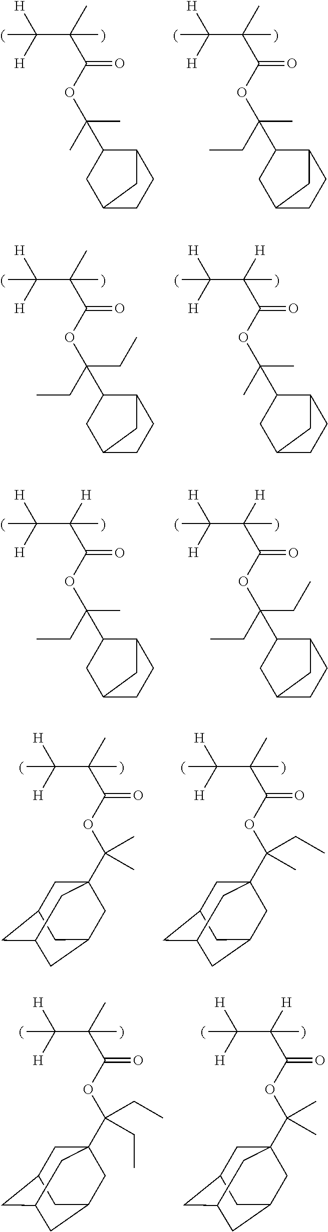

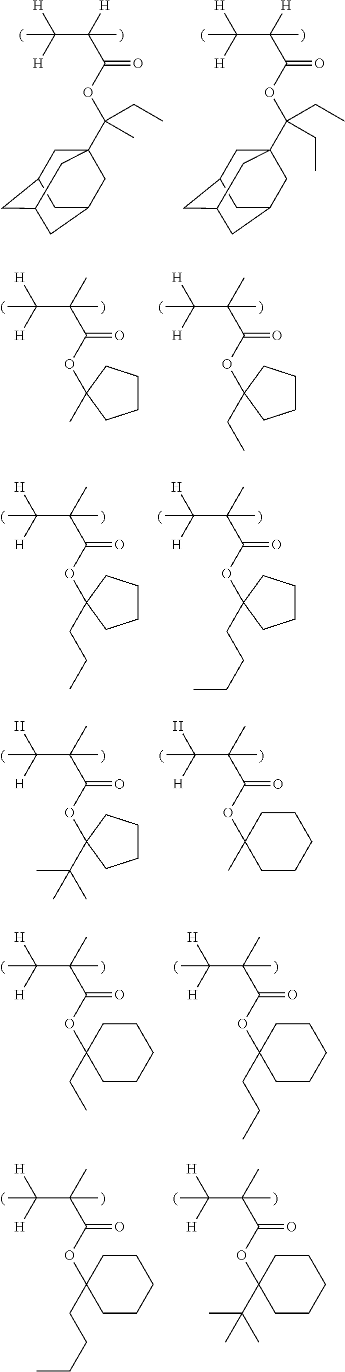

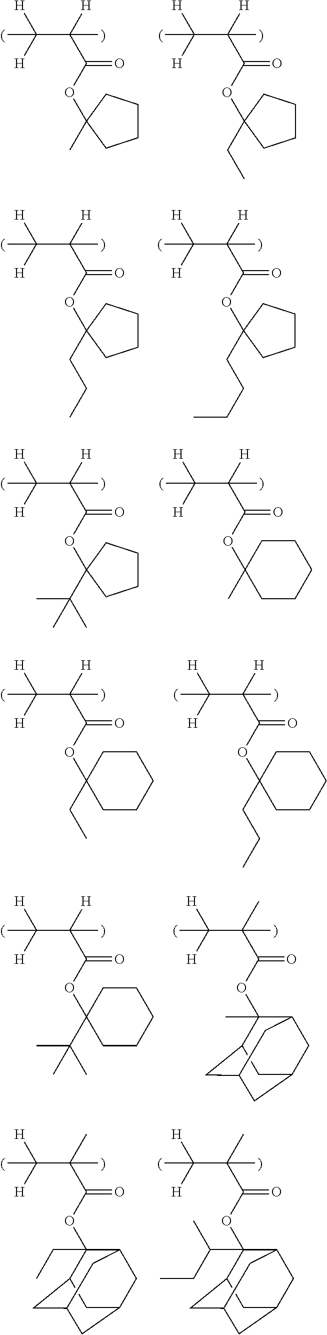

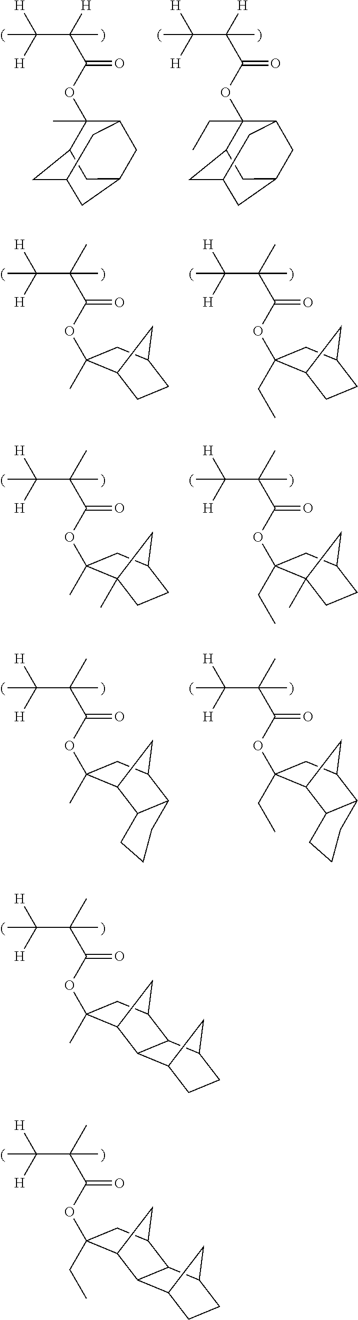

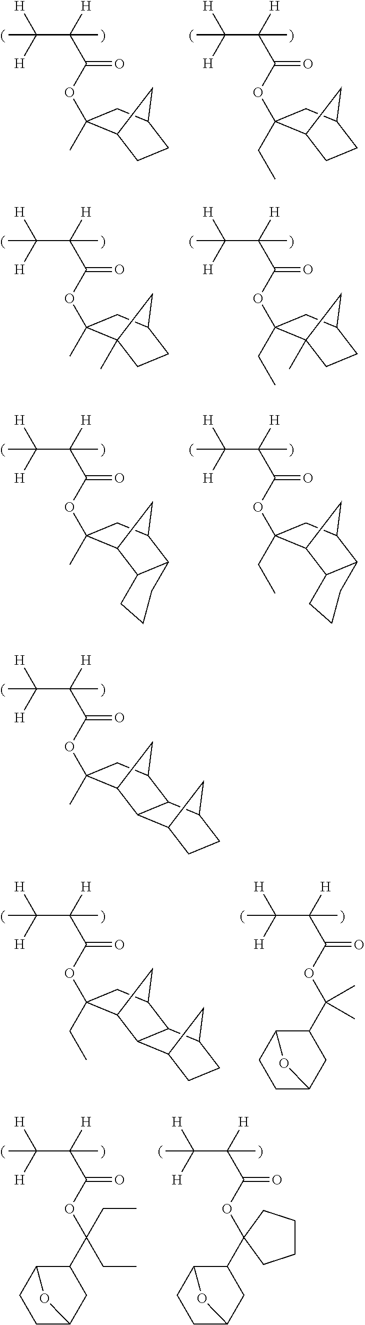

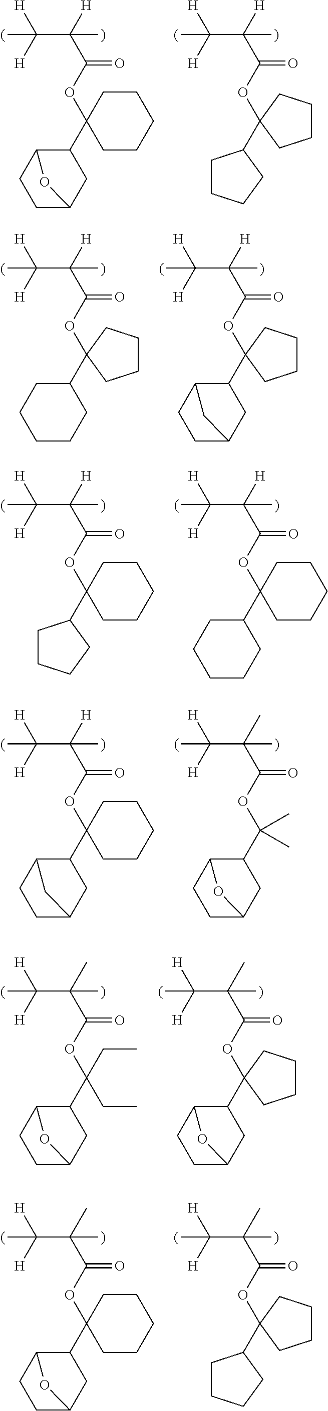

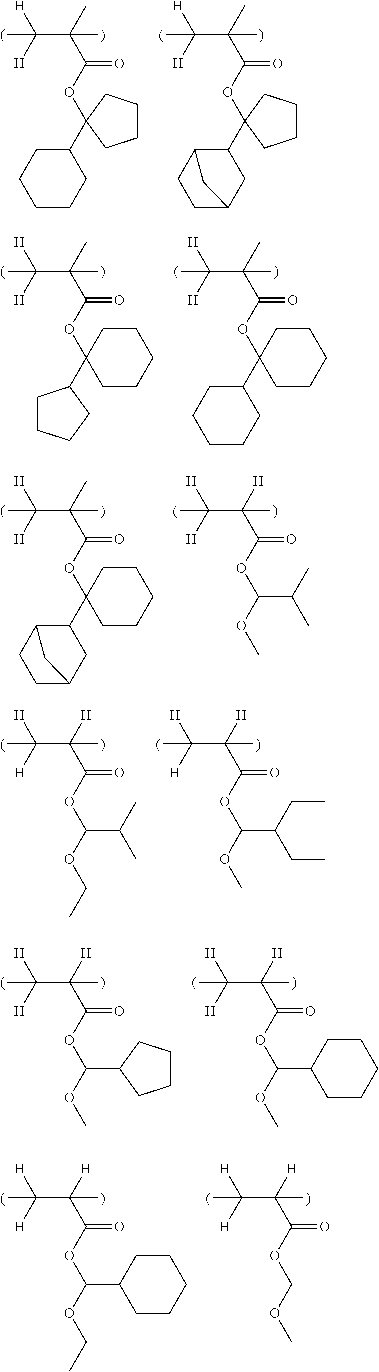

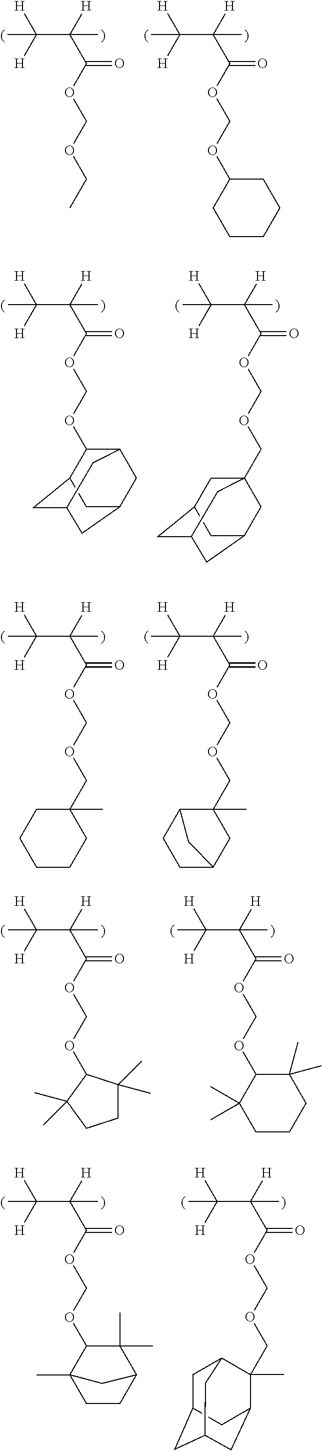

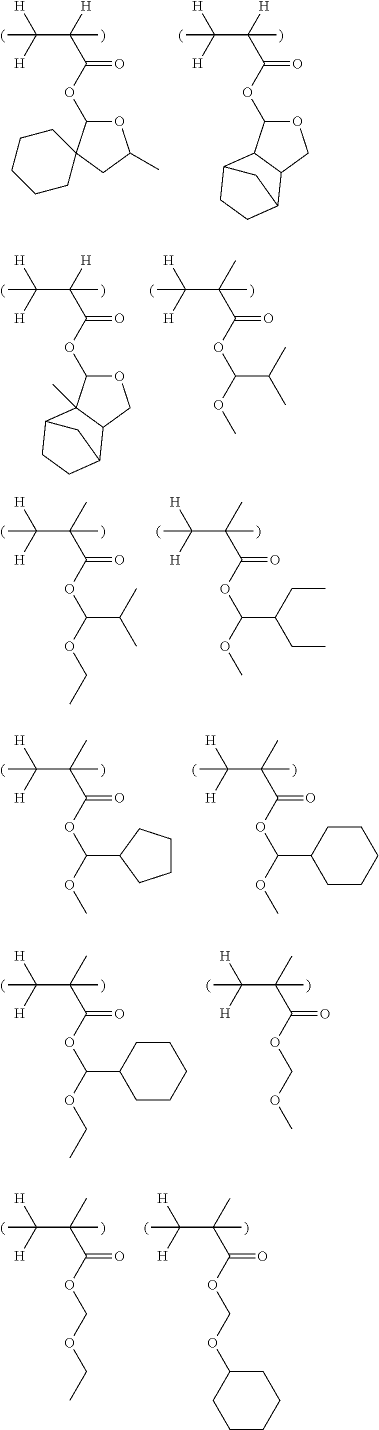

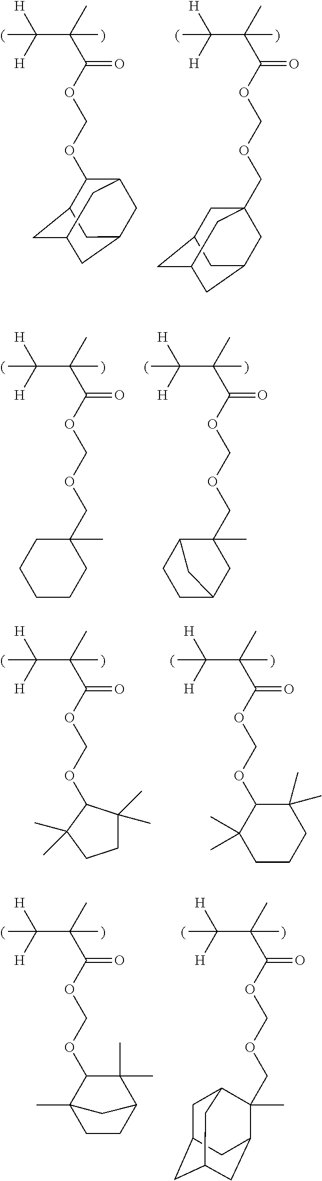

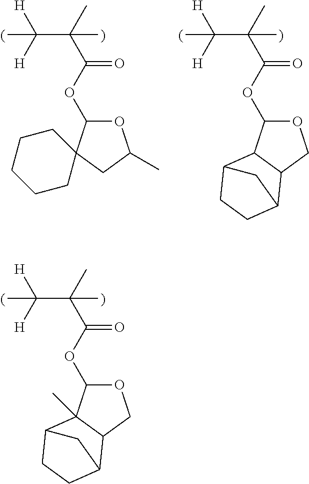

Illustrative examples of the recurring units of formula (4) are given below, but not limited thereto.

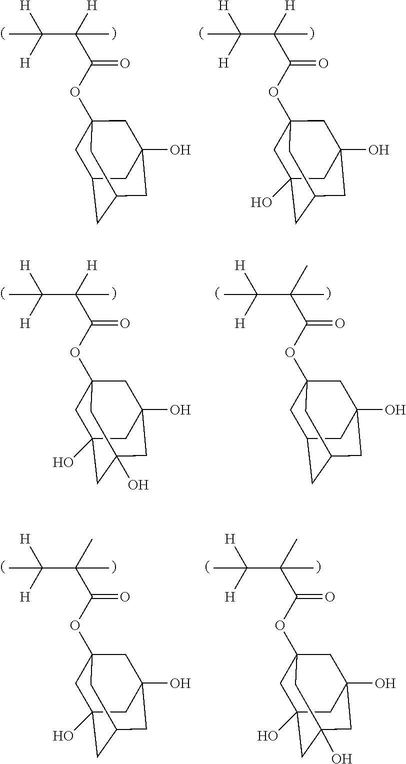

Illustrative examples of the recurring units of formula (5) are given below, but not limited thereto.

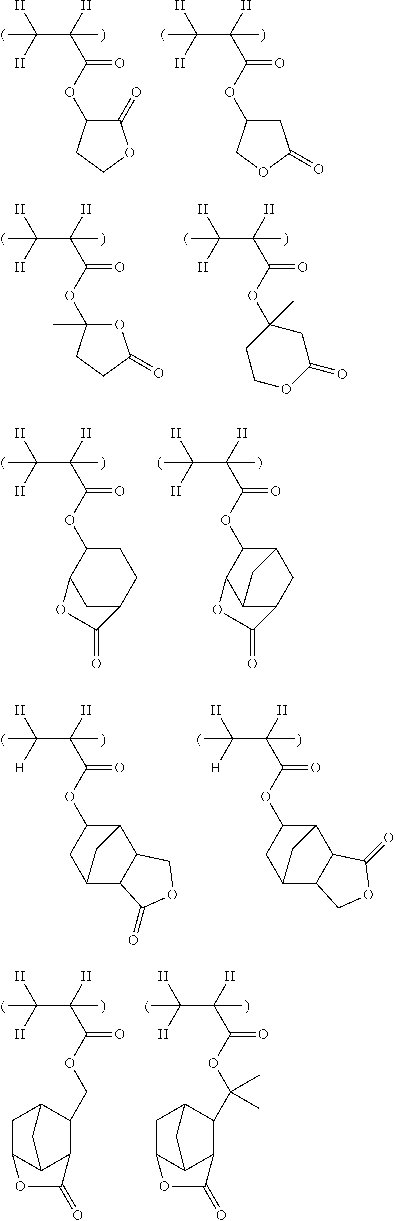

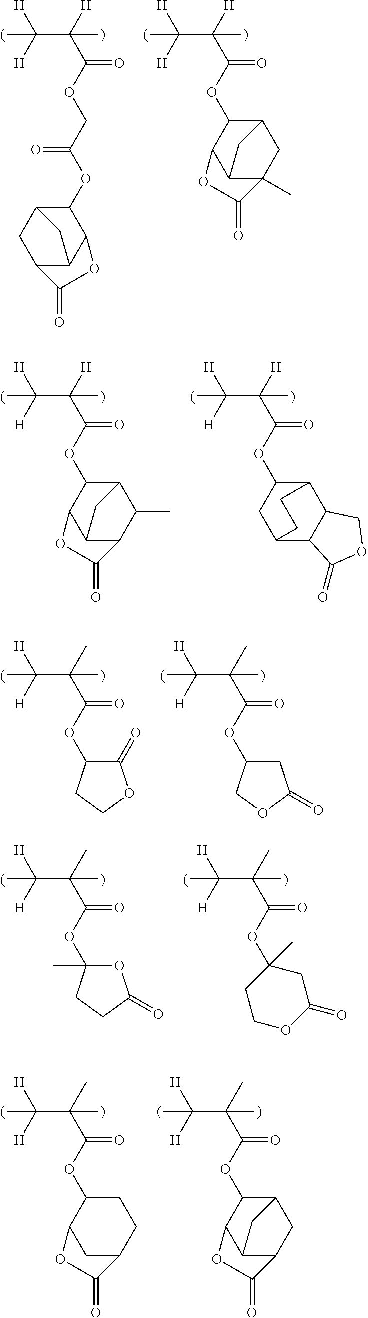

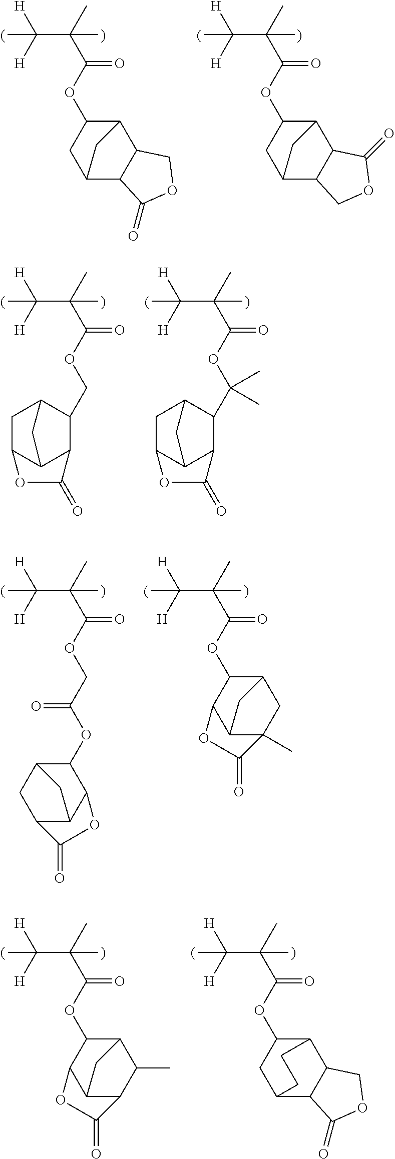

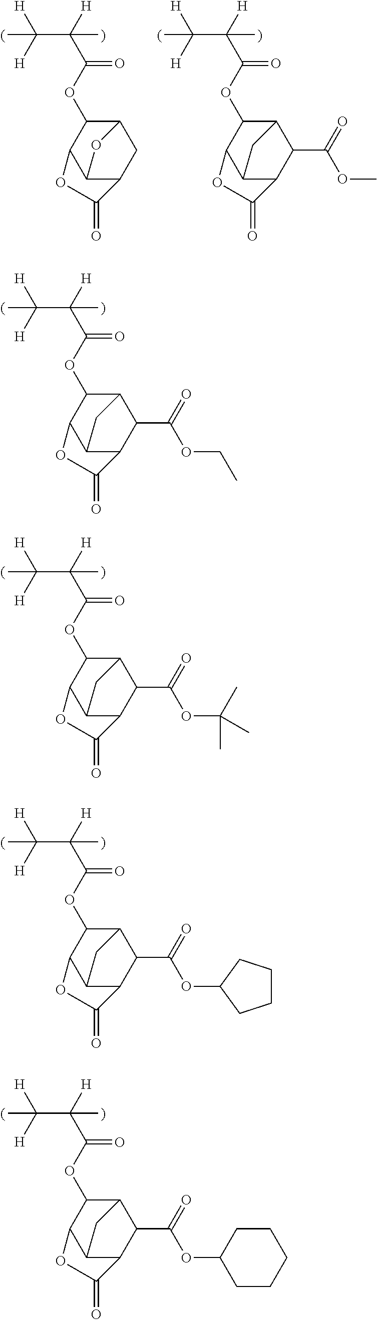

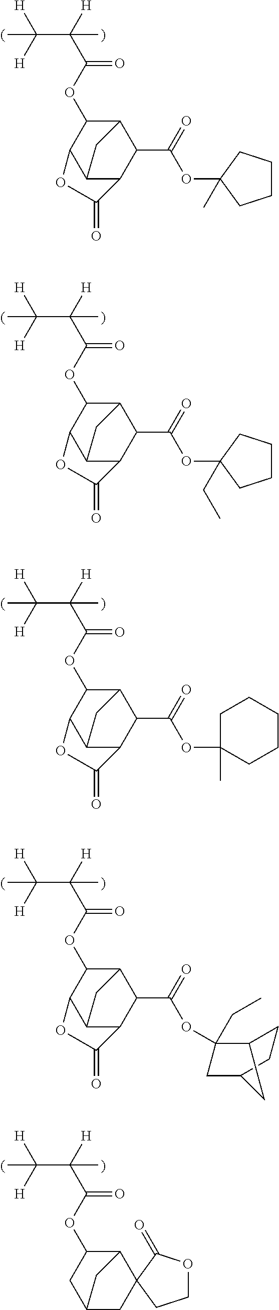

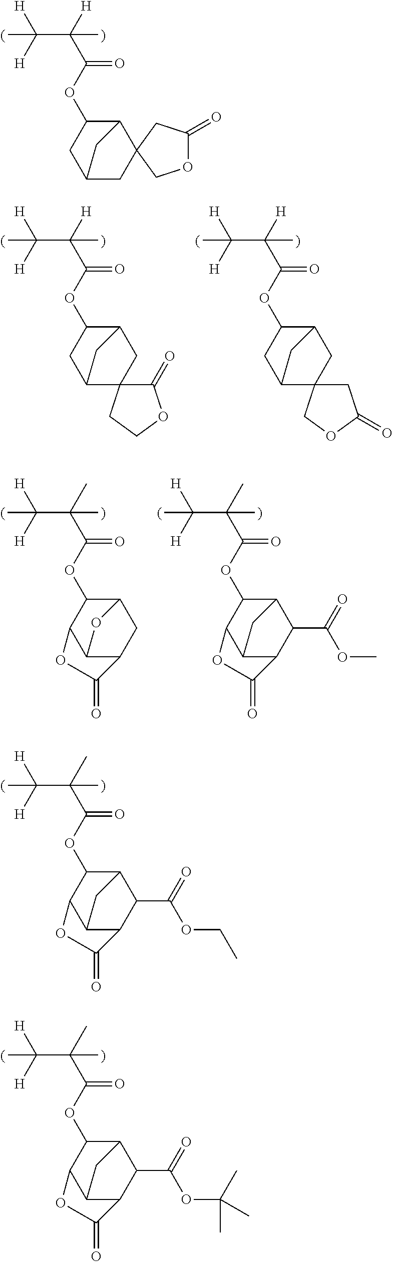

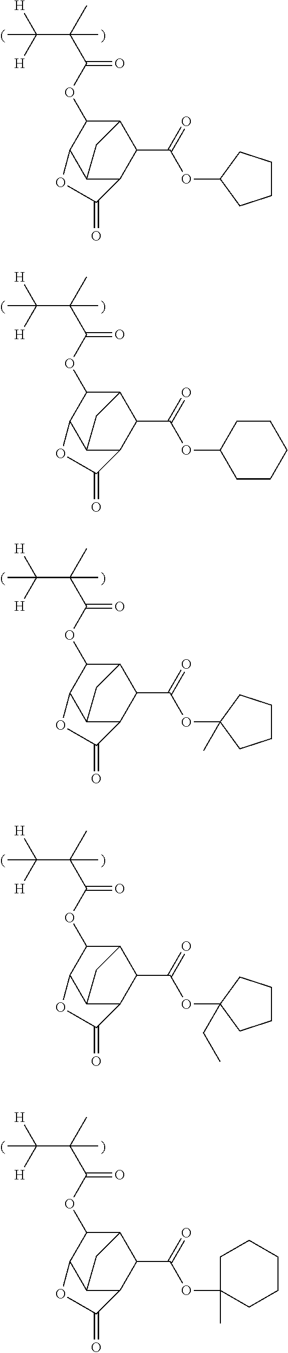

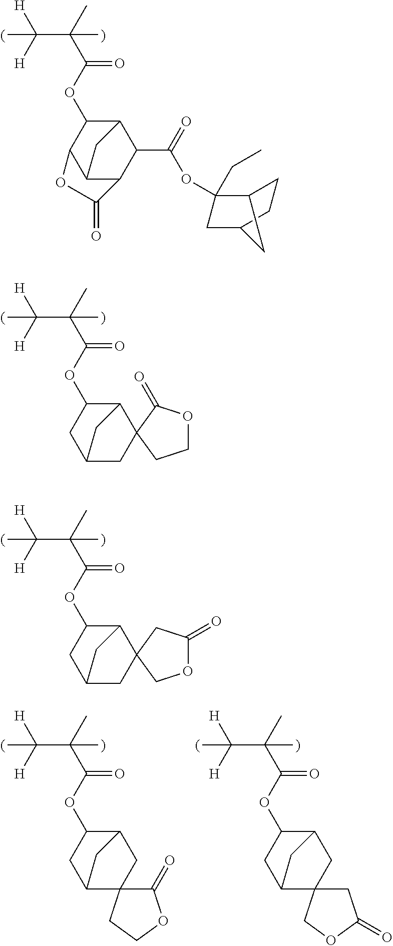

Illustrative examples of the recurring units of formula (6) are given below, but not limited thereto.

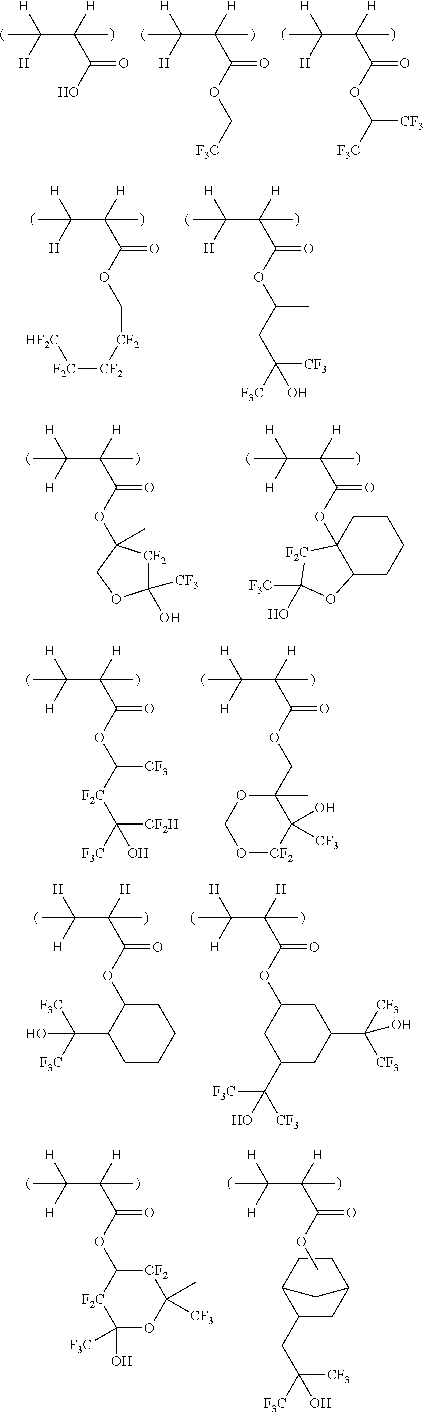

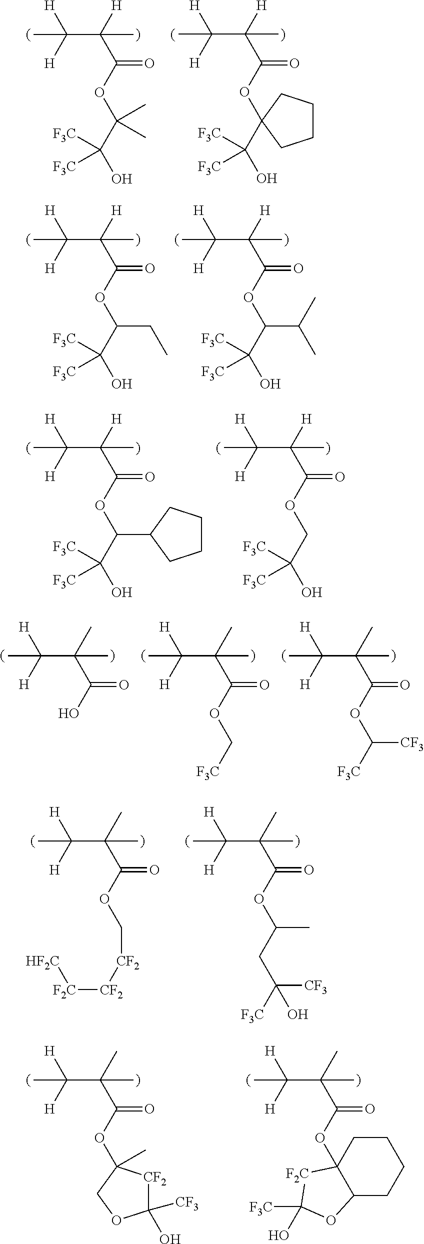

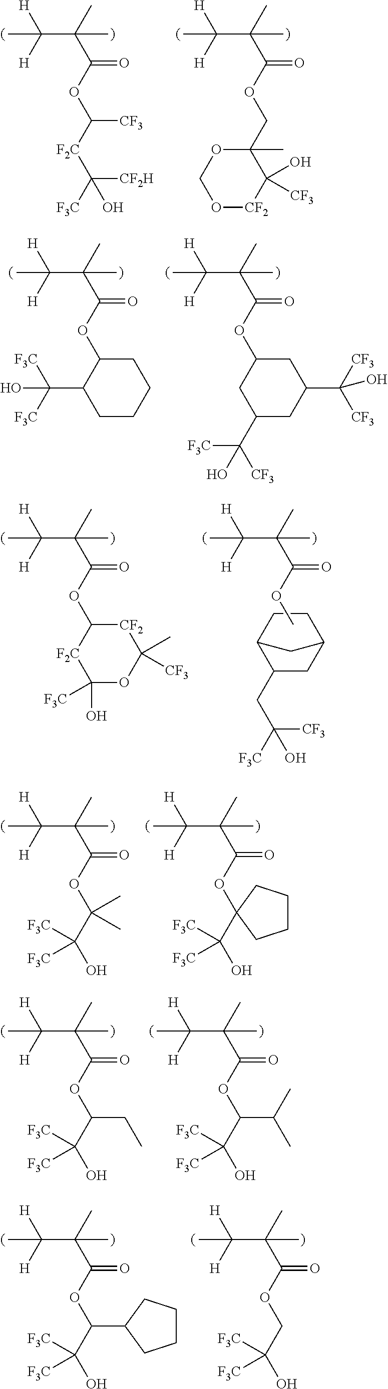

In a preferred embodiment, the polymer used as the base resin in the resist composition has further copolymerized therein units selected from sulfonium salts (d1) to (d3) represented by the general formulae below.

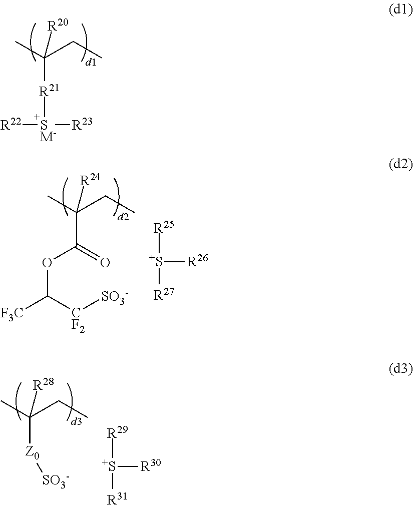

Herein R²⁰, R²⁴, and R²⁸ each are hydrogen or methyl. R²¹ is a single bond, phenylene, —O—R³³—, or —C(═O)—Y—R³³— wherein Y is oxygen or NH, and R³³ is a straight, branched or cyclic C₁-C₆ alkylene group, alkenylene or phenylene group, which may contain a carbonyl (—CO—), ester (—COO—), ether (—O—) or hydroxyl radical. R²², R²³, R²⁵, R²⁶R²⁷, R²⁹, R³⁰, and R³¹ are each independently a straight, branched or cyclic C₁-C₁₂ alkyl group which may contain a carbonyl, ester or ether radical, or a C₆-C₁₂ aryl, C₇-C₂₀ aralkyl, or thiophenyl group. Z₀ is a single bond, methylene, ethylene, phenylene, fluorophenylene, —O—R³²—, or —C(═O)—Z₁—R³²— wherein Z₁ is oxygen or NH, and R³² is a straight, branched or cyclic C₁-C₆ alkylene group, alkenylene or phenylene group, which may contain a carbonyl, ester, ether or hydroxyl radical. M⁻ is a non-nucleophilic counter ion.

The polymer used herein may further comprise recurring units derived from monomers having a carbon-carbon double bond other than the above-described ones, for example, substituted acrylic acid esters such as methyl methacrylate, methyl crotonate, dimethyl maleate and dimethyl itaconate, unsaturated carboxylic acids such as maleic acid, fumaric acid, and itaconic acid, cyclic olefins such as norbornene, norbornene derivatives, and tetracyclo[6.2.1.1^{3,6}.0^{2,7}]dodecene derivatives, unsaturated acid anhydrides such as itaconic anhydride, and other monomers. As the hydrogenated ROMP polymer, those described in JP-A 2003-66612 may be used.

The polymer used herein generally has a weight average molecular weight (Mw) of 1,000 to 500,000, and preferably 3,000 to 100,000, as measured by GPC using polystyrene standards. Outside the range, there may result an extreme drop of etch resistance, and a drop of resolution due to difficulty to gain a dissolution rate difference before and after exposure.

In the polymer (C), appropriate proportions (mol %) of the respective recurring units derived from the monomers are given below although the invention is not limited thereto.

The polymer may contain:

(I) constituent units of one or more types having formula (3) in a proportion of more than 1 mol % to 50 mol %, preferably 5 to 40 mol %, and more preferably 10 to 30 mol %,

(II) constituent units of one or more types having formulas (4) to (6) in a proportion of 50 to 99 mol %, preferably 60 to 95 mol %, and more preferably 70 to 90 mol %,

(III) constituent units of one or more types having formulas (d1) to (d3) in a proportion of 0 to 30 mol %, preferably 0 to 20 mol %, and more preferably 0 to 10 mol %, and

(IV) constituent units of one or more types derived from other monomers in a proportion of 0 to 80 mol %, preferably 0 to 70 mol %, and more preferably 0 to 50 mol %, based on the total moles of constituent units.

The polymers may be used alone or in admixture of two or more. The performance of a resist composition may be adjusted by using two or more polymers.

The organic solvent (D) used herein may be any organic solvent in which the base resin, acid generator, carboxylic acid sulfonium salt, and other components are soluble. Illustrative, non-limiting, examples of the organic solvent include ketones such as cyclohexanone and methyl amyl ketone; alcohols such as 3-methoxybutanol, 3-methyl-3-methoxybutanol, 1-methoxy-2-propanol, and 1-ethoxy-2-propanol; ethers such as propylene glycol monomethyl ether, propylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone, which may be used alone or in combinations of two or more. Of the above organic solvents, it is recommended to use 1-ethoxy-2-propanol, PGMEA, cyclohexanone, γ-butyrolactone, and mixtures thereof because the acid generator is most soluble therein.

An appropriate amount of the organic solvent used is 200 to 5,000 parts, more preferably 400 to 3,000 parts by weight per 100 parts by weight of the base resin.

The resist composition may further comprise one or more of the following components: (E) a quencher, (S) a surfactant which is insoluble or substantially insoluble in water and soluble in alkaline developer, and/or a surfactant which is insoluble or substantially insoluble in water and alkaline developer (hydrophobic resin), and (F) an organic acid derivative and/or fluorinated alcohol. With respect to the quencher (E), the surfactant (S), PAG other than the PAG defined herein, and (F) the organic acid derivative and/or fluorinated alcohol, reference may be made to US 20090274978 (JP-A 2009-269953) and JP-A 2010-215608.

The quencher (E) may be a compound capable of suppressing the rate of diffusion when the acid generated by the PAG diffuses within the resist film. The inclusion of quencher facilitates adjustment of resist sensitivity and holds down the rate of acid diffusion within the resist film, resulting in better resolution. In addition, it suppresses changes in sensitivity following exposure and reduces substrate and environment dependence, as well as improving the exposure latitude and the pattern profile. The inclusion of quencher is also effective for improving adhesion to the substrate.

Examples of suitable quenchers include primary, secondary, and tertiary aliphatic amines, mixed amines, aromatic amines, heterocyclic amines, nitrogen-containing compounds with carboxyl group, nitrogen-containing compounds with sulfonyl group, nitrogen-containing compounds with hydroxyl group, nitrogen-containing compounds with hydroxyphenyl group, alcoholic nitrogen-containing compounds, amide derivatives, imide derivatives, carbamate derivatives, and ammonium salts. Of these, preferred are tertiary amines, amine oxides, benzimidazoles and anilines having a polar functional group such as ether, carbonyl, ester or alcohol.

Preferred tertiary amines include 2-morpholinoethyl esters of straight, branched or cyclic C₂-C₂₀ aliphatic carboxylic acids and trialkylamines having a straight, branched or cyclic C₂-C₁₀ alkyl moiety. Also included are substituted forms of these amines in which some carbon-bonded hydrogen atoms are replaced by hydroxyl groups. These amines may have an ether or ester linkage. Examples include 2-morpholinoethyl 2-methoxyacetate, 2-morpholinoethyl 2-(2-methoxyethoxy)acetate, 2-morpholinoethyl 2-[2-(2-methoxyethoxy)ethoxy]acetate, 2-morpholinoethyl hexanoate, 2-morpholinoethyl octanoate, 2-morpholinoethyl decanoate, 2-morpholinoethyl laurate, 2-morpholinoethyl myristate, 2-morpholinoethyl palmitate, 2-morpholinoethyl stearate, 2-morpholinoethyl cyclohexanecarboxylate, 2-morpholinoethyl adamantanecarboxylate, 4-[2-[(2-methoxyethoxy)methoxy]ethyl]morpholine, 4-[2-[2-(2-methoxyethoxy)ethoxy]ethyl]morpholine, 4-[2-[2-(2-butoxyethoxy)ethoxy]ethyl]morpholine, tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris(2-acetoxyethyl)amine, tris(2-propionyloxyethyl)amine, tris(2-butyryloxyethyl)amine, tris(2-isobutyryloxyethyl)amine, tris(2-valeryloxyethyl)amine, and tris(2-pivaloyloxyethyl)amine.

Preferred examples of the benzimidazoles include benzimidazole, 2-phenylbenzimidazole, 1-(2-acetoxyethoxy)benzimidazole, 1-[2-(methoxymethoxy)ethyl]benzimidazole, 1-[2-(methoxymethoxy)ethyl]-2-phenylbenzimidazole, and 1-(2-(2-(2-methoxyethoxy)ethoxy)ethyl)benzimidazole.

Preferred examples of the anilines include aniline, N-methylaniline, N-ethylaniline, N-propylaniline, N,N-dimethylaniline, N,N-bis(hydroxyethyl)aniline, 2-methylaniline, 3-methylaniline, 4-methylaniline, ethylaniline, propylaniline, dimethylaniline, 2,6-diisopropylaniline, trimethylaniline, 2-nitroaniline, 3-nitroaniline, 4-nitroaniline, 2,4-dinitroaniline, 2,6-dinitroaniline, 3,5-dinitroaniline, and N,N-dimethyltoluidine.

Also included are primary or secondary amines protected with tert-butoxycarbonyl (tBOC). Those compounds described in JP-A 2007-298569 and JP-A 2010-20204 are also useful.

The quenchers may be used alone or in admixture of two or more. The quencher is preferably used in an amount of 0.001 to 8 parts, more preferably 0.01 to 4 parts by weight per 100 parts by weight of the base resin. Less than 0.001 part of the quencher may achieve no addition effect whereas more than 8 parts may lead to too low a sensitivity.

To the resist composition, the surfactant (S) may be added. Reference should be made to those compounds defined as component (S) in JP-A 2010-215608 and JP-A 2011-16746.

While many examples of the surfactant which is insoluble or substantially insoluble in water and alkaline developer are described in these patent documents, preferred examples are FC-4430, Surflon S-381, Surfynol E1004, KH-20 and KH-30, which may be used alone or in admixture. Partially fluorinated oxetane ring-opened polymers having the structural formula (surf-1) are also useful.

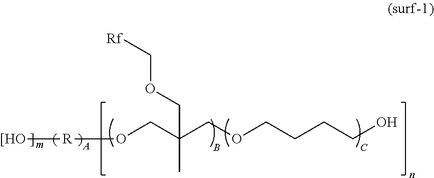

It is provided herein that R, Rf, A, B, C, m, and n are applied to only formula (surf-1), independent of their descriptions other than for the surfactant. R is a di- to tetra-valent C₂-C₅ aliphatic group. Exemplary divalent groups include ethylene, 1,4-butylene, 1,2-propylene, 2,2-dimethyl-1,3-propylene and 1,5-pentylene. Exemplary tri- and tetra-valent groups are shown below.

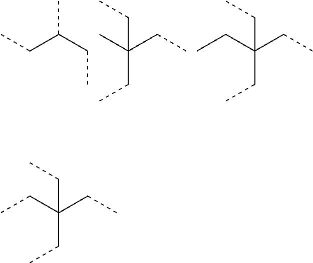

Herein the broken line denotes a valence bond. These formulae are partial structures derived from glycerol, trimethylol ethane, trimethylol propane, and pentaerythritol, respectively. Of these, 1,4-butylene and 2,2-dimethyl-1,3-propylene are preferably used.

Rf is trifluoromethyl or pentafluoroethyl, and preferably trifluoromethyl. The letter m is an integer of 0 to 3, n is an integer of 1 to 4, and the sum of m and n, which represents the valence of R, is an integer of 2 to 4. A is equal to 1, B is an integer of 2 to 25, and C is an integer of 0 to 10. Preferably, B is an integer of 4 to 20, and C is 0 or 1. Note that the above structural formula does not prescribe the arrangement of respective constituent units while they may be arranged either in blocks or randomly. For the preparation of surfactants in the form of partially fluorinated oxetane ring-opened polymers, reference should be made to U.S. Pat. No. 5,650,483, for example.

The surfactant which is insoluble or substantially insoluble in water and soluble in alkaline developer is useful when ArF immersion lithography is applied to the resist composition in the absence of a resist protective film. In this embodiment, the surfactant has a propensity to segregate on the resist surface after spin coating for achieving a function of minimizing water penetration or leaching. The surfactant is also effective for preventing water-soluble components from being leached out of the resist film for minimizing any damage to the exposure tool. The surfactant becomes solubilized during alkaline development following exposure and PEB, and thus forms few or no foreign particles which become defects. The preferred surfactant is a polymeric surfactant which is insoluble or substantially insoluble in water, but soluble in alkaline developer, also referred to as “hydrophobic resin” in this sense, and especially which is water repellent and enhances water slippage. Suitable polymeric surfactants are shown below.

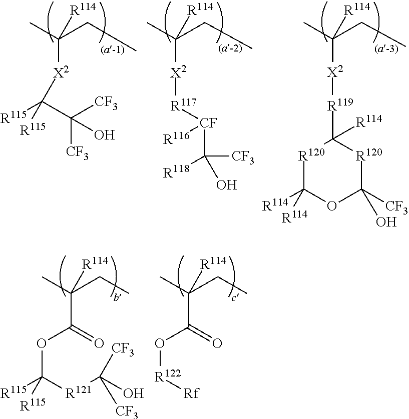

Herein R¹¹⁴ is each independently hydrogen, fluorine, methyl or trifluoromethyl. R¹¹⁵ is each independently hydrogen or a straight, branched or cyclic C₁-C₂₀ alkyl or fluoroalkyl group, or two R¹¹⁵ in a common monomer may bond together to form a ring with the carbon atom to which they are attached, and in this event, they together represent a straight, branched or cyclic C₂-C₂₀ alkylene or fluoroalkylene group. R¹¹⁶ is fluorine or hydrogen, or R¹¹⁶ may bond with R¹¹⁷ to form a non-aromatic ring of 3 to 10 carbon atoms in total with the carbon atom to which they are attached. R¹¹⁷ is a straight, branched or cyclic C₁-C₆ alkylene group in which at least one hydrogen atom may be substituted by a fluorine atom. R¹¹⁸ is a straight or branched C₁-C₁₀ alkyl group in which at least one hydrogen atom is substituted by a fluorine atom. Alternatively, R¹¹⁷ and R¹¹⁸ may bond together to form a non-aromatic ring with the carbon atoms to which they are attached. In this event, R¹¹⁷, R¹¹⁸ and the carbon atoms to which they are attached together represent a trivalent organic group of 2 to 12 carbon atoms in total. R¹¹⁹ is a single bond or a C₁-C₄ alkylene. R¹²⁰ is each independently a single bond, —O—, or —CR¹¹⁴R¹¹⁴—. R¹²¹ is a straight or branched C₁-C₄ alkylene group, or may bond with R¹¹⁵ within a common monomer to form a C₃-C₆ non-aromatic ring with the carbon atom to which they are attached. R¹²² is 1,2-ethylene, 1,3-propylene, or 1,4-butylene. Rf is a linear perfluoroalkyl group of 3 to 6 carbon atoms, typically 3H-perfluoropropyl, 4H-perfluorobutyl, 5H-perfluoropentyl, or 6H-perfluorohexyl. X² is each independently —C(═O)—O—, —O—, or —C(═O)—R¹²³—C(═O)—O—. R¹²³ is a straight, branched or cyclic C₁-C₁₀ alkylene group. The subscripts are in the range: 0≦(a′-1)<1, 0≦(a′-2)<1, 0≦(a′-3)<1, 0<(a′-1)+(a′-2)+(a′-3)<1, 0≦b′<1, 0≦c′<1, and 0<(a′-1)+(a′-2)+(a′-3)+b′+c′≦1.

For the surfactant which is insoluble or substantially insoluble in water and soluble in alkaline developer, reference may be made to JP-A 2008-122932, 2010-134012, 2010-107695, 2009-276363, 2009-192784, 2009-191151, 2009-98638, 2011-250105, and 2011-42789.

The polymeric surfactant preferably has a Mw of 1,000 to 50,000, more preferably 2,000 to 20,000 as measured by GPC versus polystyrene standards. A surfactant with a Mw outside the range may be less effective for surface modification and cause development defects. The polymeric surfactant is preferably formulated in an amount of 0.001 to 20 parts, and more preferably 0.01 to 10 parts by weight per 100 parts by weight of the base resin. Reference should also be made to JP-A 2010-215608.

To the resist composition, a compound which is decomposed with an acid to generate another acid, that is, acid amplifier compound may be added. For these compounds, reference should be made to JP-A 2009-269953 and 2010-215608. In the resist composition, an appropriate amount of the acid amplifier compound is up to 2 parts, and especially up to 1 part by weight per 100 parts by weight of the base resin. Excessive amounts of the acid amplifier compound make diffusion control difficult, leading to degradation of resolution and pattern profile.

Optionally, an organic acid derivative or a compound having a Mw of up to 3,000 which changes its solubility in alkaline developer under the action of an acid, also referred to as dissolution inhibitor, may be added. Reference may be made to JP-A 2009-269953 and 2010-215608.

In a further aspect, the invention provides a chemically amplified positive resist composition for ArF immersion lithography comprising the foregoing components and a surfactant having the general formula (1a).

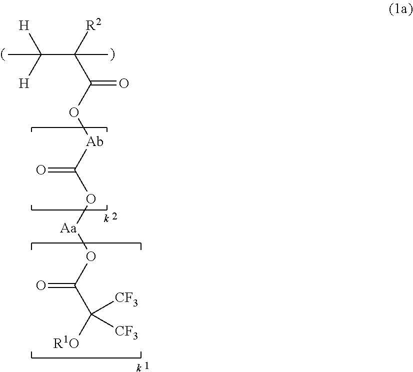

Herein R¹ is hydrogen or a straight, branched or cyclic C₁-C₂₀ monovalent hydrocarbon group in which a constituent moiety —CH₂— may be replaced by —O— or —C(═O)—, R² is hydrogen, fluorine, methyl or trifluoromethyl, Aa is a straight, branched or cyclic C₁-C₂₀ hydrocarbon or fluorinated hydrocarbon group having a valence of k¹+1, Ab is a straight, branched or cyclic C₁-C₆ divalent hydrocarbon group, k¹ is an integer of 1 to 3, and k² is 0 or 1.

The monovalent hydrocarbon group of R¹ may be selected from protective groups for alcoholic hydroxyl group, for example, groups of formulae (L1) and (L2), tertiary alkyl groups of 4 to 20 carbon atoms, preferably 4 to 15 carbon atoms, trialkylsilyl groups wherein each alkyl moiety has 1 to 6 carbon atoms, and oxoalkyl groups of 4 to 20 carbon atoms, as described in conjunction with the acid labile group in component (C). Notably, the groups of formula (L2) wherein y=0 are excluded herein.

Examples of the (k¹+1)-valent, straight, branched or cyclic C₁-C₂₀ hydrocarbon group of Aa are given below.

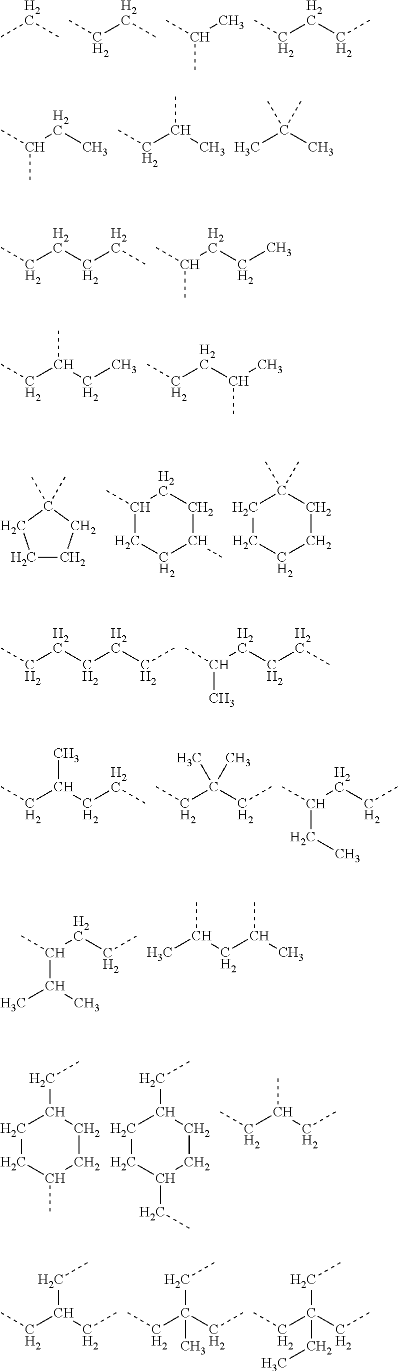

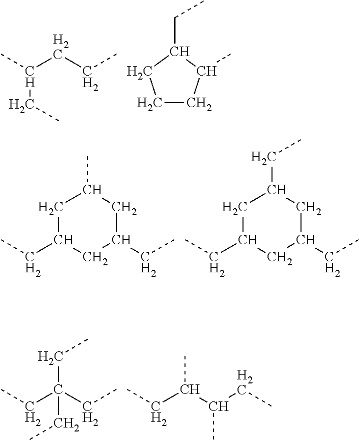

Examples of the (k¹+1)-valent, straight, branched or cyclic C₁-C₂₀ fluorinated hydrocarbon group of Aa include the foregoing hydrocarbon groups in which some or all hydrogen atoms are substituted by fluorine atoms.

Examples of the divalent, straight, branched or cyclic C₁-C₆ hydrocarbon group of Ab are given below.

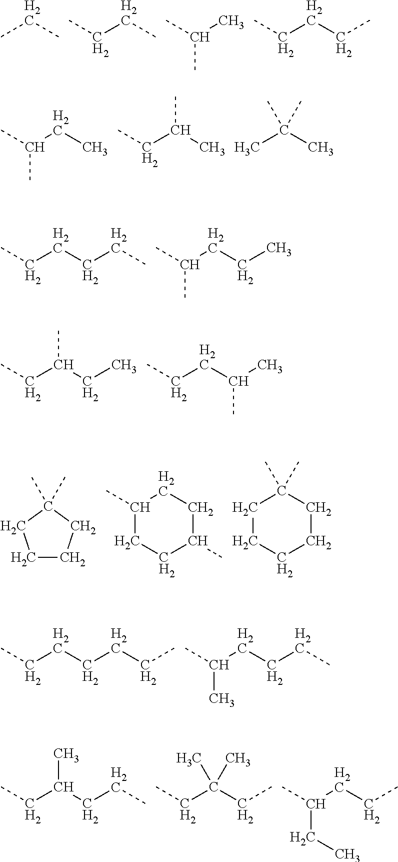

Examples of suitable monomers from which recurring units of formula (1a) are derived are given below.

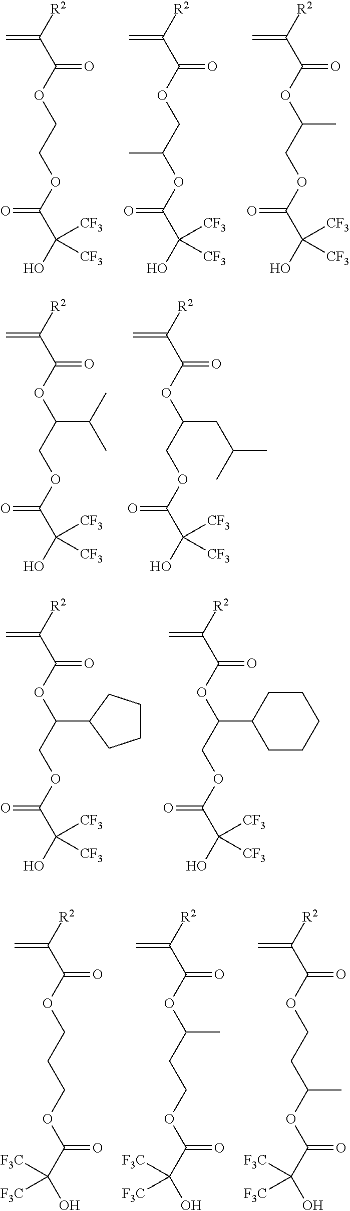

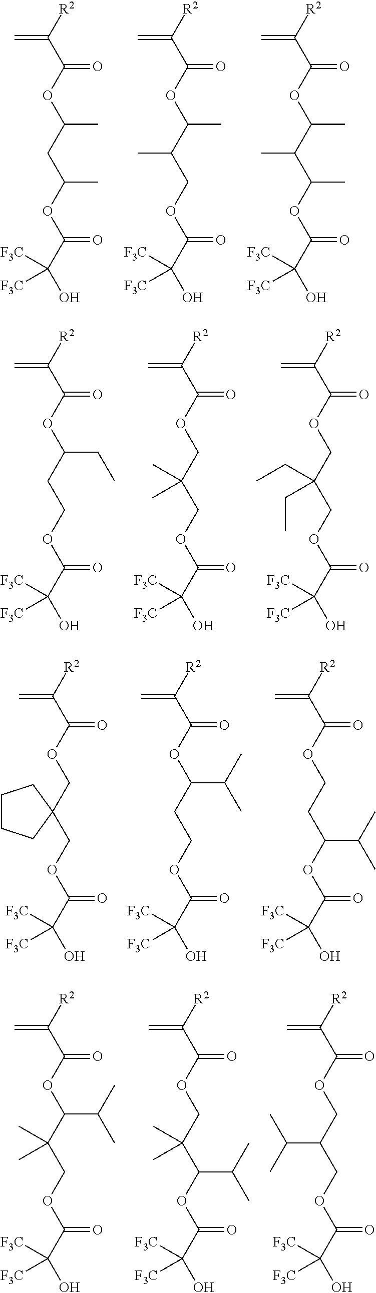

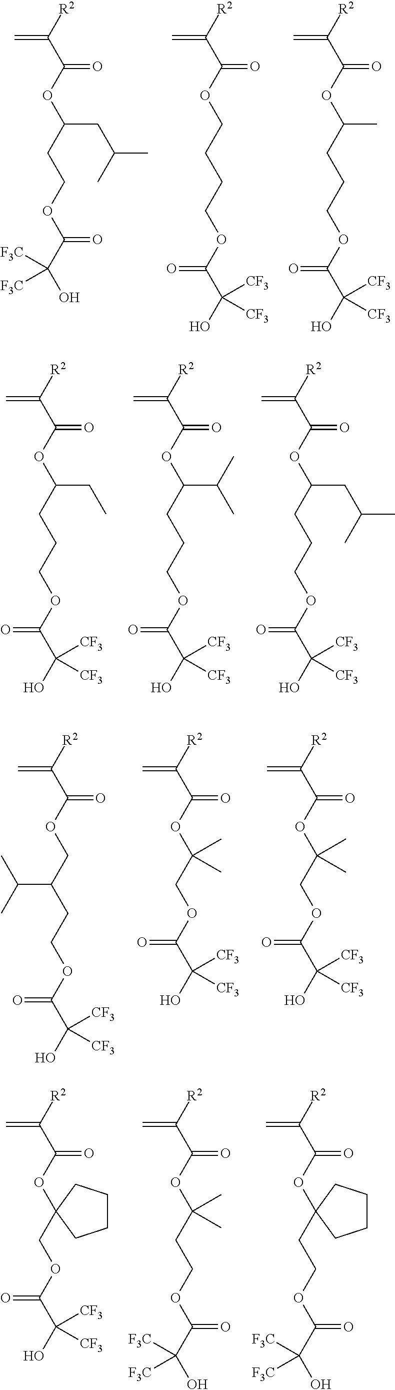

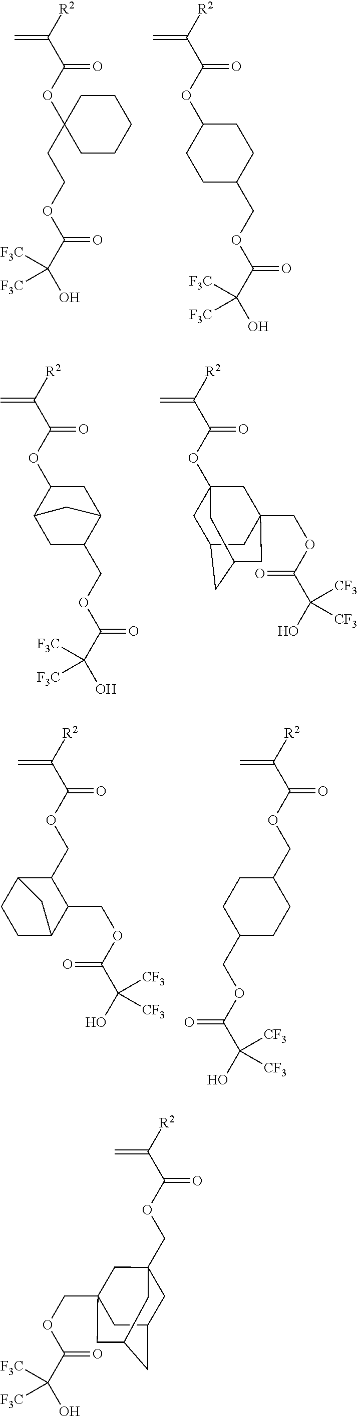

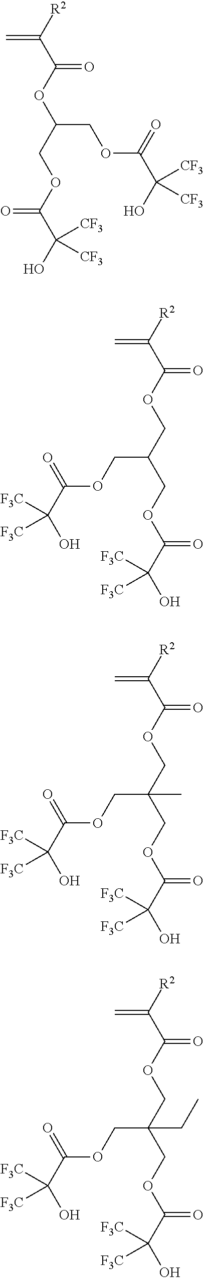

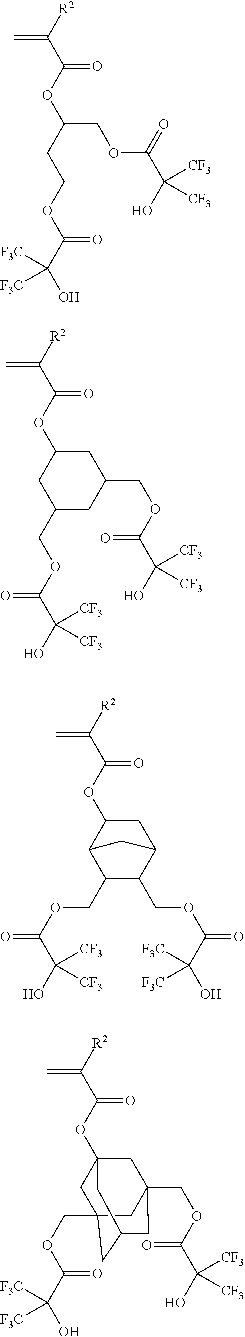

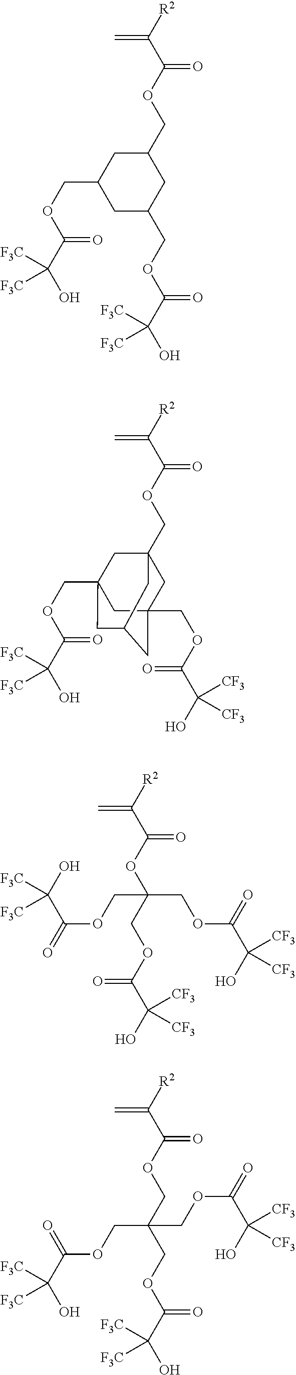

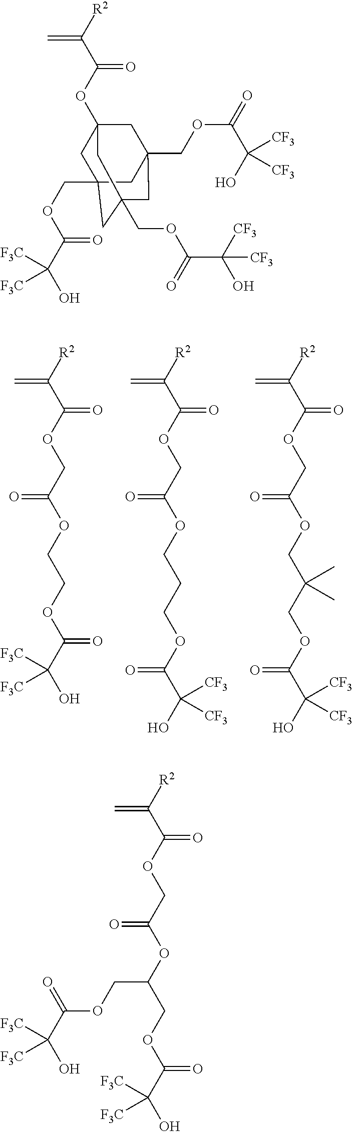

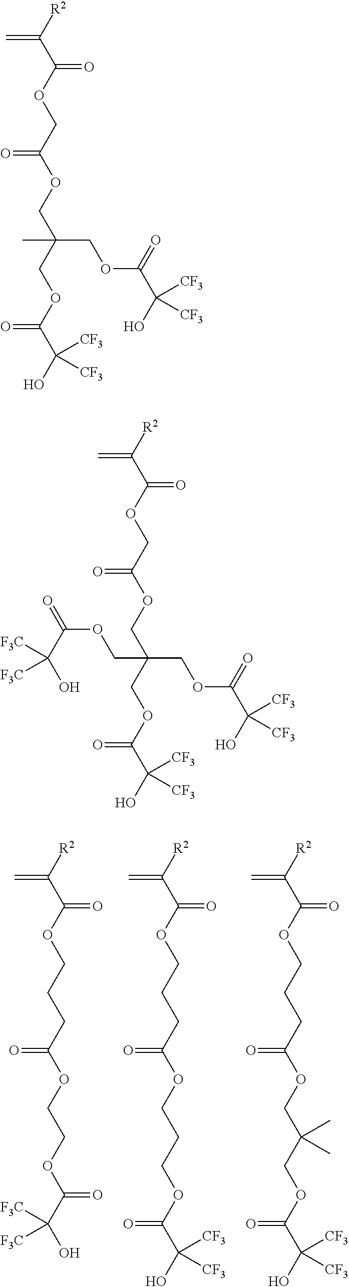

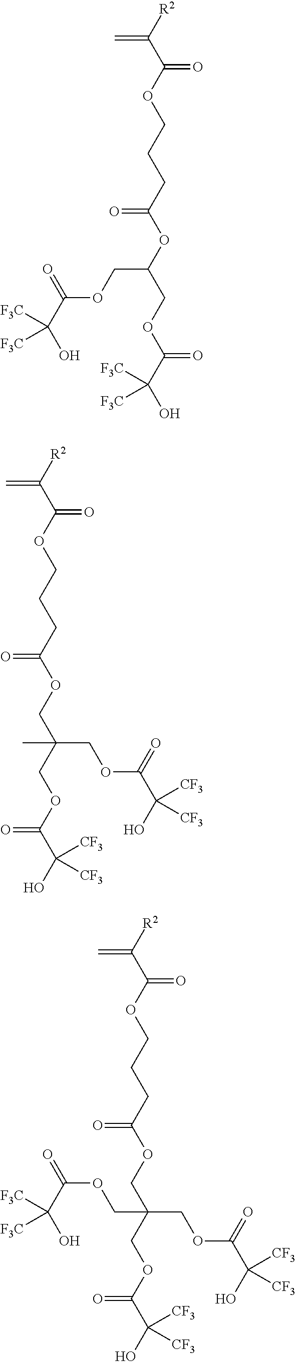

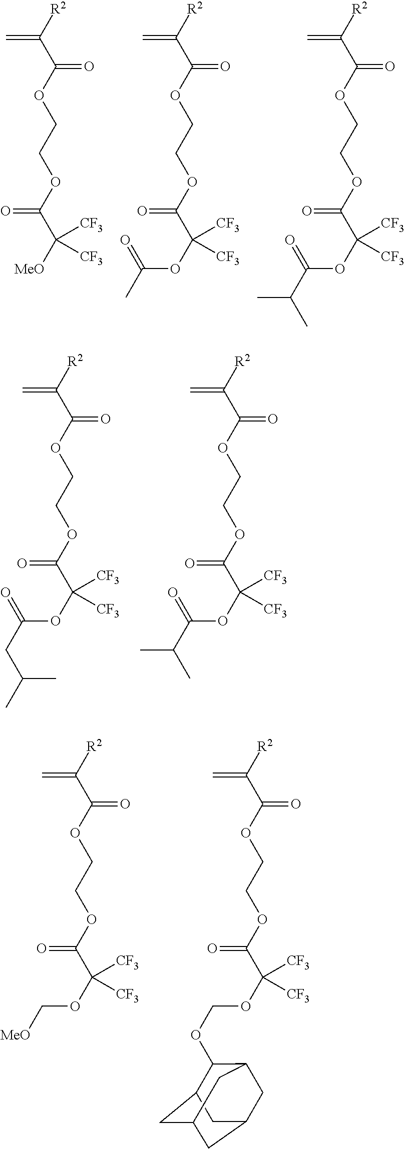

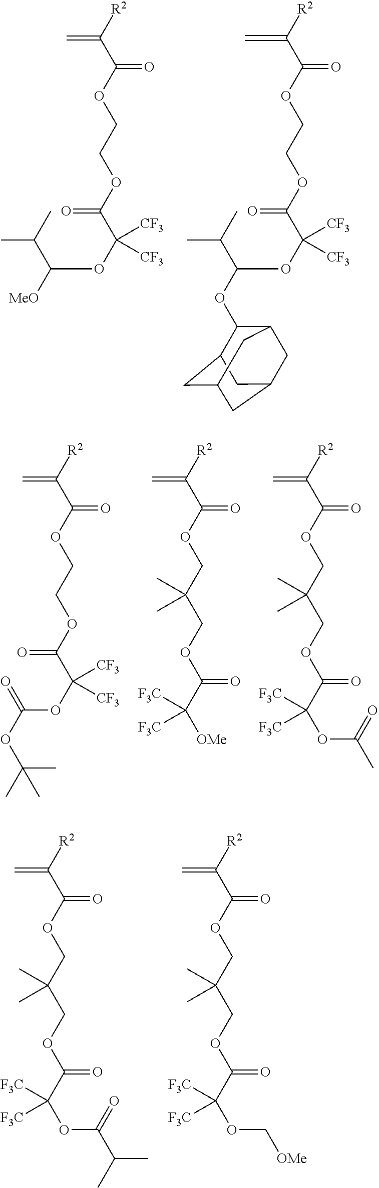

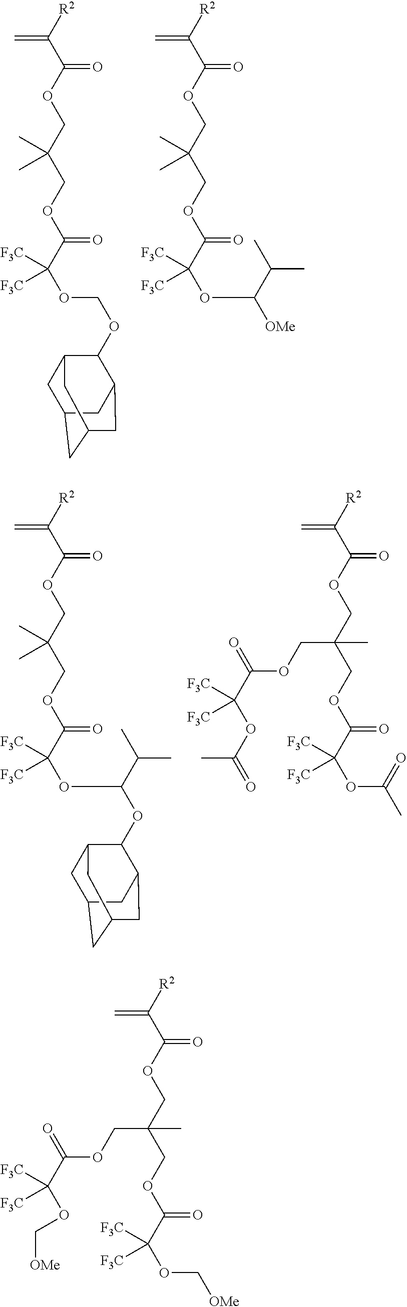

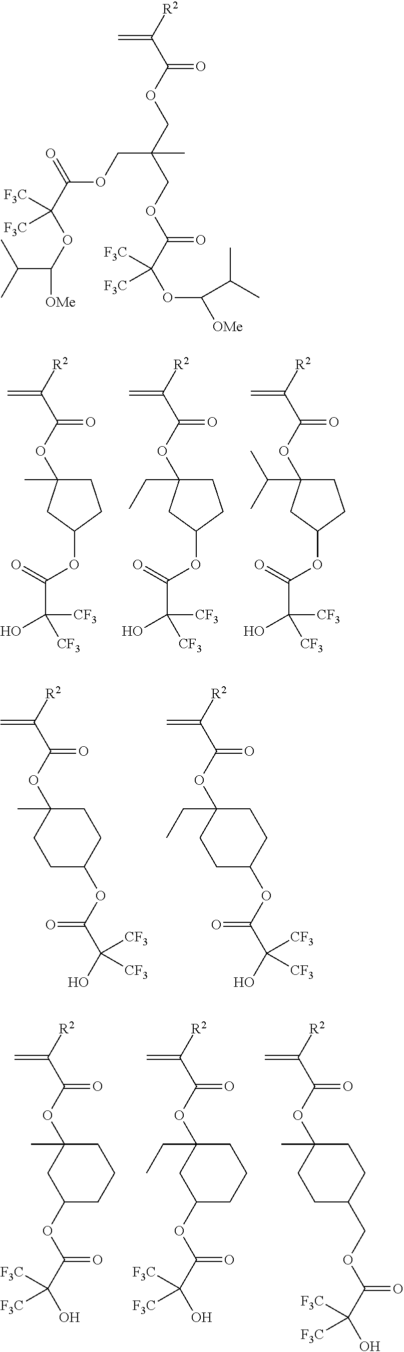

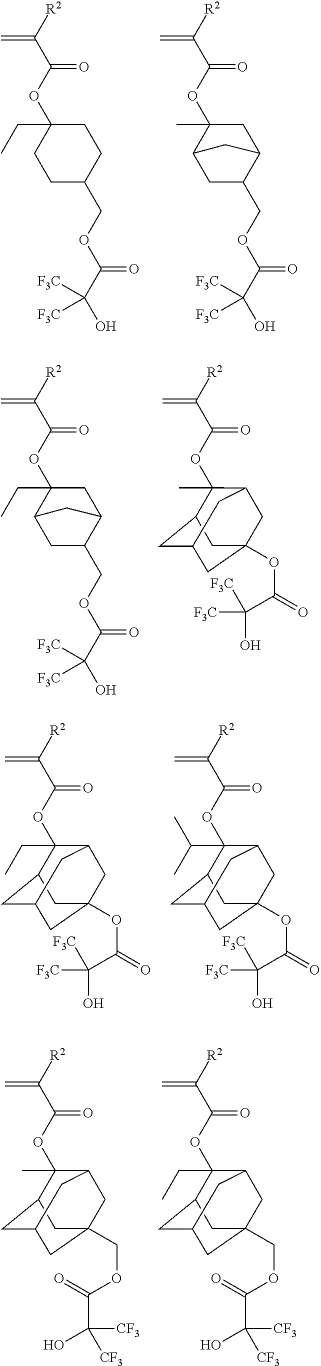

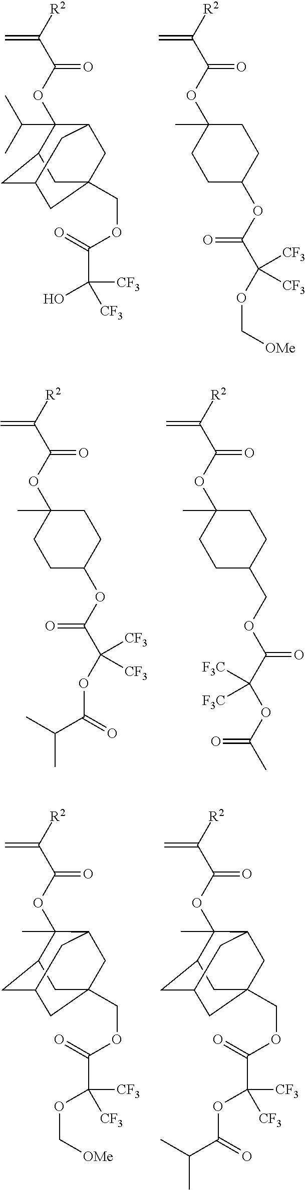

Herein R² is as defined above, and Me stands for methyl.

These compounds are monomers of recurring units having formula (1a). In practice, a monomer polymerizes by itself or with another monomer at its polymerizable moiety (acryloyl or methacryloyl) to form a homopolymer or copolymer.

Also useful is a polymer comprising recurring units of one or more type selected from the general formulae (2a) to (2j) in addition to the recurring units having formula (1a).

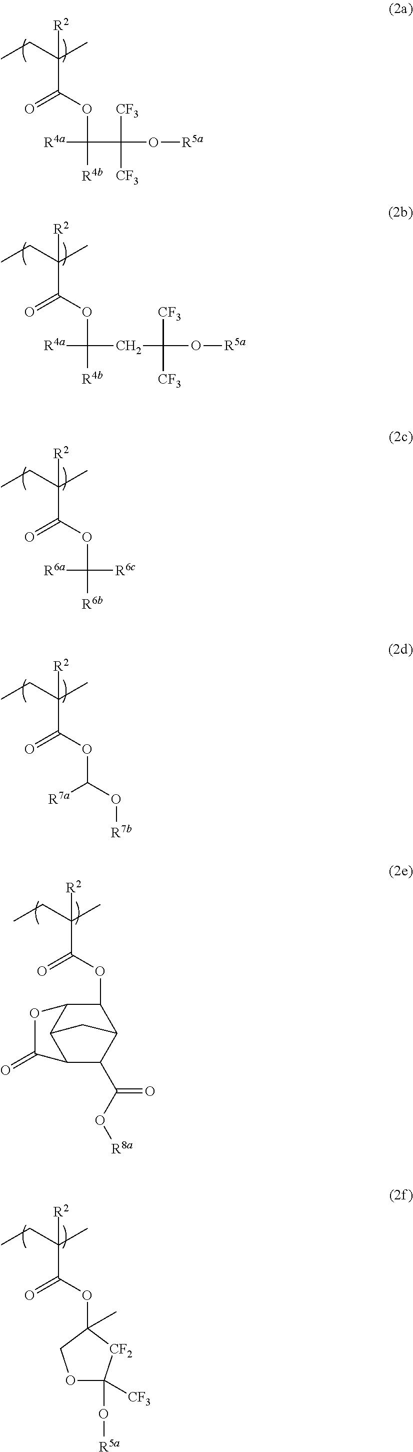

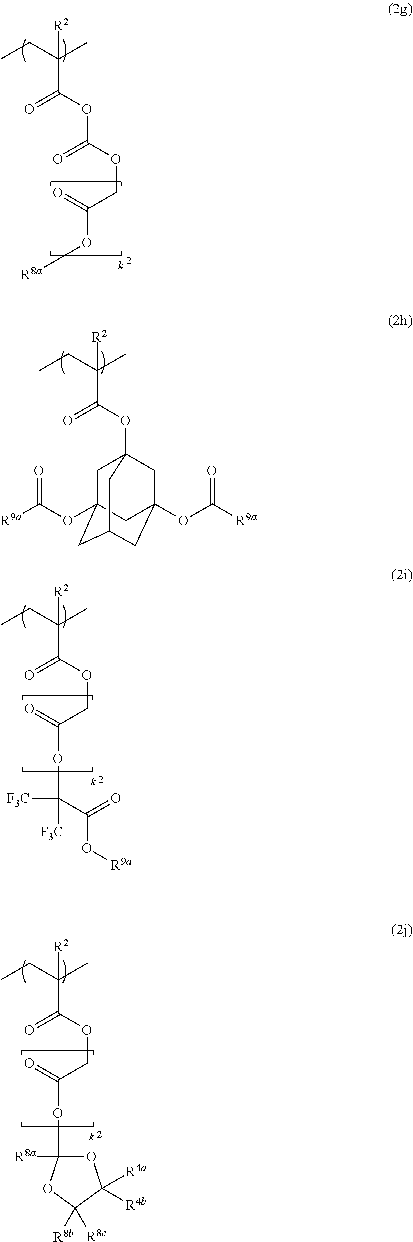

Herein R² is as defined above. R^{4a} and R^{4b} are each independently hydrogen or a straight, branched or cyclic C₁-C₁₅ monovalent hydrocarbon group, typically alkyl, or R^{4a} and R^{4b} may bond together to form a non-aromatic ring of 3 to 8 carbon atoms with the carbon atom to which they are attached. R^{5a} is hydrogen, a straight, branched or cyclic C₁-C₁₅ monovalent hydrocarbon or fluorinated hydrocarbon group, typically alkyl or fluoroalkyl, or an acid labile group, in the case of hydrocarbon group, a constituent moiety —CH₂— may be replaced by —O— or —C(═O)—. R^{6a}, R^{6b} and R^{6c} are each independently hydrogen, or a straight, branched or cyclic C₁-C₁₅ monovalent hydrocarbon group, typically alkyl, or R^{6a} and R^{6b}, R^{6a} and R^{6c}, or R^{6b} and R^{6c} may bond together to form a non-aromatic ring of 3 to 8 carbon atoms with the carbon atom to which they are attached. R^{7a} is hydrogen, or a straight, branched or cyclic C₁-C₁₅ monovalent hydrocarbon group, typically alkyl, R^{7b} is a straight, branched or cyclic C₁-C₁₅ monovalent hydrocarbon group, typically alkyl, or R^{7a} and R^{7b} may bond together to form a non-aromatic ring of 3 to 8 carbon atoms with the carbon atom to which they are attached. R^{8a}, R^{8b} and R^{8c} are each independently a straight, branched or cyclic C₁-C₁₅ monovalent fluorinated hydrocarbon group, typically fluoroalkyl, R^{9a} is a straight, branched or cyclic C₁-C₁₅ monovalent hydrocarbon or fluorinated hydrocarbon group, typically alkyl or fluoroalkyl, and k² is 0 or 1.

The polymeric surfactant to be added to the resist composition is improved in water repellency, water slip, alkaline solubility, and contact angle after development by incorporating recurring units of one or more type selected from formulae (2a) to (2j) in addition to the recurring units having formula (1a).

Examples of the straight, branched or cyclic C₁-C₁₅ alkyl groups of R^{4a}, R^{4b}, R^{5a}, R^{6a}, R^{6b}, R^{6c}, R^{7a}, R^{7b}, R^{8a}, and R^{9a} include methyl, ethyl, n-propyl, isopropyl, n-butyl, sec-butyl, tert-butyl, tert-amyl, n-pentyl, n-hexyl, n-heptyl, n-octyl, n-nonyl, n-decyl, cyclopentyl, cyclohexyl, cyclopentylmethyl, cyclopentylethyl, cyclopentylbutyl, cyclohexylmethyl, cyclohexylethyl, cyclohexylbutyl, and adamantyl. A pair of R^{4a} and R^{4b}, R^{6a} and R^{6b}, R^{6a} and R^{6c}, R^{6b} and R^{6c}, or R^{7a} and R^{7b} may bond together to form a C₃-C₈ non-aromatic ring with the carbon atom to which they are attached. In the event of cyclization, each R is alkylene, examples of which are the foregoing alkyl groups with one hydrogen eliminated therefrom, and exemplary rings are cyclopentyl and cyclohexyl.

R^{5a}, R^{8a}, and R^{9a} stand for straight, branched or cyclic C₁-C₁₅ monovalent fluorinated hydrocarbon groups, specifically fluoroalkyl groups which are typically substituted forms of the foregoing alkyl groups in which some or all hydrogen atoms are substituted by fluorine atoms. Examples include, but are not limited to, trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, 2-(perfluorodecyl)ethyl, and 3,3,4,4,5,5,6,6,6-nonafluorohexyl. Examples of the straight, branched or cyclic C₁-C₁₀ fluoroalkyl group represented by R^{8a} include trifluoromethyl, 2,2,2-trifluoroethyl, 3,3,3-trifluoro-1-propyl, 3,3,3-trifluoro-2-propyl, 2,2,3,3-tetrafluoropropyl, 1,1,1,3,3,3-hexafluoroisopropyl, 2,2,3,3,4,4,4-heptafluorobutyl, 2,2,3,3,4,4,5,5-octafluoropentyl, 2,2,3,3,4,4,5,5,6,6,7,7-dodecafluoroheptyl, 2-(perfluorobutyl)ethyl, 2-(perfluorohexyl)ethyl, 2-(perfluorooctyl)ethyl, and 3,3,4,4,5,5,6,6,6-nonafluorohexyl.

The acid labile group represented by R^{5a} may be selected from such groups as described in conjunction with component (C) and represented by XA in the recurring unit having formula (3). Notably, the groups of formula (L2) wherein y=0 are excluded herein.

Illustrative examples of the recurring units having formulae (2a) to (2j) are given below, but not limited thereto.

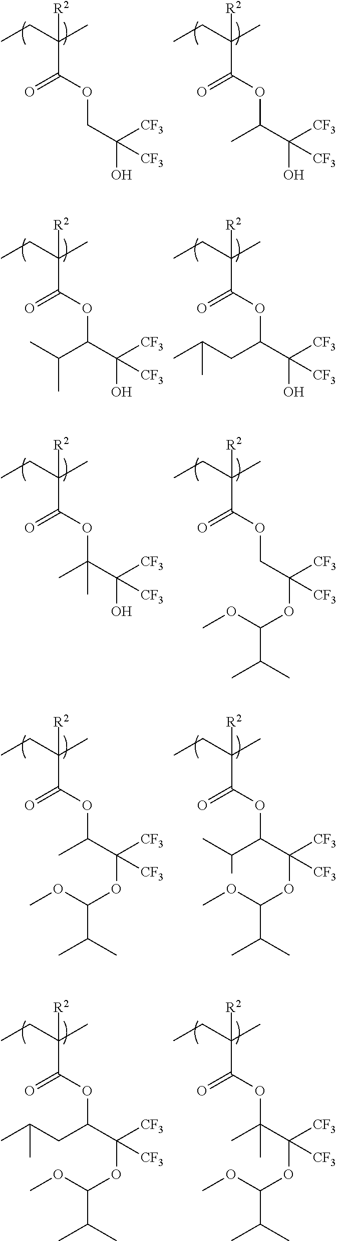

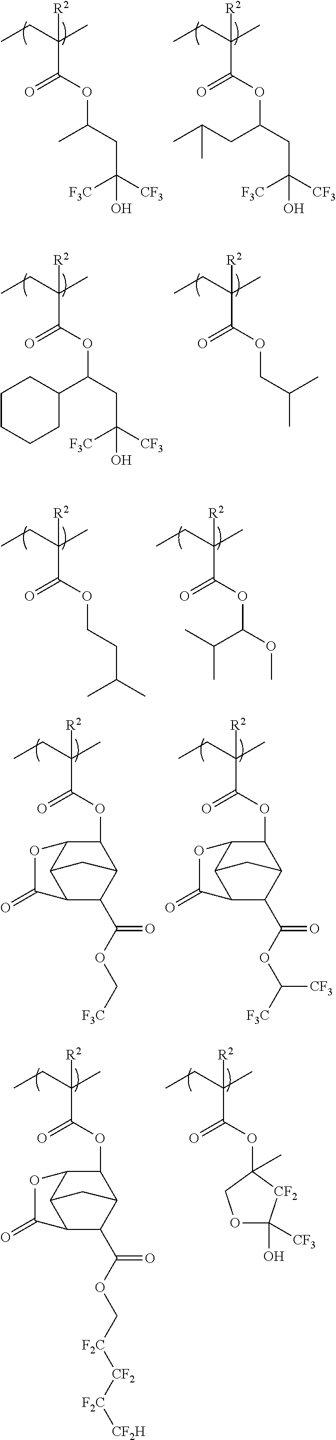

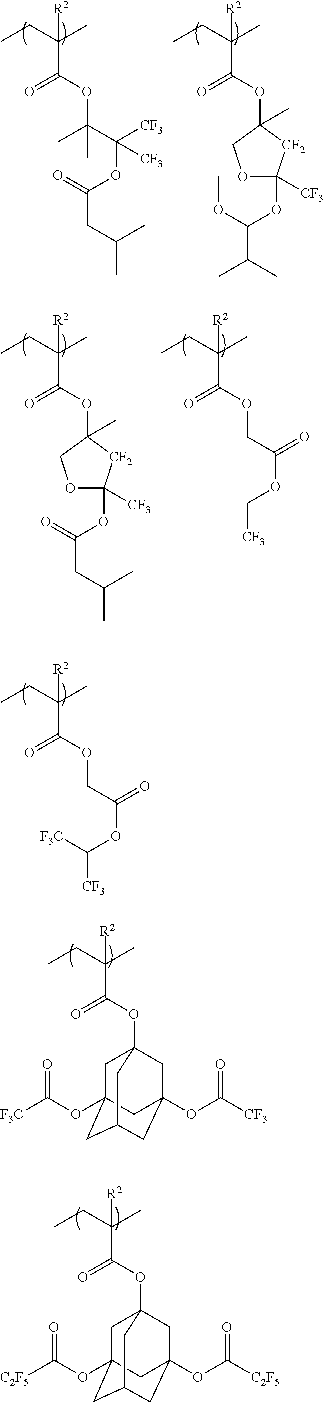

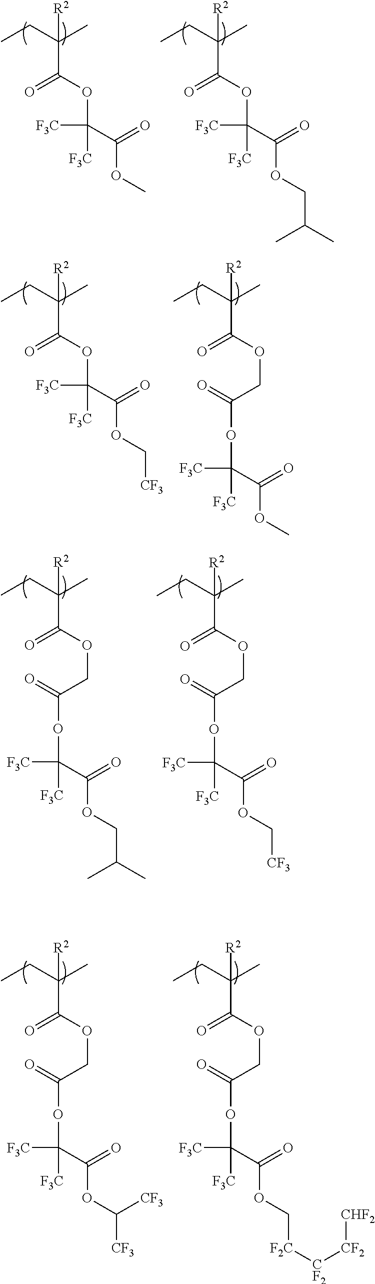

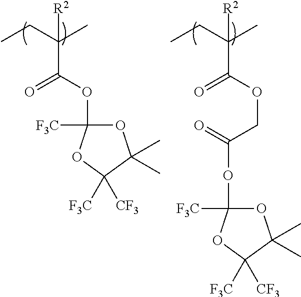

Note that R² is as defined above.

Although the polymeric surfactant comprising recurring units of formula (1a) in combination with recurring units of formulae (2a) to (2j) exerts satisfactory performance, recurring units of one or multiple types selected from formulae (3a) to (3e), (4a) to (4e), (5a) to (5c), and (6a) to (6c) may be further incorporated therein for the purposes of imparting further water repellency and water slip, and controlling alkaline solubility and developer affinity.

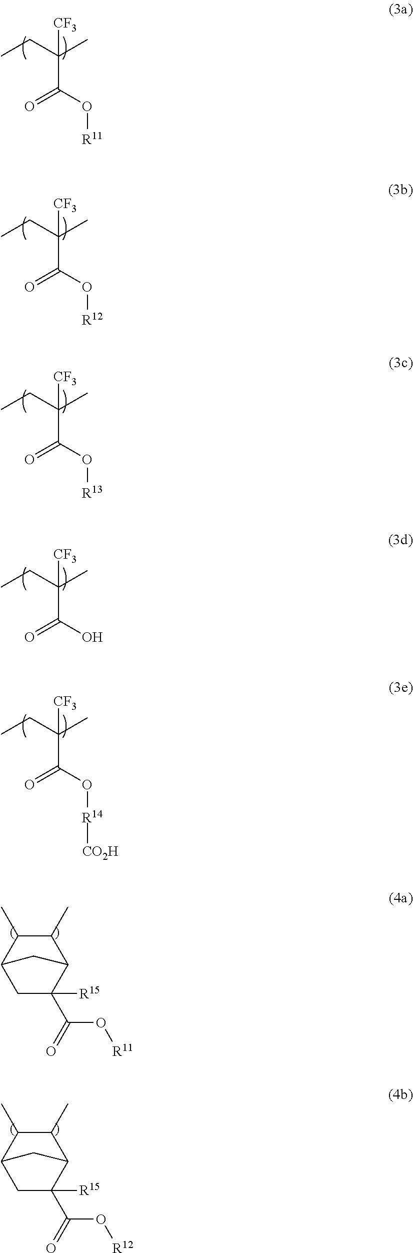

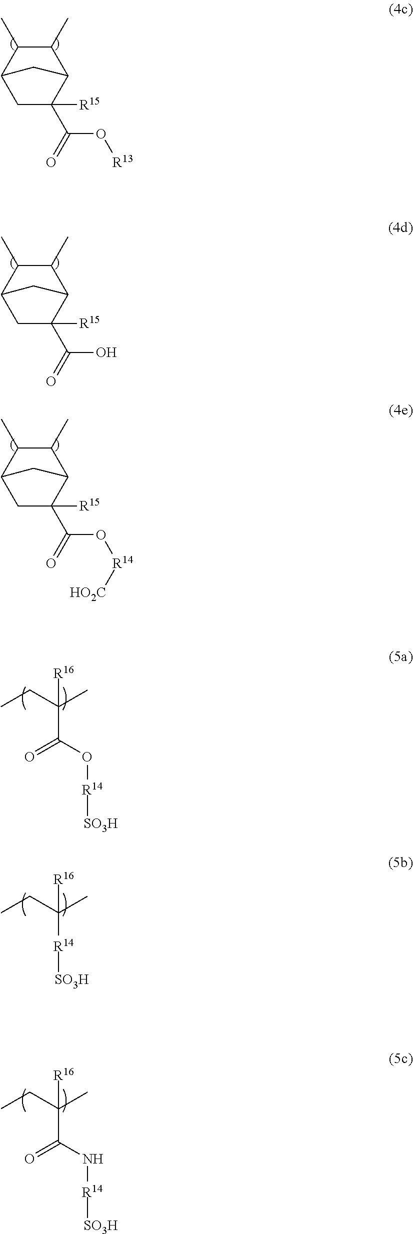

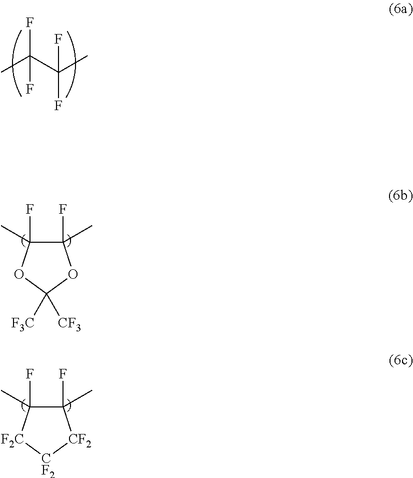

Herein R¹¹ is a C₁-C₁₅ monovalent hydrocarbon or fluorinated hydrocarbon group, typically alkyl or fluoroalkyl, R¹² is an adhesive group, R¹³ is an acid labile group, R¹⁴ is a single bond or divalent C₁-C₁₅ organic group, and R¹⁵ and R¹⁶ each are hydrogen, methyl or trifluoromethyl.

Examples of the C₁-C₁₅ monovalent hydrocarbon and fluorinated hydrocarbon groups represented by R¹¹ are the same as R^{5a}, R^{8a} and R^{9a}.

The adhesive group represented by R¹² may be selected from a variety of such groups, typically those groups shown below.

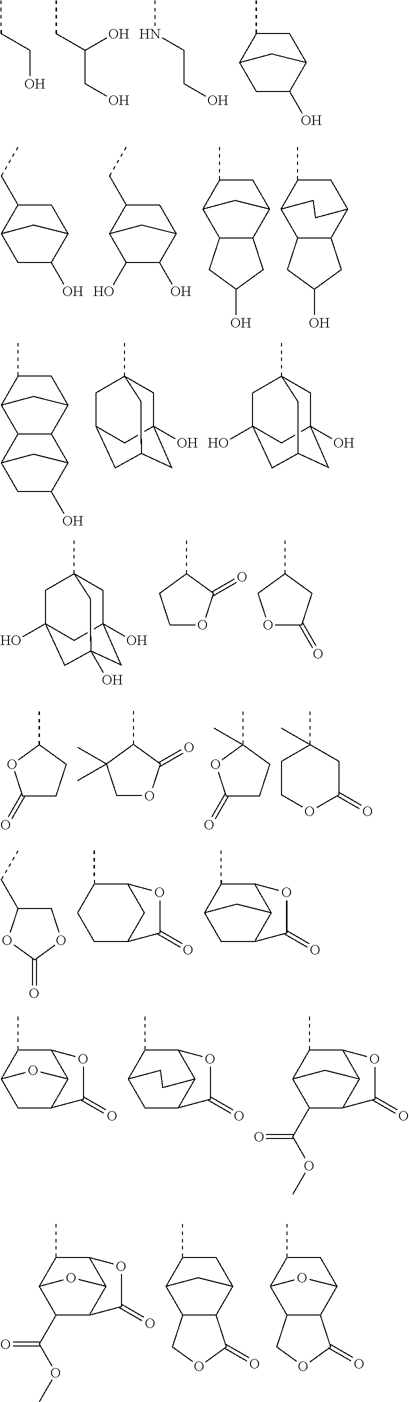

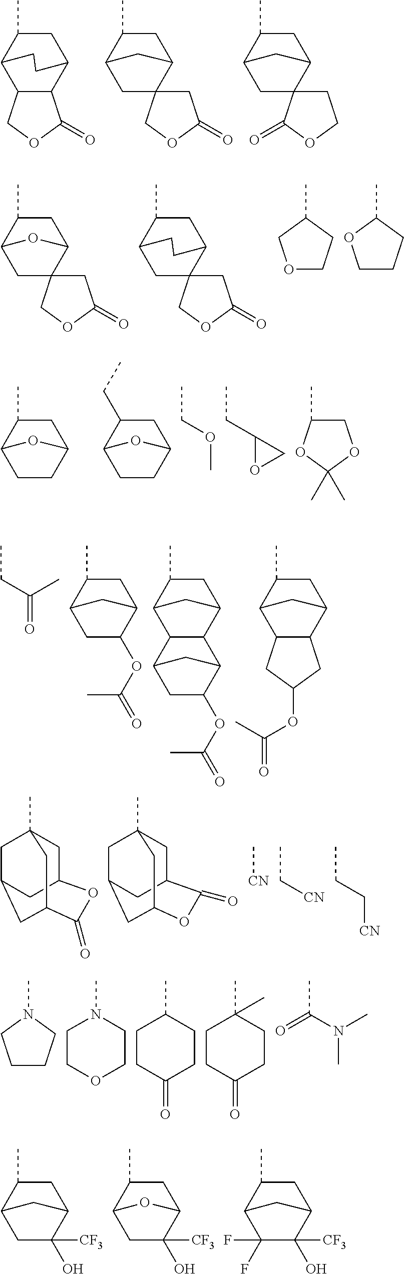

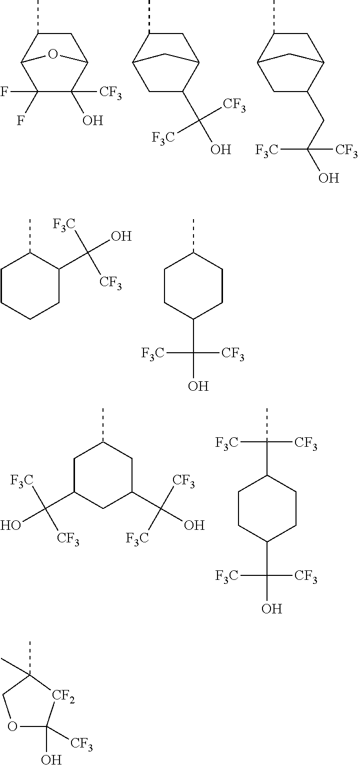

The acid labile group represented by R¹³ may be selected from such groups as described in conjunction with component (C) and represented by XA in the recurring unit having formula (3).

Suitable divalent C₁-C₁₅ organic groups represented by R¹⁴ include the above-exemplified monovalent hydrocarbon groups, typically alkyl groups, with one hydrogen atom eliminated (e.g., methylene and ethylene). Also useful are groups of the following formulae.

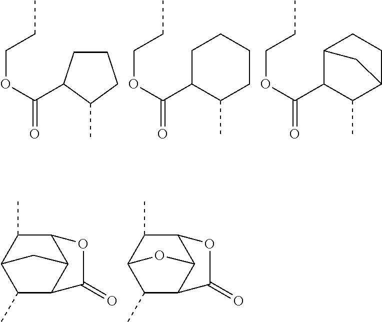

The polymeric surfactant is preferably constructed such that a proportion of respective recurring units derived from different monomers may fall in the following range (mol %) though it is not limited thereto. In the polymeric surfactant wherein U1 stands for a total molar number of a monomer corresponding to units of formula (1a), U2 stands for a total molar number of monomers corresponding to units of formulae (2a) to (2j), and U3 stands for a total molar number of monomers corresponding to units of formulae (3a) to (3e), (4a) to (4e), (5a) to (5c), and (6a) to (6c), with the proviso that U1+U2+U3=U (=100 mol %), values of U1, U2, and U3 are preferably determined so as to meet:

0<U1/U<1, more preferably 0.1≦U1/U≦0.8, even more preferably 0.1≦U1/U≦0.7,

0≦U2/U<1, more preferably 0.2≦U2/U≦0.9, even more preferably 0.3≦U2/U≦0.9, and

0≦U3/U<1, more preferably 0≦U3/U≦0.4, even more preferably 0≦U3/U≦0.2.

The polymeric surfactant used herein may be obtained from copolymerization reaction using a compound having the general formula (2) as a first monomer and polymerizable double bond-bearing compounds as second and subsequent monomers.

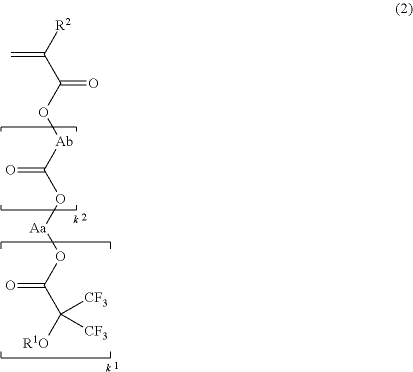

Herein R¹, R², Aa, Ab, k¹, and k² are as defined above.

The copolymerization reaction to produce the polymeric surfactant may be performed in various modes, preferably radical polymerization, anionic polymerization or coordination polymerization.

For radical polymerization, preferred reaction conditions include (a) a solvent selected from among hydrocarbons such as benzene, ethers such as tetrahydrofuran, alcohols such as ethanol, and ketones such as methyl isobutyl ketone, (b) a polymerization initiator selected from azo compounds such as 2,2′-azobisisobutyronitrile and peroxides such as benzoyl peroxide and lauroyl peroxide, (c) a temperature of about 0° C. to about 100° C., and (d) a time of about 0.5 hour to about 48 hours. Reaction conditions outside the described range may be employed if desired.

The polymeric surfactant comprising recurring units having formula (1a) preferably has a weight average molecular weight (Mw) of 1,000 to 50,000, more preferably 2,000 to 20,000, as measured by GPC versus polystyrene standards. Outside the range, there may result insufficient surface modification and development defects.

An appropriate amount of the polymeric surfactant added is 0.001 to 20 parts, more preferably 0.01 to 10 parts by weight per 100 parts by weight of the base resin.

While the polymeric surfactant comprises recurring units having formula (1a), each unit contains a plurality of fluorine atoms. When this polymer is added to a resist composition, the polymer itself functions as surfactant so that the polymer may segregate on the resist film surface at the same time as film formation.

In general, a fluorinated polymer exhibits excellent properties of water repellency and water slip. On use of this polymer as a resist additive, a resist film surface having good water repellency and water slip is available at the same time as film formation. That is, an effect equivalent to the use of a resist protective film is expectable. This approach is advantageous from the cost aspect too because the steps of forming and removing a resist protective film are unnecessary.

Now it is described how to prepare the polymer (or polymeric surfactant) comprising recurring units having formula (1a). The polymer comprising recurring units having formula (1a) is prepared using a fluorinated monomer having formula (2). As seen from the reaction scheme shown below, a fluorinated monomer having formula (2) wherein k²=0 may be prepared via step i); and a fluorinated monomer having formula (2) wherein k²=1 may be prepared via step ii) or steps iii) and iv). Notably the synthesis route is not limited thereto.

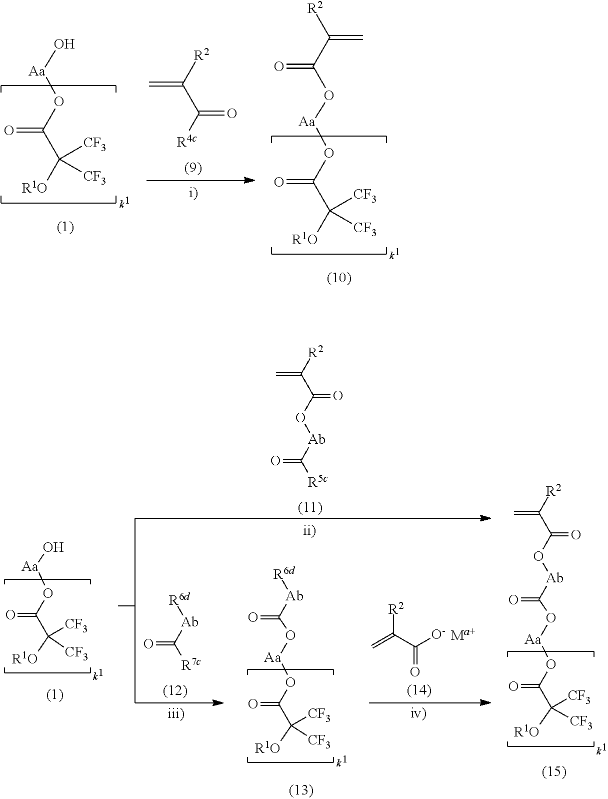

In these formulae, R¹, R², Aa, Ab, k¹ and k² are as defined above. R^{4c} is halogen, hydroxyl or —OR⁸ wherein R⁸ is methyl, ethyl or a group of the following formula (16).

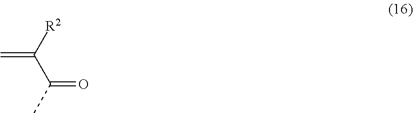

R^{5c} is halogen, hydroxyl or —OR⁹ wherein R⁹ is methyl, ethyl or a group of the following formula (17).

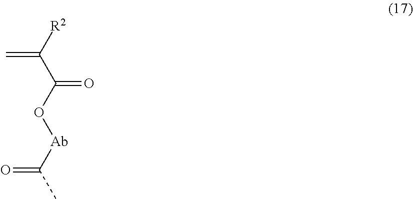

R^{6d} is halogen. R^{7c} is halogen, hydroxyl or —OR¹⁰ wherein R¹⁰ is methyl, ethyl or a group of the following formula (18).

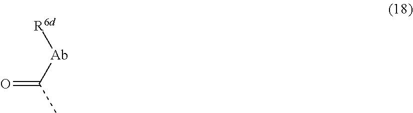

M^{a} is Li, Na, K, Mg_{1/2}, Ca_{1/2} or substituted or unsubstituted ammonium.

Step i) is reaction of an alcohol compound (1) with an esterifying agent (9) to form a monomer (10), that is, fluorinated monomer (2).

The reaction may readily run by a well-known procedure. The preferred esterifying agent (9) is an acid chloride of formula (9) wherein R^{4c} is chlorine or a carboxylic anhydride of formula (9) wherein R^{4c} is —OR⁸ and R⁸ has the following formula (16).

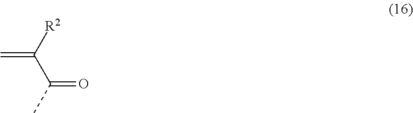

When an acid chloride such as methacrylic acid chloride is used as the esterifying agent (9), the reaction may be conducted in a solventless system or in a solvent (e.g., methylene chloride, acetonitrile, toluene or hexane) by adding alcohol compound (1), acid chloride, and a base (e.g., triethylamine, pyridine or 4-dimethylaminopyridine) in sequence or at the same time, and optional cooling or heating. When a carboxylic anhydride such as methacrylic anhydride is used as the esterifying agent (9), the reaction may be conducted in a solvent (e.g., toluene or hexane) by heating alcohol compound (1) and carboxylic anhydride in the presence of an acid catalyst, and optionally removing the resulting water out of the system. Suitable acid catalysts include mineral acids such as hydrochloric acid, sulfuric acid, nitric acid, and perchloric acid, and organic acids such as methanesulfonic acid, trifluoromethanesulfonic acid, p-toluenesulfonic acid and benzenesulfonic acid.

Step ii) is reaction of an alcohol compound (1) with an esterifying agent (11) to form a monomer (15), that is, fluorinated monomer (2).

The reaction may readily run by a well-known procedure. The preferred esterifying agent (11) is an acid chloride of formula (11) wherein R^{5c} is chlorine or a carboxylic anhydride of formula (11) wherein R^{5c} is —OR⁹ and R⁹ has the following formula (17).

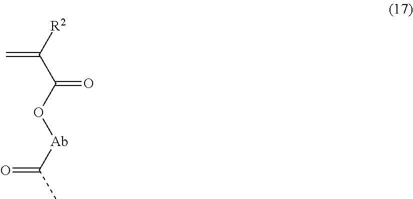

When an acid chloride such as methacryloyloxyacetic acid chloride is used as the esterifying agent (11), the reaction may be conducted in a solventless system or in a solvent (e.g., methylene chloride, acetonitrile, toluene or hexane) by adding alcohol compound (1), acid chloride, and a base (e.g., triethylamine, pyridine or 4-dimethylaminopyridine) in sequence or at the same time, and optional cooling or heating. When a carboxylic anhydride such as methacryloyloxyacetic anhydride is used as the esterifying agent (11), the reaction may be conducted in a solvent (e.g., toluene or hexane) by heating alcohol compound (1) and carboxylic anhydride in the presence of an acid catalyst, and optionally removing the resulting water out of the system. Suitable acid catalysts include mineral acids such as hydrochloric acid, sulfuric acid, nitric acid, and perchloric acid, and organic acids such as methanesulfonic acid, trifluoromethanesulfonic acid, p-toluenesulfonic acid and benzenesulfonic acid.

Step iii) is reaction of an alcohol compound (1) with an esterifying agent (12) to form a halo-ester compound (13).

The reaction may readily run by a well-known procedure. The preferred esterifying agent (12) is an acid chloride of formula (12) wherein R^{7c} is chlorine or a carboxylic acid of formula (12) wherein R^{7c} is hydroxyl. When an acid chloride such as 2-chloroacetic acid chloride or 4-chlorobutyric acid chloride is used as the esterifying agent (12), the reaction may be conducted in a solventless system or in a solvent (e.g., methylene chloride, toluene, hexane, diethyl ether, tetrahydrofuran or acetonitrile) by adding alcohol compound (1), acid chloride, and a base (e.g., triethylamine, pyridine or 4-dimethylaminopyridine) in sequence or at the same time, and optional cooling or heating. When a carboxylic acid such as 4-chloroacetic acid or 4-chlorobutyric acid is used as the esterifying agent (12), the reaction may be conducted in a solvent (e.g., toluene or hexane) by heating alcohol compound (1) and carboxylic acid in the presence of an acid catalyst, and optionally removing the resulting water out of the system. Suitable acid catalysts include mineral acids such as hydrochloric acid, sulfuric acid, nitric acid, and perchloric acid, and organic acids such as p-toluenesulfonic acid and benzenesulfonic acid.

Step iv) is reaction of halo-ester compound (13) with a carboxylic acid salt (14) to form a monomer (15), that is, fluorinated monomer (2).

The reaction may readily run by a well-known procedure. As the carboxylic acid salt (14), commercially available carboxylic acid salts, typically metal salts of carboxylic acids may be used as such. Alternatively, the carboxylic acid salt may be prepared within the reaction system using a corresponding carboxylic acid such as methacrylic acid or acrylic acid and a base. An appropriate amount of the carboxylic acid salt (14) used is 0.5 to 10 moles, more preferably 1.0 to 3.0 moles per mole of halo-ester compound (13). With less than 0.5 mole of the carboxylic acid salt, a larger fraction of the reactant may be left unreacted, leading to a substantial drop of yield. More than 10 moles may be uneconomical because of an increase of reactant cost and a lowering of pot yield. When the carboxylic acid salt is prepared in situ using a corresponding carboxylic acid and a base, suitable bases which can be used include amines such as ammonia, triethylamine, pyridine, lutidine, collidine, and N,N-dimethylaniline; hydroxides such as sodium hydroxide, potassium hydroxide, and tetramethylammonium hydroxide; carbonates such as potassium carbonate and sodium hydrogencarbonate; metals such as sodium; metal hydrides such as sodium hydride; metal alkoxides such as sodium methoxide and potassium t-butoxide; organometallic compounds such as butyl lithium and ethylmagnesium bromide; and metal amides such as lithium diisopropylamide, which may be used alone or in admixture. An appropriate amount of the base used is 0.2 to 10 moles, more preferably 0.5 to 2.0 moles per mole of the carboxylic acid. Less than 0.2 mole of the base may lead to an economic waste because a larger fraction of the carboxylic acid is left in vain. More than 10 moles of the base may allow for more side reactions, leading to a substantial drop of yield.

Examples of the solvent used in step iv) include hydrocarbons such as toluene, xylene, hexane, and heptane; chlorinated solvents such as methylene chloride, chloroform and dichloroethane; ethers such as diethyl ether, tetrahydrofuran and dibutyl ether; ketones such as acetone and 2-butanone; esters such as ethyl acetate and butyl acetate; nitriles such as acetonitrile; alcohols such as methanol and ethanol; aprotic polar solvents such as N,N-dimethylformamide, N,N-dimethylacetamide and dimethyl sulfoxide; and water, which may be used alone or in admixture. To the reaction system, a phase transfer catalyst such as tetrabutylammonium hydrogensulfate may be added. An appropriate amount of the phase transfer catalyst, if used, is 0.0001 to 1.0 mole, more preferably 0.001 to 0.5 mole per mole of halo-ester compound (13). Less than 0.0001 mole of the catalyst may fail to achieve an addition effect whereas more than 1.0 mole of the catalyst may be uneconomical because of an increased catalyst cost.

The temperature for the esterifying reaction preferably ranges from −70° C. to approximately the boiling point of the solvent used. An appropriate temperature may be selected in accordance with other reaction conditions, although it is most often in the range of 0° C. to approximately the boiling point of the solvent used. Since noticeable side reactions occur at higher temperatures, it is important for gaining higher yields that the reaction run at a temperature which is low, but enough to ensure a practically acceptable reaction rate. The reaction time is determined as appropriate by monitoring the reaction process by thin-layer chromatography (TLC) or gas chromatography (GC) because it is desirable from the yield aspect to drive the reaction to completion. Usually the reaction time is about 30 minutes to about 40 hours. The monomer (15), that is, desired fluorinated monomer (2) may be obtained from the reaction mixture by ordinary aqueous work-up. If necessary, the monomer may be purified by standard techniques like distillation, recrystallization and chromatography.

Understandably, the fluorinated alcohol compound (1) may be prepared according to the reaction scheme shown below although the synthesis route is not limited thereto.

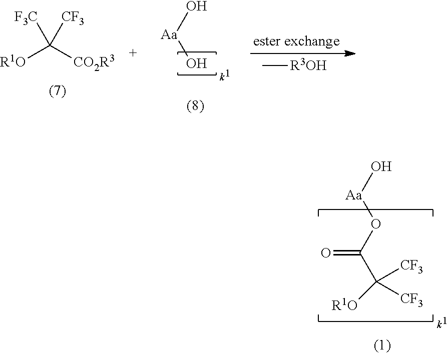

Herein R¹, Aa and k¹ are as defined above, and R³ is hydrogen or a C₁-C₆ straight, branched or cyclic monovalent hydrocarbon group.

The fluorinated alcohol compound (1) may be synthesized by ester exchange reaction between a fluorine compound (7) and a corresponding polyhydric alcohol compound (8). It is noted that the fluorine compound (7), which is a 3,3,3-trifluoro-2-hydroxy-2-(trifluoromethyl)propionic acid derivative, may be obtained from octafluoroisobutylene which is one of byproducts formed during synthesis of hexafluoropropene or the like. Since the starting reactant is available as a byproduct of a commercially useful product, it is a fluorine compound supplied in plenty and at relatively low cost.

Although the reaction may be carried out in a solventless system, a solvent may be used in an auxiliary manner. Suitable solvents include ethers such as tetrahydrofuran, diethyl ether, di-n-butyl ether and 1,4-dioxane, and hydrocarbons such as n-hexane, n-heptane, benzene, toluene, xylene and cumene, which may be used alone or in admixture. Suitable catalysts include metal alkoxides such as sodium methoxide, sodium ethoxide, potassium t-butoxide, magnesium ethoxide, titanium(IV) methoxide, titanium(IV) ethoxide, and titanium(IV) isopropoxide, organic amines such as triethylamine, N,N-dimethylaminopyridine, and 1,8-diazabicyclo[5.4.0]-7-undecene, and inorganic bases such as sodium hydroxide, potassium carbonate and sodium carbonate, which may be used alone or in admixture. An appropriate amount of the catalyst used is 0.001 to 5.0 moles, more preferably 0.001 to 0.1 mole per mole of fluorine compound (7). Although the reaction temperature varies with other reaction conditions, the preferred temperature is 50 to 200° C. The reaction may be carried out while distilling off the concomitantly formed R³OH. The reaction time is determined as appropriate by monitoring the reaction process by silica gel thin-layer chromatography (TLC) or gas chromatography (GC) because it is desirable from the yield aspect to drive the reaction to completion. Usually the reaction time is about 0.5 to 20 hours. The fluorinated alcohol compound (1) may be obtained from the reaction mixture by ordinary aqueous work-up. If necessary, the compound may be purified by standard techniques like distillation, recrystallization and chromatography.

### Process

A further embodiment of the invention is a pattern forming process using the resist composition defined above. A pattern may be formed from the resist composition using any well-known lithography process. The preferred process includes at least the steps of forming a resist film on a substrate, exposing it to high-energy radiation, and developing it in a developer.

The resist composition is applied onto a substrate for integrated circuit fabrication (e.g., Si, SiO₂, SiN, SiON, TiN, WSi, BPSG, SOG or organic antireflective coating) by a suitable coating technique such as spin coating. The coating is prebaked on a hot plate at a temperature of 60 to 150° C. for 1 to 10 minutes, preferably 80 to 140° C. for 1 to 5 minutes, to form a resist film of 0.05 to 2.0 μm thick. The resist film is then exposed by the ArF immersion lithography. A mask having the desired pattern is placed over the resist film, a liquid, typically water, is interposed between the mask and the resist film, and the resist film is exposed to ArF excimer laser radiation in a dose of 1 to 200 mJ/cm², and preferably 10 to 100 mJ/cm². Prior to exposure, a protective film which is insoluble in water may be formed on the resist film, if desired.

After exposure, the resist film is baked (PEB) on a hot plate at 60 to 150° C. for 1 to 5 minutes, and preferably at 80 to 140° C. for 1 to 3 minutes. This is followed by development in a developer which is an alkaline aqueous solution, typically an aqueous solution of 0.1 to 5 wt %, more typically 2 to 3 wt % of tetramethylammonium hydroxide (TMAH). Development may be carried out by a conventional method such as dip, puddle, or spray development for 0.1 to 3 minutes, and preferably 0.5 to 2 minutes. These steps result in the formation of the desired pattern on the substrate.

The water-insoluble protective film which is used in the immersion lithography is to prevent any components from being leached out of the resist film and to improve water slippage at the film surface and is generally divided into two types. The first type is an organic solvent-strippable protective film which must be stripped, prior to alkaline development, with an organic solvent in which the resist film is not dissolvable. The second type is an alkali-soluble protective film which is soluble in an alkaline developer so that it can be removed simultaneously with the removal of solubilized regions of the resist film. The protective film of the second type is preferably of a material comprising a polymer having a 1,1,1,3,3,3-hexafluoro-2-propanol residue (which is insoluble in water and soluble in an alkaline developer) as a base in an alcohol solvent of at least 4 carbon atoms, an ether solvent of 8 to 12 carbon atoms or a mixture thereof. Alternatively, the aforementioned surfactant which is insoluble in water and soluble in an alkaline developer may be dissolved in an alcohol solvent of at least 4 carbon atoms, an ether solvent of 8 to 12 carbon atoms or a mixture thereof to form a material from which the protective film of the second type is formed.

Any desired step may be added to the pattern forming process. For example, after a photoresist film is formed, a step of rinsing with pure water (post-soaking) may be introduced to extract the acid generator or the like from the film surface or wash away particles. After exposure, a step of rinsing (post-soaking) may be introduced to remove any water remaining on the film after exposure.

The technique enabling the ArF lithography to survive to the 32-nm node is a double patterning process. The double patterning process includes a trench process of processing an underlay to a 1:3 trench pattern by a first step of exposure and etching, shifting the position, and forming a 1:3 trench pattern by a second step of exposure for forming a 1:1 pattern; and a line process of processing a first underlay to a 1:3 isolated left pattern by a first step of exposure and etching, shifting the position, processing a second underlay formed below the first underlay by a second step of exposure through the 1:3 isolated left pattern, for forming a half-pitch 1:1 pattern.

### EXAMPLE

Examples of the invention are given below by way of illustration and not by way of limitation. Mw and Mn are weight and number average molecular weights, respectively, as measured by GPC versus polystyrene standards, and Mw/Mn is a polydispersity index. Me stands for methyl.

### Synthesis Example 1

Fluorinated monomers within the scope of the invention were synthesized in accordance with the formulation shown below.

### Synthesis Example 1-1

### Synthesis of Monomer 1

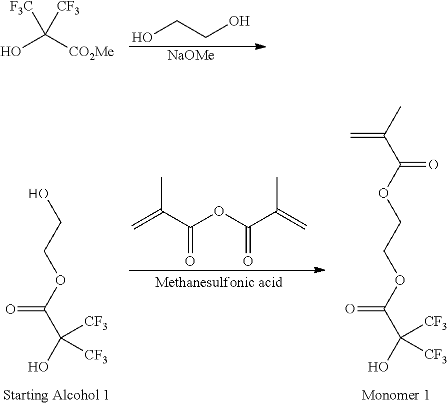

### Synthesis Example 1-1-1

### Synthesis of Starting Alcohol 1

A flask equipped with a distillation column was charged with 20.0 g of methyl 3,3,3-trifluoro-2-hydroxy-2-(trifluoromethyl)propionate, 16.5 g of ethylene glycol, 50 mL of benzene, and 0.9 g of sodium methoxide (28 wt % methanol solution). In a nitrogen atmosphere, the mixture was heated under reflux for 6 hours while methanol formed during reaction was sequentially distilled off. This was followed by ordinary aqueous work-up, solvent removal by distillation, and purification by distillation, obtaining 17.2 g of Starting Alcohol 1 (yield 76%).

Boiling point: 64° C./170 Pa

### Synthesis Example 1-1-2

### Synthesis of Monomer 1

To 3.30 g of Starting Alcohol 1 were added 1.92 g of methacrylic anhydride, 10 mL of toluene, and 0.05 g of methanesulfonic acid. In a nitrogen atmosphere, the mixture was heated and stirred at 50° C. for 10 hours. This was followed by ordinary aqueous work-up, solvent removal by distillation, and purification by distillation, obtaining 3.40 g of Monomer 1 (yield 81%).

Boiling point: 51° C./17 Pa

IR (thin film):
ν=3392, 2967, 1766, 1722, 1639, 1456, 1375, 1317, 1299, 1241, 1162, 1056, 1025, 1010, 977 cm⁻¹

¹H-NMR (600 MHz in DMSO-d₆):
δ=1.84 (3H, m), 4.34 (2H, m), 4.62 (2H, m), 5.69 (1H, m), 5.99 (1H, m), 9.23 (1H, s) ppm

¹⁹F-NMR (565 MHz in DMSO-d₆): δ=−75.03 (6F, s) ppm

### Synthesis Example 1-2

### Synthesis of Monomer 2

Monomer 2 was synthesized by the same procedure as Synthesis Example 1-1-2 aside from using acrylic anhydride instead of methacrylic anhydride. Two step yield 58%.

### Synthesis Example 1-3

### Synthesis of Monomer 3

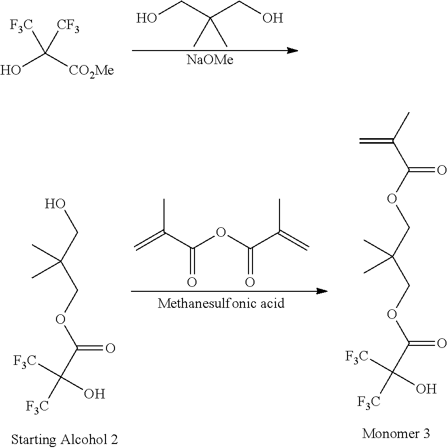

### Synthesis Example 1-3-1

### Synthesis of Starting Alcohol 2

Starting Alcohol 2 was synthesized by the same procedure as Synthesis Example 1-1-1 aside from using neopentyl glycol instead of ethylene glycol. Yield 72%.

IR (thin film):
ν=3546, 3515, 3164, 2978, 2950, 2889, 1764, 1480, 1379, 1328, 1256, 1225, 1162, 1040, 1014, 998, 978 cm⁻¹

¹H-NMR (600 MHz in DMSO-d₆):
δ=0.84 (6H, s), 3.17 (2H, s), 4.10 (2H, s), 4.70 (1H, s), 9.11 (1H, s) ppm

¹⁹F-NMR (565 MHz in DMSO-d₆): δ=−75.06 (6F, s) ppm

### Synthesis Example 1-3-2

### Synthesis of Monomer 3

Monomer 3 was synthesized by the same procedure as Synthesis Example 1-1-2 aside from using Starting Alcohol 2 instead of Starting Alcohol 1. Yield 92%.

Boiling point: 69° C./12 Pa

IR (thin film):
ν=3460, 3385, 2973, 1765, 1716, 1639, 1477, 1457, 1376, 1322, 1241, 1225, 1162, 1012, 977, 946 cm⁻¹

¹H-NMR (600 MHz in DMSO-d₆):
δ=0.95 (6H, s), 1.86 (3H, s), 3.89 (2H, s), 4.19 (2H, s), 5.67 (1H, m), 6.03 (1H, m), 9.21 (1H, s) Ppm

¹⁹F-NMR (565 MHz in DMSO-d₆): δ=−75.14 (6F, s) ppm

### Synthesis Example 1-4

### Synthesis of Monomer 4

Monomer 4 was synthesized by the same procedure as Synthesis Example 1-1-2 aside from using Starting Alcohol 2 instead of Starting Alcohol 1 and acrylic anhydride instead of methacrylic anhydride. Two-step yield 59%.

### Synthesis Example 1-5

### Synthesis of Monomer 5

Monomer 5 was synthesized by the same procedure as Synthesis Example 1-1-2 aside from using Starting Alcohol 2 instead of Starting Alcohol 1 and a-trifluoromethylacrylic anhydride instead of methacrylic anhydride. Two-step yield 48%.

### Synthesis Example 1-6

### Synthesis of Monomer 6

Monomer 6 was synthesized by the same procedure as Synthesis Examples 1-1-1 and 1-1-2 aside from using 4-methylpentane-1,3-diol instead of ethylene glycol. Two-step yield 38%.

Boiling point: 85-86° C./28 Pa

IR (thin film):
ν=3459, 2972, 1760, 1716, 1638, 1469, 1373, 1324, 1241, 1224, 1009, 977 cm⁻¹

¹H-NMR (600 MHz in DMSO-d₆):
δ=0.87 (6H, t), 1.87 (3H, s), 1.90-2.03 (3H, m), 4.24-4.31 (1H, m), 4.33-4.37 (1H, m), 4.78-4.82 (1H, m), 5.66 (1H, m), 6.03 (1H, m), 9.13 (1H, s) ppm

¹⁹F-NMR (565 MHz in DMSO-d₆): δ=−75.09 (6F, s) ppm

### Synthesis Example 1-7

### Synthesis of Monomer 7

Monomer 7 was synthesized by the same procedure as Synthesis Examples 1-1-1 and 1-1-2 aside from using 3-methylbutane-1,3-diol instead of ethylene glycol. Two-step yield 42%.

### Synthesis Example 1-8

### Synthesis of Monomer 8

Monomer 8 was synthesized by the same procedure as Synthesis Examples 1-1-1 and 1-1-2 aside from using neopentyl glycol instead of ethylene glycol. Two-step yield 37%.

IR (thin film):
ν=3461, 3334, 2995, 1763, 1711, 1640, 1474, 1317, 1303, 1246, 1220, 1163, 1016, 975 cm⁻¹

¹H-NMR (600 MHz in DMSO-d₆):
δ=1.04 (3H, s), 1.86 (3H, s), 4.02 (2H, s), 4.31 (4H, s), 5.69 (1H, m), 6.06 (1H, m), 9.30 (2H, s) ppm

¹⁹F-NMR (565 MHz in DMSO-d₆): δ=−75.28 (12F, s) ppm

### Synthesis Example 1-9

### Synthesis of Monomer 9

Monomer 9 was synthesized by the same procedure as Synthesis Examples 1-1-1 and 1-1-2 aside from using neopentyl glycol instead of ethylene glycol and acrylic anhydride instead of methacrylic anhydride. Two-step yield 34%.

### Synthesis Example 1-10

### Synthesis of Monomer 10

Monomer 10 was synthesized by the same procedure as Synthesis Examples 1-1-1 and 1-1-2 aside from using glycerol instead of ethylene glycol. Two-step yield 39%.

Boiling point: 97-98° C./11 Pa

IR (thin film):
ν=3469, 2972, 1766, 1722, 1638, 1455, 1382, 1314, 1227, 1161, 1013, 978 cm⁻¹

¹H-NMR (600 MHz in DMSO-d₆):
δ=1.81 (3H, s), 4.55-4.68 (4H, m), 5.39-5.43 (1H, m), 5.70 (1H, m), 5.97 (1H, m), 9.30 (2H, s) ppm

¹⁹F-NMR (565 MHz in DMSO-d₆): δ=−75.20 (12F, m) ppm

### Synthesis Example 1-11

### Synthesis of Monomer 11

Monomer 11 was synthesized by the same procedure as Synthesis Examples 1-1-1 and 1-1-2 aside from using 2,2-difluoro-4-methylbutane-1,3-diol instead of ethylene glycol. Two-step yield 31%.

### Synthesis Example 1-12

### Synthesis of Monomer 12

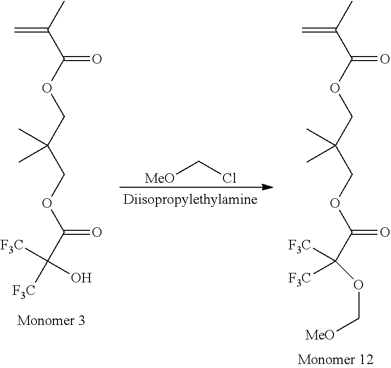

At a temperature below 20° C., 9.7 g of chloromethyl methyl ether was added dropwise to a mixture of 36.6 g of Monomer 3, 16.2 g of diisopropylethylamine, and 110 g of acetonitrile. The mixture was stirred at the temperature for 3 hours. This was followed by ordinary aqueous work-up, solvent removal by distillation, and purification by distillation, obtaining 39.8 g of Monomer 12 (yield 97%).

Boiling point: 78-79° C./12 Pa

IR (thin film):
ν=2970, 2935, 1766, 1723, 1639, 1477, 1402, 1374, 1296, 1257, 1229, 1161, 1074, 1028, 994, 935 cm⁻¹

¹H-NMR (600 MHz in DMSO-d₆):
δ=0.97 (6H, s), 1.86 (3H, s), 3.38 (3H, s), 3.89 (2H, s), 4.20 (2H, s), 5.04 (2H, s), 5.67 (1H, m), 6.03 (1H, m) ppm

¹⁹F-NMR (565 MHz in DMSO-d₆): δ=−72.49 (6F, s) ppm

### Synthesis Example 1-13

### Synthesis of Monomer 13

Monomer 13 was synthesized by the same procedure as Synthesis Example 1-12 aside from using isobutyric acid chloride instead of chloromethyl methyl ether. Yield 96%.

Boiling point: 90-91° C./11 Pa

IR (thin film):
ν=2979, 1773, 1724, 1639, 1472, 1401, 1374, 1260, 1235, 1163, 1117, 1086, 1041, 999, 943 cm⁻¹

¹H-NMR (600 MHz in DMSO-d₆):
δ=0.95 (6H, s), 1.14 (6H, d), 1.86 (3H, s), 2.86 (1H, sept), 3.85 (2H, s), 4.17 (2H, s), 5.67 (1H, m), 6.03 (1H, m) ppm

¹⁹F-NMR (565 MHz in DMSO-d₆): δ=−71.57 (6F, s) ppm

### Synthesis Example 1-14

### Synthesis of Monomer 14

Monomer 14 was synthesized by the same procedure as Synthesis Example 1-12 aside from using Monomer 8 instead of Monomer 3. Yield 96%.

Boiling point: 116-117° C./9 Pa

IR (thin film):
νν=2970, 2837, 1769, 1726, 1640, 1474, 1453, 1404, 1380, 1295, 1255, 1229, 1159, 1145, 1074, 1029, 994, 934 cm⁻¹

¹H-NMR (600 MHz in DMSO-d₆):
δ=1.05 (3H, s), 1.87 (3H, s), 3.38 (6H, s), 4.01 (2H, s), 4.31-4.36 (4H, m), 5.05 (4H, s), 5.70 (1H, m), 6.07 (1H, m) ppm

¹⁹F-NMR (565 MHz in DMSO-d₆): δ=−72.58 (12F, s) ppm

### Synthesis Example 1-15

### Synthesis of Monomer 15

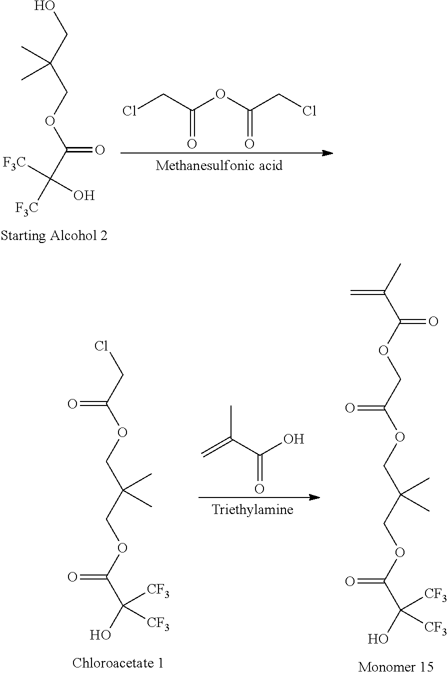

### Synthesis Example 1-15-1

### Synthesis of Chloroacetate 1

Chloroacetate 1 was synthesized by the same procedure as Synthesis Example 1-1-2 aside from using Starting Alcohol 2 instead of Starting Alcohol 1 and chloroacetic anhydride instead of methacrylic anhydride. Yield 88%.

### Synthesis Example 1-15-2

### Synthesis of Monomer 15

At a temperature below 25° C., a mixture of 137 g of triethylamine and 100 g of dimethylformamide was added dropwise to a mixture of 129 g of methacrylic acid, 139 g of Chloroacetate 1 (Synthesis Example 1-15-1), 22.0 g of sodium iodide and 400 g of dimethylformamide. The mixture was stirred at the temperature for 8 hours. Below 30° C., 300 g of 10 wt % hydrochloric acid was added. This was followed by ordinary work-up and vacuum distillation, obtaining 132 g of Monomer 15 (yield 84%).

Monomers 1 to 15 of the foregoing Synthesis Examples are identified below by their structural formulae.

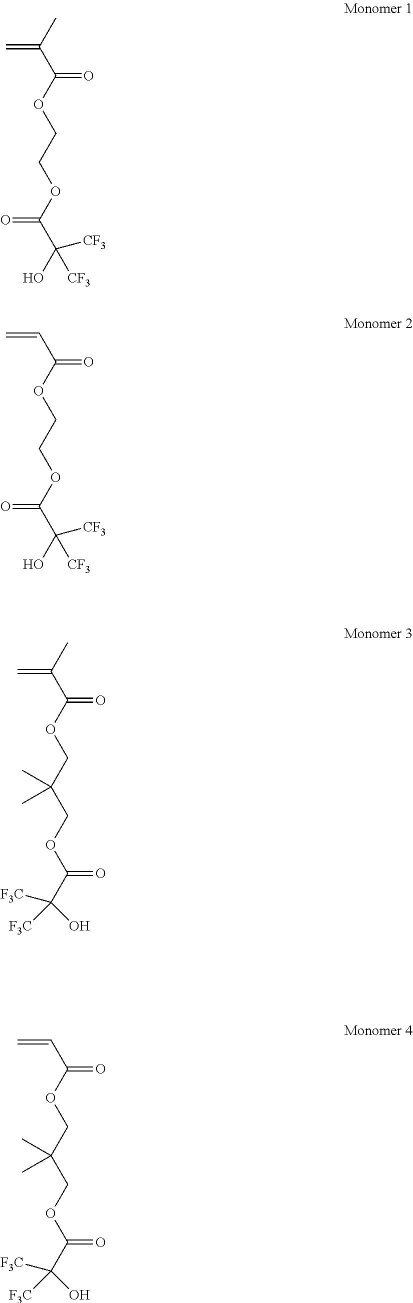 [SMILES: C=C(C)C(=O)OCCOC(=O)C(O)(C(F)(F)F)C(F)(F)F ; C=CC(=O)OCCOC(=O)C(O)(C(F)(F)F)C(F)(F)F ; C=C(C)C(=O)OCC(C)(C)COC(=O)C(O)(C(F)(F)F)C(F)(F)F ; C=CC(=O)OCC(C)(C)COC(=O)C(O)(C(F)(F)F)C(F)(F)F]

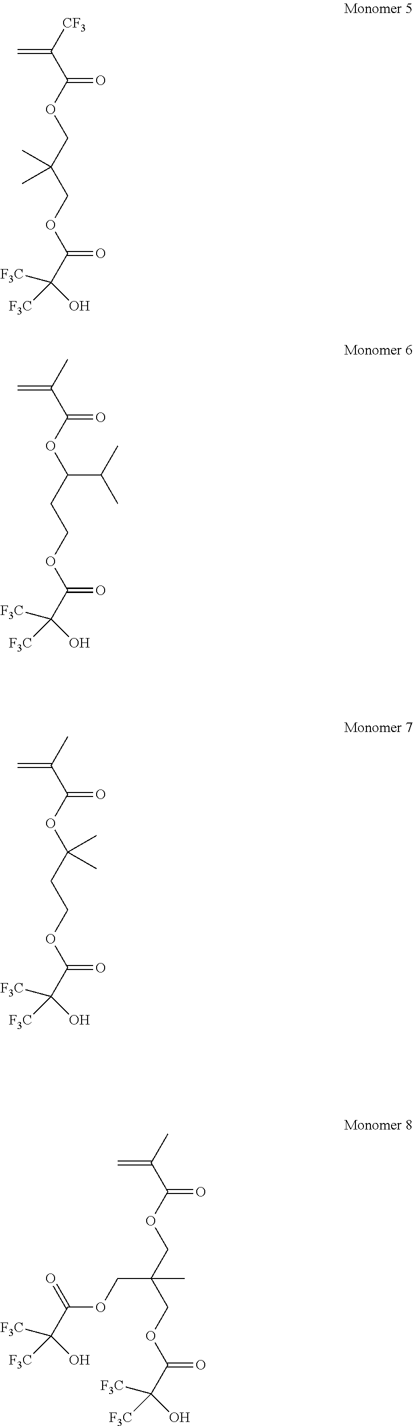 [SMILES: C=C(C(=O)OCC(C)(C)COC(=O)C(O)(C(F)(F)F)C(F)(F)F)C(F)(F)F ; C=C(C)C(=O)OC(CCOC(=O)C(O)(C(F)(F)F)C(F)(F)F)C(C)C ; C=C(C)C(=O)OC(C)(C)CCOC(=O)C(O)(C(F)(F)F)C(F)(F)F ; C=C(C)C(=O)OCC(C)(COC(=O)C(O)(C(F)(F)F)C(F)(F)F)COC(=O)C(O)(C(F)(F)F)C(F)(F)F]

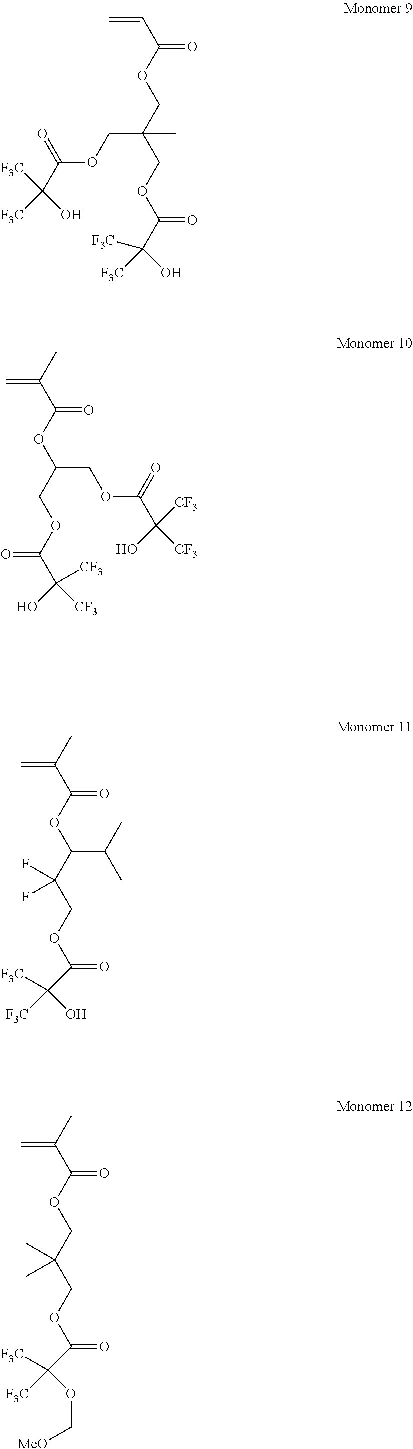 [SMILES: C=CC(=O)OCC(C)(COC(=O)C(O)(C(F)(F)F)C(F)(F)F)COC(=O)C(O)(C(F)(F)F)C(F)(F)F ; C=C(C)C(=O)OC(COC(=O)C(O)(C(F)(F)F)C(F)(F)F)COC(=O)C(O)(C(F)(F)F)C(F)(F)F ; C=C(C)C(=O)OC(C(C)C)C(F)(F)COC(=O)C(O)(C(F)(F)F)C(F)(F)F ; C=C(C)C(=O)OCC(C)(C)COC(=O)C(OCOC)(C(F)(F)F)C(F)(F)F]

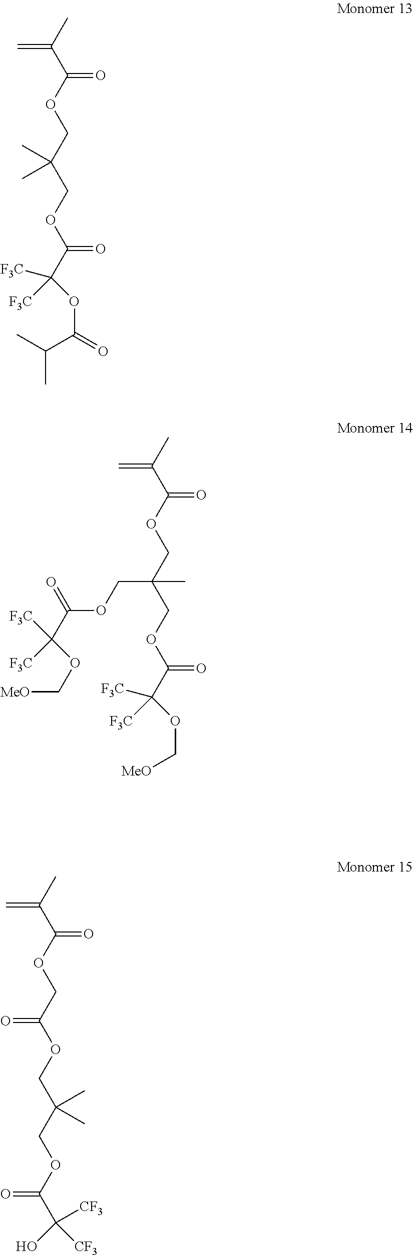 [SMILES: C=C(C)C(=O)OCC(C)(C)COC(=O)C(OC(=O)C(C)C)(C(F)(F)F)C(F)(F)F ; C=C(C)C(=O)OCC(C)(COC(=O)C(OCOC)(C(F)(F)F)C(F)(F)F)COC(=O)C(OCOC)(C(F)(F)F)C(F)(F)F ; C=C(C)C(=O)OCC(=O)OCC(C)(C)COC(=O)C(O)(C(F)(F)F)C(F)(F)F]

### Synthesis Example 2

Polymeric surfactants used in the invention were synthesized according to the following formulation.

### Synthesis Example 2-1

### Synthesis of Polymer 1

In a nitrogen atmosphere, a flask was charged with 15.0 g of ethylene glycol methacrylate [3,3,3-trifluoro-2-hydroxy-2-(trifluoromethyl)propionate], 0.53 g of dimethyl 2,2′-azobisisobutyrate, and 15.0 g of methyl ethyl ketone to form a monomer solution, which was kept at 20-25° C. In a nitrogen atmosphere, another flask was charged with 7.50 g of methyl ethyl ketone, which was heated at 80° C. with stirring. The monomer solution was added dropwise thereto over 4 hours. After the completion of dropwise addition, the polymerization solution was stirred for a further 2 hours while maintaining the temperature of 80° C. At the end of maturing, the solution was cooled to room temperature. The polymerization solution was transferred to an eggplant-shape flask and concentrated on an evaporator. Toluene was added to the concentrate so as to eventually form a 40 wt % solution of toluene/methyl ethyl ketone (mix ratio 9/1). The solution was added dropwise to 150 g of hexane whereupon a copolymer precipitated. The copolymer was collected by filtration, washed with 90 g of hexane, and separated as a white solid. The white solid was vacuum dried at 50° C. for 20 hours, yielding the target polymer, designated Polymer 1, in white powder solid form. Amount 12.7 g, yield 80%.

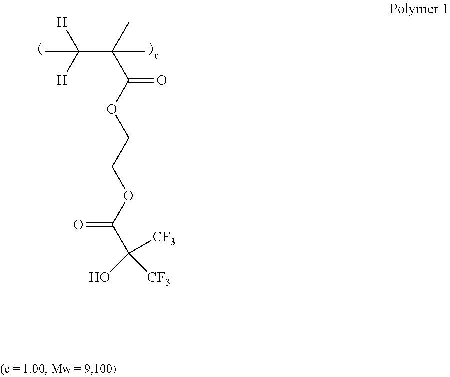 [SMILES: CCC(C)(C)C(=O)OCCOC(=O)C(O)(C(F)(F)F)C(F)(F)F]

### Synthesis Examples 2-2 to 2-22

### Synthesis of Polymers 2 to 22

Polymers 2 to 22 were synthesized as in Synthesis Example 2-1 aside from changing the amount and type of monomers. It is noted that the values of c, d, e and f are molar ratios of monomer units.

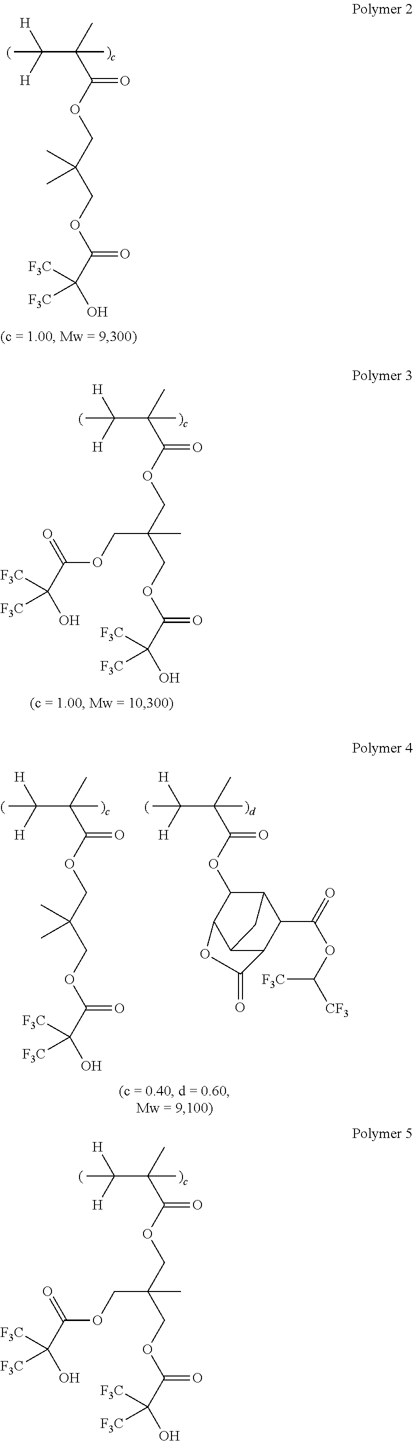

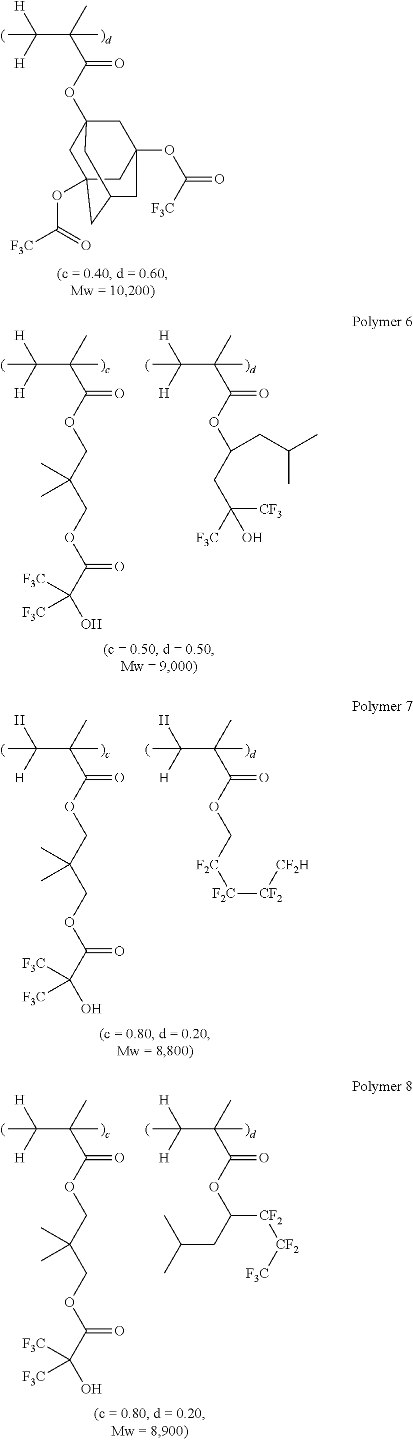

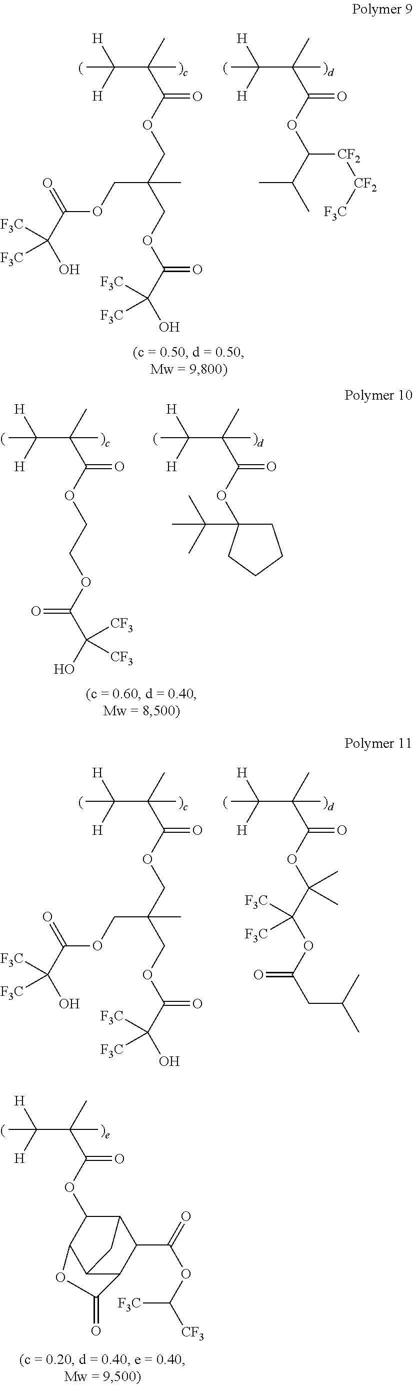

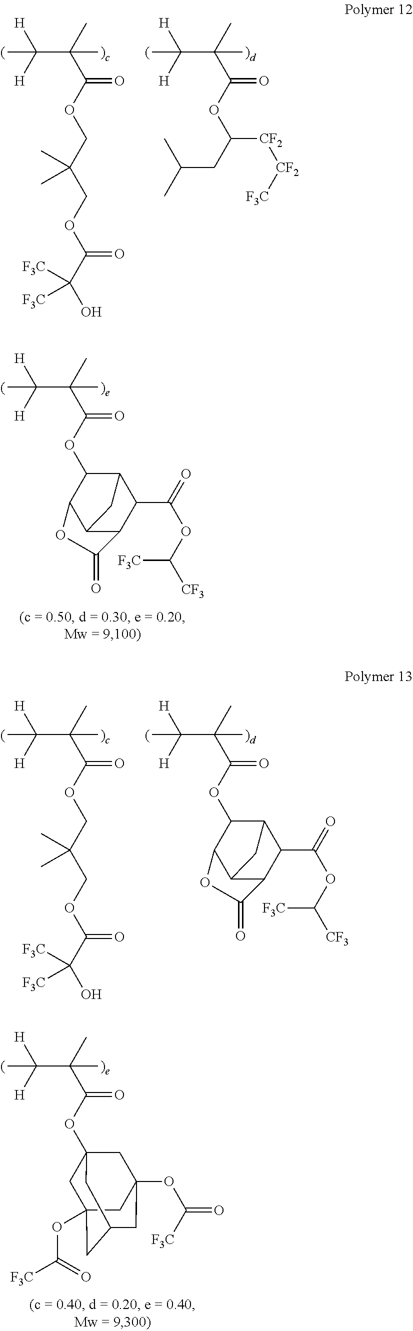

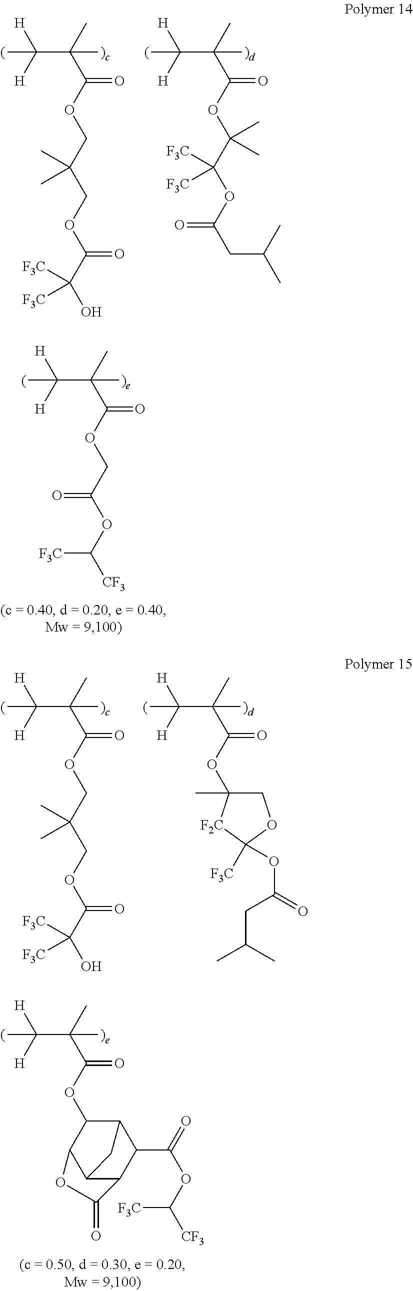

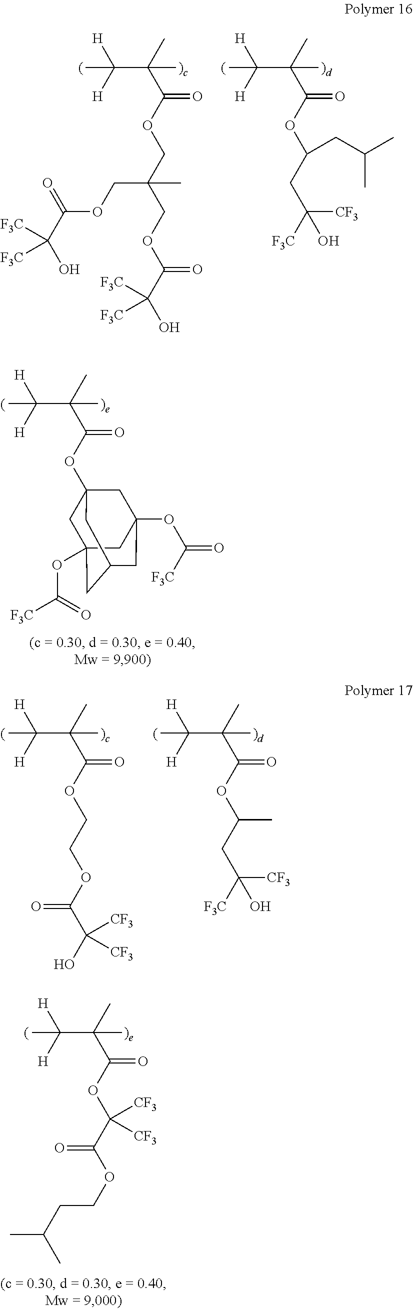

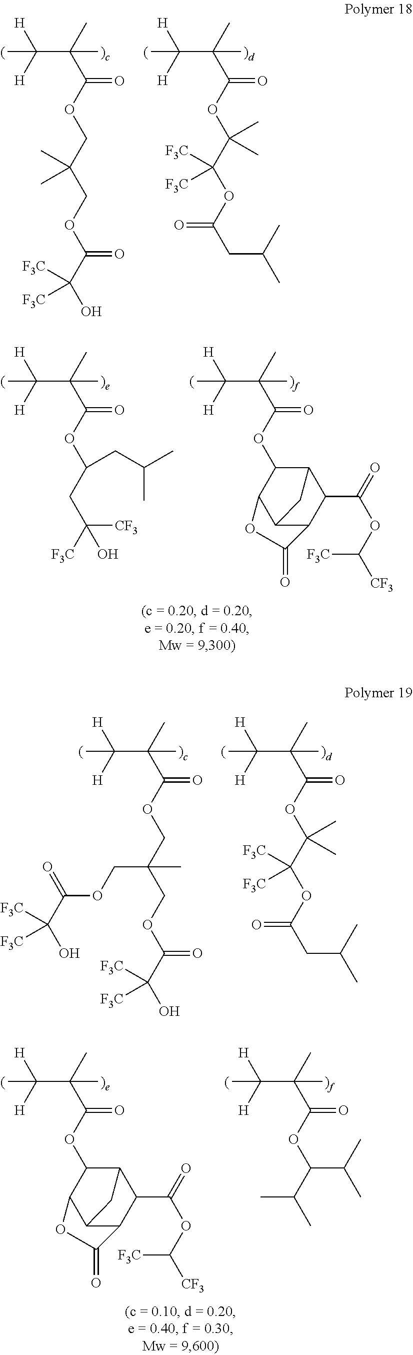

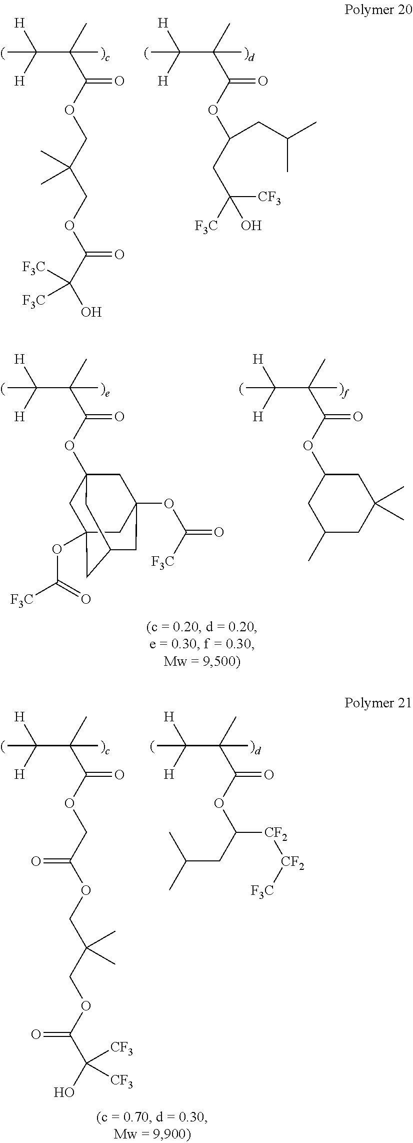

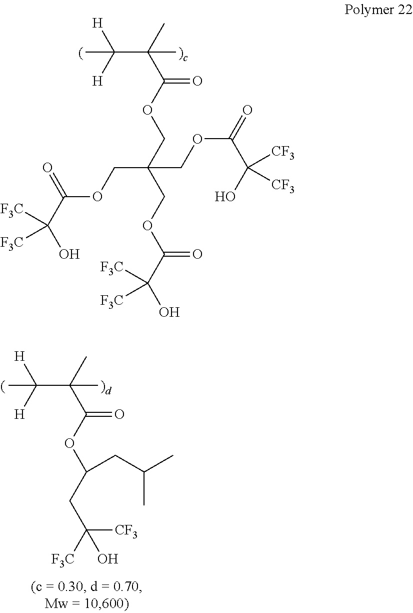

### Synthesis Example 3

### Synthesis Example 3-1

### Synthesis of sodium 3,3,3-trifluoro-2-hydroxy-2-trifluoro-methylpropionate

To 249 g (1.1 mol) of methyl 3,3,3-trifluoro-2-hydroxy-2-trifluoromethylpropionate were added 40 g (1 mol) of sodium hydroxide and 400 g of water. The mixture was heated and stirred at 70° C. for 12 hours. Water was removed in vacuum, and with toluene added, a minor amount of water was azeotroped off. The resulting crude crystals were dispersed in diisopropyl ether, filtered and dried, obtaining 213 g of crude crystals. This was ready for use in the subsequent step of reaction.

IR: 1661, 1406, 1351, 1270, 1211, 1169, 984, 805, 742, 675 cm⁻¹

¹H-NMR (600 MHz in DMSO-d₆): δ=6.3 (1H, s) ppm

¹⁹F-NMR (565 MHz in DMSO-d₆): δ=−73.7 (6F, s) ppm

### Synthesis Example 3-2

### Synthesis of 3,3,3-trifluoro-2-hydroxy-2-trifluoromethyl-propionic acid

Conc. sulfuric acid, 50 g, was added to 23.4 g of the crude crystals of sodium 3,3,3-trifluoro-2-hydroxy-2-trifluoromethylpropionate, followed by stirring. On distillation under a weakly applied pressure of nitrogen, 13 g of the desired carboxylic acid was obtained. Boiling point range: 160-165° C. The distillate solidified.

IR: 3444, 1754, 1227, 1158, 982, 724 cm⁻¹

¹⁹F-NMR (470 MHz in DMSO-d₆): δ=−75.0 (6F, s) ppm

### Synthesis Example 3-3

### Synthesis of tetramethylammonium 3,3,3-trifluoro-2-hydroxy-2-trifluoromethylpropanoate

To 12.4 g (0.055 mol) of methyl 3,3,3-trifluoro-2-hydroxy-2-trifluoromethylpropionate were added 18.5 g (0.053 mol) of 25 wt % tetramethylammonium hydroxide aqueous solution and 50 g of water. The mixture was heated and stirred at 55° C. for 4 hours. Water was removed in vacuum, and with toluene added, a minor amount of water was azeotroped off. The resulting crude crystals were dispersed in diisopropyl ether, filtered and dried, obtaining 16 g of crude crystals.

IR: 1683, 1494, 1422, 1364, 1288, 1249, 1192, 1149, 978, 953, 790, 739, 675 cm⁻¹

¹H-NMR (600 MHz in DMSO-d₆): δ=3.1 (12H, s), 6.4 (1H, s) ppm

¹⁹F-NMR (565 MHz in DMSO-d₆): δ=−74.3 (6F, m) ppm

### Synthesis Example 3-4

### Synthesis of triphenylsulfonium 3,3,3-trifluoro-2-hydroxy-2-trifluoromethylpropanoate (PAG-1)

A mixture of 49.7 g (0.22 mol) of methyl 3,3,3-trifluoro-2-hydroxy-2-trifluoromethylpropionate, 8 g (0.2 mol) of sodium hydroxide, and 80 g of water was heated and stirred at 60° C. for 8 hours. The mixture was cooled to room temperature, whereupon an equivalent amount of hydrochloric acid was added for neutralization. An equivalent amount of triphenylsulfonium chloride aqueous solution, 80 g of methyl isobutyl ketone and 40 g of dichloromethane were added to the reaction mixture, from which the organic layer was separated. The organic layer was washed three times with 50 g of water. From the organic layer, the solvent was stripped off in vacuum. Diisopropyl ether was added to the residue for crystallization. On filtration and drying, 42 g of white crystals was obtained. Yield 44%. The target compound was analyzed by spectroscopy, with the data shown below.

IR: 3063, 1694, 1477, 1447, 1313, 1282, 1247, 1204, 1191, 1145, 1066, 996, 977, 787, 748, 738, 724, 682 cm⁻¹

¹H-NMR (500 MHz in DMSO-d₆): δ=6.4 (1H, s), 7.7-7.9 (15H, m) ppm

¹⁹F-NMR (470 MHz in DMSO-d₆): δ=−74.3 (6F, s) ppm

### Synthesis Example 3-5

### Synthesis of 4-tert-butylphenyldiphenylsulfonium 3,3,3-trifluoro-2-hydroxy-2-trifluoromethylpropanoate (PAG-2)

The procedure of Synthesis Example 3-4 was repeated except that 7 g (0.03 mol) of sodium 3,3,3-trifluoro-2-hydroxy-2-trifluoromethylpropionate crude crystal and an amount (corresponding to 0.02 mol) of 4-tert-butylphenyldiphenyl-sulfonium bromide aqueous solution were used. In this way, 9.4 g of white crystals was obtained. Yield 89%. The target compound was analyzed by spectroscopy, with the data shown below.

IR: 2965, 1687, 1589, 1491, 1478, 1445, 1401, 1312, 1286, 1245, 1206, 1188, 1146, 1070, 977, 966, 827, 787, 752, 738, 686 cm⁻¹

¹H-NMR (500 MHz in DMSO-d₆: δ=1.3 (9H, s), 6.4 (1H, s), 7.7-7.9 (14H, m) ppm

¹⁹F-NMR (470 MHz in DMSO-d₆): δ=−74.2 (6F, s) ppm

### Synthesis Example 3-6

### Synthesis of 4-tert-butoxyphenyldiphenylsulfonium 3,3,3-trifluoro-2-hydroxy-2-trifluoromethylpropanoate (PAG-3)

The procedure of Synthesis Example 3-5 was repeated except that 4-tert-butoxyphenyldiphenylsulfonium p-toluene-sulfonate was used instead of the 4-tert-butylphenyldiphenyl-sulfonium bromide solution in Synthesis Example 3-5. In this way, 10 g of white crystals was obtained. Yield 92%. The target compound was analyzed by spectroscopy, with the data shown below.

IR: 2986, 1692, 1579, 1479, 1446, 1396, 1370, 1312, 1289, 1246, 1193, 1155, 1143, 1069, 996, 977, 892, 787, 748, 738, 688 cm⁻¹

¹H-NMR (500 MHz in DMSO-d₆): δ=1.4 (9H, s), 6.4 (1H, s), 7.4 (2H, d), 7.7-7.9 (12H, m) ppm

¹⁹F-NMR (470 MHz in DMSO-d₆): δ=−74.1 (6F, s) ppm

### Preparation of Resist

### Examples 1-1 to 1-6 and Comparative Examples 1-1 to 1-3

A resist solution was prepared by combining a PAG in Synthesis Example, Polymer A or B as base resin, additive and solvent in accordance with the formulation shown in Table 1, dissolving the components, and filtering through a Teflon® filter having a pore size of 0.2 μm. The solvent contained 0.01 wt % of surfactant A.

The solvent, quencher, PAG, and alkali soluble surfactant (SF) in Table 1 are identified below.

### P-A

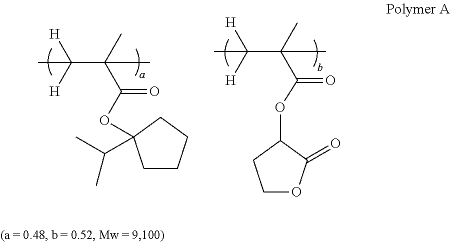

### P-B:

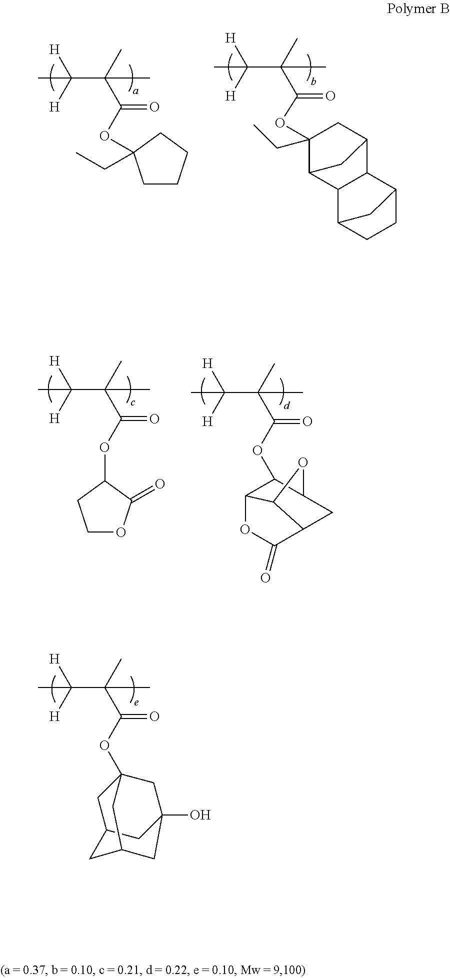 [0295] PAG-1, PAG-2, PAG-3: as synthesized above
PGMEA: propylene glycol monomethyl ether acetate
GBL: γ-butyrolactone
PAG-A: triphenylsulfonium 2-(adamantane-1-carbonyloxy)-1,1,3,3,3-pentafluoropropane-1-sulfonate
PAG-B: 4-tert-butylphenyldiphenylsulfonium 2-(adamantane-1-carbonyloxy)-1,1,3,3,3-pentafluoropropane-1-sulfonate
PAG-C: 4-tert-butylphenyldiphenylsulfonium 2-(4-oxo-adamantane-1-carbonyloxy)-1,1-difluoroethane-sulfonate
PAG-X: triphenylsulfonium nonafluoro-1-butansulfonate
PAG-Y: triphenylsulfonium 1-camphorsulfonate
PAG-Z: triphenylsulfonium heptafluoro-1-propanoate
BASE-1: 2,6-diisopropylaniline
SF-6, SF-8: Polymer 6, Polymer 8 synthesized above
SF-23: Polymer 23 (described in JP-A 2008-122932) poly(3,3,3-trifluoro-2-hydroxy-1,1-dimethyl-2-trifluoromethylpropyl methacrylate/1,1,1-trifluoro-2-hydroxy-6-methyl-2-trifluoro-methylhept-4-ylmethacrylate)
   Mw=7,300
   Mw/Mn=1.86

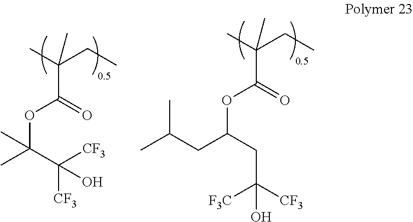

Surfactant A: 3-methyl-3-(2,2,2-trifluoroethoxymethyl)oxetane/tetrahydrofuran/2,2-dimethyl-1,3-propane diol copolymer (Omnova Solutions, Inc.) of the structural formula shown below.

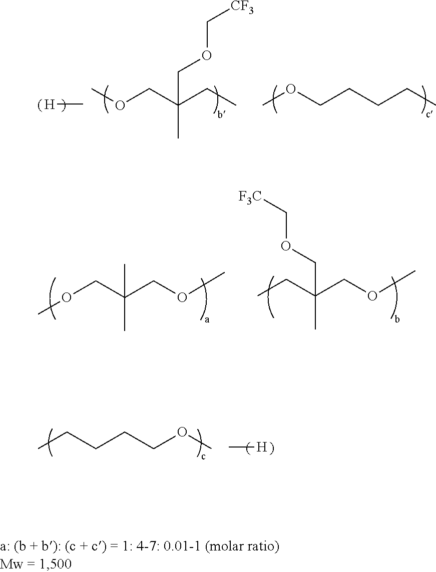

TABLE 1

| | Resist | Resin | PAG | PAG | Additive | Solvent 1 | Solvent 2 |
|---|---|---|---|---|---|---|---|
| | material | (pbw) | (pbw) | (pbw) | (pbw) | (pbw) | (pbw) |
| Example 1-1 | R-01 | P-A | PAG-A | PAG-1 | SF-23 | PGMEA | GBL |
| | | (80) | (6.34) | (3.68) | (5.0) | (1,728) | (192) |
| Example 1-2 | R-02 | P-A | PAG-B | PAG-1 | SF-23 | PGMEA | GBL |
| | | (80) | (6.88) | (3.68) | (5.0) | (1,728) | (192) |
| | | | | | BASE-1 | | |
| | | | | | (0.15) | | |
| Example 1-3 | R-03 | P-A | PAG-B | PAG-2 | SF-6 | PGMEA | GBL |
| | | (80) | (6.88) | (4.12) | (5.0) | (1,728) | (192) |
| Example 1-4 | R-04 | P-B | PAG-B | PAG-2 | SF-23 | PGMEA | GBL |
| | | (80) | (6.88) | (4.12) | (5.0) | (1,728) | (192) |
| Example 1-5 | R-05 | P-B | PAG-C | PAG-3 | SF-6 | PGMEA | GBL |
| | | (80) | (6.36) | (4.24) | (5.0) | (1,728) | (192) |
| Example 1-6 | R-06 | P-B | PAG-B | PAG-2 | SF-8 | PGMEA | GBL |
| | | (80) | (6.88) | (4.12) | (5.0) | (1,728) | (192) |
| Comparative | R-101 | P-A | PAG-A | PAG-Y | SF-23 | PGMEA | GBL |
| Example 1-1 | | (80) | (6.34) | (3.84) | (5.0) | (1,728) | (192) |
| Comparative | R-102 | P-B | PAG-A | PAG-Z | SF-23 | PGMEA | GBL |
| Example 1-2 | | (80) | (6.34) | (3.70) | (5.0) | (1,728) | (192) |
| Comparative | R-103 | P-A | PAG-X | PAG-1 | SF-23 | PGMEA | GBL |
| Example 1-3 | | (80) | (5.45) | (3.68) | (5.0) | (1,728) | (192) |

### Resist Evaluation

### Examples 2-1 to 2-6 and Comparative Examples 2-1 to 2-3

An antireflective coating solution (ARC-29A by Nissan Chemical Industries, Ltd.) was coated onto a silicon substrate and baked at 200° C. for 60 seconds to form an ARC film of 100 nm thick. The resist solution was spin coated onto the ARC and baked on a hot plate at 100° C. for 60 seconds to form a resist film of 90 nm thick. The resist film was exposed according to the ArF immersion lithography using an ArF excimer laser scanner (model NSR-S610C, Nikon Corp., NA 1.30, dipole illumination, 6% halftone phase shift mask). The resist film was baked (PEB) at an arbitrary temperature for 60 seconds and developed in a 2.38 wt % tetramethylammonium hydroxide aqueous solution for 60 seconds.

### Evaluation Method

The resist was evaluated by observing a 40-nm 1:1 line-and-space pattern under an electron microscope. The optimum dose (Eop) was a dose (mJ/cm²) which provided a line width of 40 nm. The profile of a pattern at the optimum dose was compared and judged passed or rejected according to the following criterion.
Passed: pattern of rectangular profile with perpendicular sidewall
Rejected: pattern of tapered profile with sharply graded sidewall or of top-rounded profile due to top loss

The width of lines of a 40-nm 1:1 line-and-space pattern was measured under SEM to determine a line width variation (30 points measured, 3σ value computed), which was reported as line width roughness (LWR). A smaller value of LWR indicates a line pattern with a less fluctuation and of better profile. In this test, the sample was rated good when LWR is equal to or less than 3.0 nm and poor when LWR is equal to or more than 3.1 nm.

The collapse limit was a minimum width (nm) of lines which could be resolved without collapse when the line width was reduced by increasing the exposure dose. A smaller value indicates better collapse resistance. In this test, the sample was rated good when the collapse limit is equal to or less than 33 nm and poor when the collapse limit is equal to or more than 34 nm.

Defects in the pattern as developed were inspected by a flaw detector KLA2800 (KLA-Tencor). A defect density (count/cm²) was computed by dividing the total number of detected defects by a detection area. The pattern formed was an iterated 40-nm 1:1 line-and-space pattern. The defect inspection conditions included light source UV, inspected pixel size 0.28 μm, and cell-to-cell mode. In this test, the sample was rated good for a defect density of less than 0.05 defect/cm² and poor for a density of equal to or more than 0.05 defect/cm².

The test results of the resist materials are shown in Table 2 together with the PEB temperature.

TABLE 2

| | | PEB temp. | Eop | | LER | Collapse | Defect density |
|---|---|---|---|---|---|---|---|
| | Resist | (° C.) | (mJ/cm²) | Profile | (nm) | limit (nm) | (count/cm²) |
| Example 2-1 | R-01 | 80 | 32 | Passed | Good 2.8 | Good 33 | Good 0.02 |
| Example 2-2 | R-02 | 80 | 38 | Passed | Good 3.0 | Good 30 | Good 0.03 |
| Example 2-3 | R-03 | 80 | 38 | Passed | Good 3.0 | Good 32 | Good 0.03 |
| Example 2-4 | R-04 | 95 | 40 | Passed | Good 2.8 | Good 33 | Good 0.04 |
| Example 2-5 | R-05 | 95 | 45 | Passed | Good 2.8 | Good 33 | Good 0.03 |
| Example 2-6 | R-06 | 95 | 40 | Passed | Good 3.0 | Good 32 | Good 0.03 |
| Comparative | R-101 | 80 | 26 | Passed | Poor 3.3 | Poor 36 | Poor 0.20 |
| Example 2-1 | | | | | | | |
| Comparative | R-102 | 95 | 30 | Passed | Poor 3.3 | Poor 34 | Poor 0.08 |
| Example 2-2 | | | | | | | |
| Comparative | R-103 | 80 | 28 | Rejected | Poor 3.7 | Good 31 | Good 0.03 |
| Example 2-3 | | | | | | | |

It is evident from the data of Table 2 that the resist compositions comprising specific sulfonium salts form patterns of good profile having a minimal LER, collapse resistance, and a low defect density.

### Measurement of Leach-Out from Resist Film

### Examples 3-1, 3-2, 4-1, 4-2 and Comparative Examples 3-1, 3-2, 4-1, 4-2

An amount of component leached out of resist film in immersion water was determined. Resist compositions (R-07 to 09) and comparative resist composition (R-104) were prepared as in Example 1, but in accordance with the formulation of Table 3. Each resist composition was spin coated onto a silicon substrate, baked at 100° C. for 60 seconds to form a photoresist film of 100 nm thick. In the test, the unexposed resist film was immersed in water because after exposure, no cations were detectable as a result of photo-reaction of PAG upon exposure.

TABLE 3

| | | | | | Sol- | Sol- |
|---|---|---|---|---|---|---|
| | Resist | Resin | PAG | Additive | vent 1 | vent 2 |
| | material | (pbw) | (pbw) | (pbw) | (pbw) | (pbw) |
| Example 3-1 | R-07 | P-A | PAG-1 | SF-23 | PGMEA | GBL |
| | | (80) | (4.60) | (5.0) | (1,944) | (216) |
| Example 3-2 | R-08 | P-A | PAG-2 | SF-6 | PGMEA | GBL |
| | | (80) | (5.14) | (5.0) | (1,944) | (216) |
| Comparative | R-09 | P-A | PAG-Y | SF-6 | PGMEA | GBL |
| Example 3-1 | | (80) | (4.80) | (5.0) | (1,944) | (216) |
| Comparative | R-104 | P-A | PAG-Z | SF-6 | PGMEA | GBL |
| Example 3-2 | | (80) | (4.62) | (5.0) | (1,944) | (216) |

Using WEXA-2 system (IMEC), the leaching solution was recovered from the resist film. Specifically, the resist film was chucked by vacuum suction to a stage provided with five slits of 5 mm deep and 50 mm long. Using a syringe pump (Harvard Apparatus), the leaching solution was recovered at a different flow volume and flow rate for each slit as shown in Table 4. The concentration of PAG cation in the leaching solution was quantitatively determined by LC-MS analyzer (Agilent Technologies).

TABLE 4

| | Flow volume | Flow rate |
|---|---|---|
| Slit | (ml) | (ml/min) |
| 1 | 2.65 | 35 |
| 2 | 3.0 | 25 |
| 3 | 3.1 | 20 |
| 4 | 3.0 | 13 |
| 5 | 2.65 | 4 |

From the cation concentration measured for each slit and the immersion time, a relationship of the leach-out amount to the immersion time was approximated to the following equation:

Leach-out amount Y=A×B×exp(−Bt)

wherein A is a saturation leach-out amount (mol/cm²), B is a time constant (s⁻¹), and t is an immersion time (s), from which constants A and B were determined.

Table 5 shows the measurement results of initial cation dissolution rate: A×B (mol/cm²·s) at t=0. With respect to the equation and computation method, reference should be made to Proc. SPIE, 6154, 186 (2006).

The sample was rated “Rejected” for a cation dissolution rate in excess of 1.6×10⁻¹² (mol/cm²·s) and “Passed” for a lower rate. The results are also shown in Table 5.

TABLE 5

| | Resist | Cation dissolution | |
|---|---|---|---|
| | material | rate (mol/cm²·s) | Rating |
| Example 4-1 | R-07 | 9.7 × 10⁻¹³ | Passed |
| Example 4-2 | R-08 | 7.5 × 10⁻¹³ | Passed |
| Comparative Example 4-1 | R-09 | 3.2 × 10⁻¹² | Rejected |
| Comparative Example 4-2 | R-104 | 2.7 × 10⁻¹² | Rejected |

It is seen from the data of Table 5 that the resist compositions within the scope of the invention are effective for preventing cations from being leached out during the immersion lithography using water. For the immersion lithography, little changes of the pattern profile and few damages to the exposure tool are expectable.

Japanese Patent Application No. 2011-171551 is incorporated herein by reference.

Although some preferred embodiments have been described, many modifications and variations may be made thereto in light of the above teachings. It is therefore to be understood that the invention may be practiced otherwise than as specifically described without departing from the scope of the appended claims.

1. A chemically amplified positive resist composition for ArF immersion lithography, comprising
(A) a sulfonium salt of 3,3,3-trifluoro-2-hydroxy-2-trifluoromethylpropionic acid having the general formula (1-1): 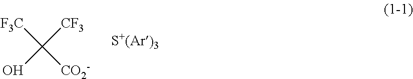
wherein Ar′ is a substituted or unsubstituted C₆-C₂₀ aryl group which may contain a heteroatom, or a plurality of Ar′ groups may bond directly or via an oxygen atom, methylene, sulfone or carbonyl moiety to form an aromatic-containing ring with the sulfur atom to which they are attached,
(B) one or more acid generator having the general formula (1-2): 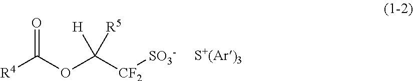
wherein R⁴ is a C₁-C₃₀ alkyl, alkenyl or aralkyl group which may contain a heteroatom, R⁵ is hydrogen or trifluoromethyl, and Ar′ is as defined above,
(C) a base resin having an acidic functional group protected with an acid labile group, which is insoluble or substantially insoluble in alkaline developer, but turns soluble in alkaline developer upon deprotection of the acid labile group, and
(D) an organic solvent.

2. A chemically amplified positive resist composition for ArF immersion lithography, comprising
(A) a sulfonium salt of 3,3,3-trifluoro-2-hydroxy-2-trifluoromethylpropionic acid having the general formula (1-1): 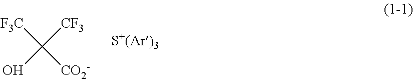
wherein Ar′ is a substituted or unsubstituted C₆-C₂₀ aryl group which may contain a heteroatom, or a plurality of Ar′ groups may bond directly or via an oxygen atom, methylene, sulfone or carbonyl moiety to form an aromatic-containing ring with the sulfur atom to which they are attached,
(C′) a base resin having an acidic functional group protected with an acid labile group, which is insoluble or substantially insoluble in alkaline developer, but turns soluble in alkaline developer upon deprotection of the acid labile group, the base resin comprising recurring units having the general formula (1-2′): 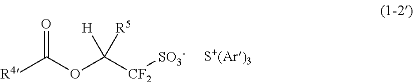
wherein R^{4′} is a backbone portion constituting some recurring units of the base resin, R⁵ is hydrogen or trifluoromethyl, and Ar′ is as defined above, and
(D) an organic solvent.

3. The resist composition of claim 1, further comprising as a surfactant a polymer comprising recurring units having the general formula (1a): 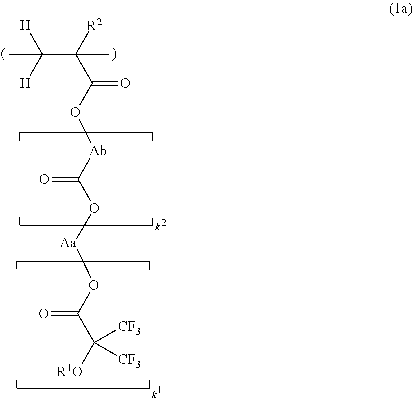
wherein R¹ is hydrogen or a straight, branched or cyclic C₁-C₂₀ monovalent hydrocarbon group in which a constituent moiety —CH₂— may be replaced by —O— or —C(═O)—, R² is hydrogen, fluorine, methyl or trifluoromethyl, Aa is a straight, branched or cyclic C₁-C₂₀ hydrocarbon or fluorinated hydrocarbon group having a valence of k¹+1, Ab is a straight, branched or cyclic C₁-C₆ divalent hydrocarbon group, k¹ is an integer of 1 to 3, and k² is 0 or 1.

4. The resist composition of claim 1 wherein the base resin comprises recurring units having an acid labile group represented by the general formula (3) and recurring units of at least one type selected from the general formulae (4) to (6): 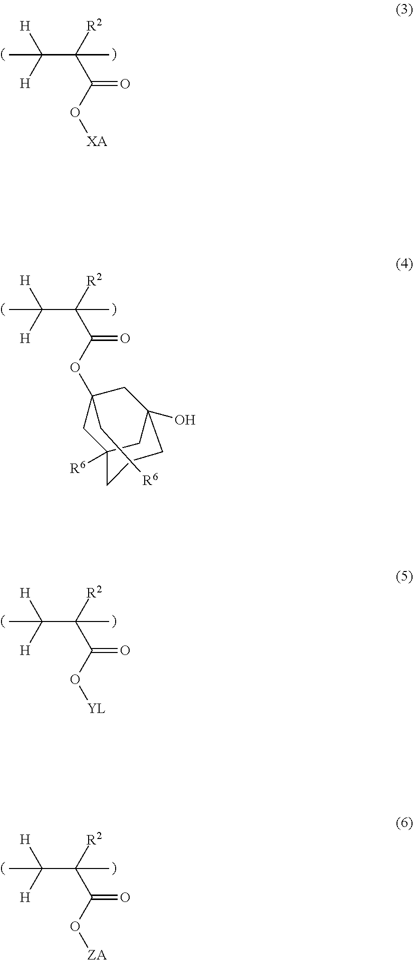
wherein R² is hydrogen, fluorine, methyl or trifluoromethyl, XA is an acid labile group, R⁶ is each independently hydrogen or hydroxyl, YL is a substituent group having a lactone structure, ZA is hydrogen, a C₁-C₁₅ fluoroalkyl group or C₁-C₁₅ fluoroalcohol-containing substituent group.

5. A pattern forming process comprising the steps of applying the resist composition of claim 1 onto a substrate, prebaking to form a resist film, exposing the resist film to high-energy radiation through a photomask while interposing water between the substrate and a projection lens, optionally baking, and developing in a developer.

6. A method for synthesizing a sulfonium salt of 3,3,3-trifluoro-2-hydroxy-2-trifluoromethylpropionic acid having the general formula (1-1): 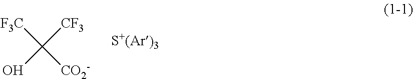
wherein Ar′ is a substituted or unsubstituted C₆-C₂₀ aryl group which may contain a heteroatom, or a plurality of Ar′ groups may bond directly or via an oxygen atom, methylene, sulfone or carbonyl moiety to form an aromatic-containing ring with the sulfur atom to which they are attached,
said method comprising the steps of providing methyl 3,3,3-trifluoro-2-hydroxy-2-trifluoromethylpropionate, effecting hydrolysis reaction into 3,3,3-trifluoro-2-hydroxy-2-trifluoromethylpropionic acid or a salt thereof, and obtaining the desired sulfonium salt therefrom.

7. A resist composition comprising a sulfonium salt of 3,3,3-trifluoro-2-hydroxy-2-trifluoromethylpropionic acid having the general formula (1-1) as synthesized by the method of claim 6.
